SEQUENCE LISTING (55 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 112 aa, Artificial Sequence
  Synthetic Construct
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 4 - protein, 103 aa, Artificial Sequence
  Synthetic Construct
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 5 - protein, 6 aa, Artificial Sequence
  Synthetic Construct
GYAFSY

SEQ ID NO: 6 - protein, 8 aa, Artificial Sequence
  Synthetic Construct
FPGDGDTD

SEQ ID NO: 7 - protein, 10 aa, Artificial Sequence
  Synthetic Construct
NVFDGYWLVY

SEQ ID NO: 8 - protein, 16 aa, Artificial Sequence
  Synthetic Construct
RSSKSLLHSNGITYLY

SEQ ID NO: 9 - protein, 7 aa, Artificial Sequence
  Synthetic Construct
QMSNLVS

SEQ ID NO: 10 - protein, 9 aa, Artificial Sequence
  Synthetic Construct
AQNLELPYT

SEQ ID NO: 11 - protein, 119 aa, Artificial Sequence
  Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 12 - protein, 115 aa, Artificial Sequence
  Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 13 - protein, 448 aa, Artificial Sequence
  Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 219 aa, Artificial Sequence
  Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, Artificial Sequence
  Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, Artificial Sequence
  Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 17 - protein, 448 aa, Artificial Sequence; identical to SEQ ID NO: 13
  Synthetic Construct

SEQ ID NO: 18 - protein, 219 aa, Artificial Sequence; identical to SEQ ID NO: 14
  Synthetic Construct

SEQ ID NO: 19 - protein, 117 aa, Artificial Sequence [sequence omitted]

SEQ ID NO: 20 - protein, 112 aa, Artificial Sequence
  Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 21 - protein, 10 aa, Artificial Sequence
  Synthetic Construct
GYTFSSYWIE

SEQ ID NO: 22 - protein, 18 aa, Artificial Sequence
  Synthetic Construct
GEILPGGGDTNYNEIFKG

SEQ ID NO: 23 - protein, 10 aa, Artificial Sequence
  Synthetic Construct
TRRVPIRLDY

SEQ ID NO: 24 - protein, 15 aa, Artificial Sequence
  Synthetic Construct
KASQSVDYEGDSFLN

SEQ ID NO: 25 - protein, 7 aa, Artificial Sequence
  Synthetic Construct
AASNLES

SEQ ID NO: 26 - protein, 9 aa, Artificial Sequence
  Synthetic Construct
QQSNEDPLT

SEQ ID NO: 27 - protein, 25 aa, Artificial Sequence [sequence omitted]

SEQ ID NO: 28 - protein, 13 aa, Artificial Sequence
  Synthetic Construct
WVRQAPGKGLEWI

SEQ ID NO: 29 - protein, 30 aa, Artificial Sequence
  Synthetic Construct
RATFSADTSKNTAYLQMNSLRAEDTAVYYC

SEQ ID NO: 30 - protein, 11 aa, Artificial Sequence
  Synthetic Construct
WGQGTLVTVSS

SEQ ID NO: 31 - protein, 23 aa, Artificial Sequence
  Synthetic Construct
DIQLTQSPSSLSASVGDRVTITC

SEQ ID NO: 32 - protein, 15 aa, Artificial Sequence
  Synthetic Construct
WYQQKPGKAPKLLIY

SEQ ID NO: 33 - protein, 32 aa, Artificial Sequence
  Synthetic Construct
GVPSRFSGSGSGTDFTLTISSLQPEDFATYYC

SEQ ID NO: 34 - protein, 11 aa, Artificial Sequence
  Synthetic Construct
FGQGTKVEIKR

SEQ ID NO: 35 - protein, 218 aa, Artificial Sequence
  Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, Artificial Sequence
  Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, Artificial Sequence
  Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, Artificial Sequence
  Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 39 - protein, 447 aa, Artificial Sequence
  Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 40 - protein, 119 aa, Artificial Sequence
  Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 41 - protein, 119 aa, Artificial Sequence
  Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 119 aa, Artificial Sequence; identical to SEQ ID NO: 11
  Synthetic Construct

SEQ ID NO: 43 - protein, 119 aa, Artificial Sequence
  Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 44 - protein, 119 aa, Artificial Sequence
  Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 45 - protein, 119 aa, Artificial Sequence
  Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 46 - protein, 119 aa, Artificial Sequence
  Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 47 - protein, 119 aa, Artificial Sequence
  Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 48 - protein, 119 aa, Artificial Sequence
  Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 49 - protein, 119 aa, Artificial Sequence
  Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 50 - protein, 119 aa, Artificial Sequence
  Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 51 - protein, 119 aa, Artificial Sequence
  Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 52 - protein, 119 aa, Artificial Sequence
  Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 53 - protein, 119 aa, Artificial Sequence
  Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 54 - protein, 119 aa, Artificial Sequence
  Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 115 aa, Artificial Sequence; identical to SEQ ID NO: 12
  Synthetic Construct

(19) United States
(12) Patent Application Publication
(10) Pub. No.: US 2021/0115141 A1
(43) Pub. Date: Apr. 22, 2021

(54) METHODS OF USING ANTI-CD79B IMMUNOCONJUGATES TO TREAT DIFFUSE LARGE B-CELL LYMPHOMA

(71) Applicants: Genentech, Inc., South San Francisco, CA (US); Hoffmann-La Roche Inc., Little Falls, NJ (US)

(72) Inventors: Juana Elva HERNANDEZ MONTALVO, Basel (CH); Naseer Ul Din QAYUM, London (GB); Jamie Harue HIRATA, South San Francisco, CA (US)

(73) Assignees: Genentech, Inc., South San Francisco, CA (US); Hoffmann-La Roche Inc., Little Falls, NJ (US)

(21) Appl. No.: 17/074,471

(22) Filed: Oct. 19, 2020

Related U.S. Application Data
(60) Provisional application No. 63/036,929, filed on Jun. 9, 2020.
(60) Provisional application No. 62/923,359, filed on Oct. 18, 2019.

(51) Int. Cl. C07K 16/28; A61P 35/00; A61K 31/7068; A61K 31/555; A61K 47/68; A61K 9/00

(52) U.S. Cl. CPC C07K 16/2803; A61P 35/00; C07K 16/2887; A61K 31/7068; A61K 2039/505; A61K 47/6803; A61K 47/6849; A61K 9/0019; A61K 31/555

(57) ABSTRACT

Provided herein are methods of treating B-cell proliferative disorders (such as diffuse large B-cell lymphoma (DLBCL)) using immunoconjugates comprising anti-CD79b antibodies in combination with an anti-CD20 antibody (such as rituximab) and one or more chemotherapeutic agents (such as gemcitabine and oxaliplatin).

### CROSS-REFERENCE TO RELATED APPLICATIONS

This application claims the benefit of U.S. Provisional Application No. 62/923,359, filed Oct. 18, 2019, and U.S. Provisional Application No. 63/036,929, filed Jun. 9, 2020, each of which is hereby incorporated by reference in its entirety.

### FIELD OF THE INVENTION

The present disclosure relates to methods of treating B-cell proliferative disorders, e.g., Diffuse Large B-Cell Lymphoma (DLBCL) by administering an immunoconjugate comprising an anti-CD79b antibody in combination with an anti-CD20 antibody (e.g., rituximab) and one or more chemotherapeutic agents (e.g., gemcitabine and oxaliplatin).

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392049100SEQLIST.TXT, date recorded: Oct. 16, 2020, size: 60 KB).

### BACKGROUND OF THE INVENTION

Non-Hodgkin lymphoma (NHL) is the most common hematologic malignancy in the world and the thirteenth most common cancer overall (Bray et al., (2018) CA Cancer J Clin, 68:394-424). Diffuse large B-cell lymphoma (DLBCL) is an aggressive subtype of NHL, accounting for approximately 32.5% of all NHL cases. DLBCL originates from mature B-cells and has a median survival of ≤1 year in untreated patients (Rovira et al., (2015) Ann Hematol, 378:1396-1407). A majority of DLBCL cells express CD20, a membrane antigen that is important in cell cycle initiation and differentiation (Anderson et al., (1984) Blood, 63:1424-1433).

First-line treatment of DLBCL has consisted of an anti-CD20 monoclonal antibody treatment in combination with a multi-agent chemotherapy (National Comprehensive Cancer Network 2018; Shen et al., (2018) Lancet vol 5, e264). For patients who are not cured by first-line therapy, high-dose chemotherapy followed by autologous stem cell transplantation offers a second chance for long-term remission. For relapsed/refractory (R/R) DLBCL patients who are not eligible for stem cell transplantation due to age, comorbidities, or other factors, there are different treatment options, including various chemoimmunotherapies. These chemoimmunotherapies, however, tend to be used with the goal of palliation rather than long-term survival. Recently approved treatments for the R/R DLBCL setting include CAR-T therapies and polatuzumab vedotin-piiq in combination with bendamustine and rituximab.

Approximately half of patients with relapsed DLBCL fail to respond to second-line therapy because of refractory disease (Gisselbrecht et al., (2010) J Clin Oncol, 28:4184-4190). Patients who either relapse after or are ineligible for stem cell transplantation because of refractory disease or frailty have poor outcomes. In addition, a significant number of relapsed/refractory patients are ineligible for aggressive therapy because of age, comorbidities, or other factors. While salvage therapies for relapsed or refractory DLBCL have shown encouraging results with respect to rates of response to therapy, long term survival of patients with relapsed or refractory DLBCL remains limited (Lopez et al., (2007) European J of Haematology 80:127-32; Gnaoui et al., (2007) Ann Oncol 18:1363-68; Mounier et al., (2013) Haematologica 98(11)1726-31). Thus, there is a need in the art for new therapeutic approaches in patients with relapsed or refractory DLBCL.

All references cited herein, including patent applications and publications, are hereby incorporated by reference in their entirety.

### SUMMARY

In one aspect, provided herein is a method for treating diffuse large B-cell lymphoma (DLBCL) in a human in need thereof comprising administering to the human an effective amount of:

(a) an immunoconjugate comprising the formula

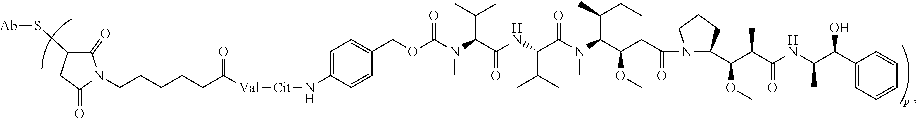

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin. In certain embodiments, p is between 2 and 5. In certain embodiments, p is 2.

In some embodiments, the immunoconjugate is administered at a dose that is from about 1 mg/kg to about 5 mg/kg. In some embodiments, the immunoconjugate is administered at a dose of about 1.2 mg/kg, about 1.8 mg/kg, about 2.4 mg/kg, about 3.6 mg/kg, or about 4.8 mg/kg. In some embodiments, the immunoconjugate is administered at a dose of about 1.8 mg/kg.

In some embodiments, which may be combined with any of the preceding embodiments, the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m². In certain embodiments, the immunoconjugate, the rituximab, the gemcitabine, and the oxaliplatin are administered for one or more 21-day cycles. In certain embodiments, the immunoconjugate is administered at a dose of about 1.8 mg/kg each cycle, the rituximab is administered at a dose of about 375 mg/m² each cycle, the gemcitabine is administered at a dose of about 1000 mg/m² each cycle, and the oxaliplatin is administered at a dose of about 100 mg/m² each cycle. In certain embodiments, the immunoconjugate and the rituximab are administered intravenously on Day 1 of each 21-day cycle, and the gemcitabine and the oxaliplatin are administered intravenously on Day 2 of each 21-day cycle. In certain embodiments, the rituximab is administered before the immunoconjugate. In certain embodiments, the gemcitabine is administered before the oxaliplatin. In certain embodiments, the immunoconjugate, the rituximab, the gemcitabine, and the oxaliplatin are administered for up to eight 21-day cycles. In certain embodiments, the immunoconjugate, the rituximab, the gemcitabine, and the oxaliplatin are administered for eight 21-day cycles.

In another aspect, provided herein is a method for treating diffuse large B-cell lymphoma (DLBCL) in a human in need thereof comprising administering to the human an effective amount of:

(a) an immunoconjugate comprising the formula

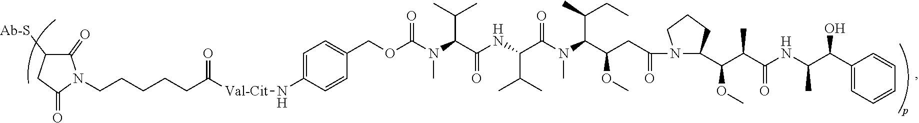

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

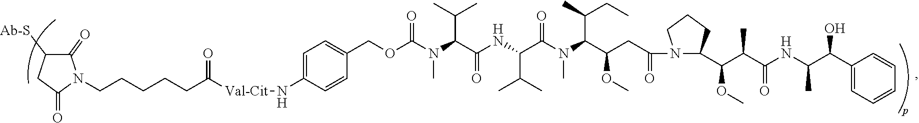

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

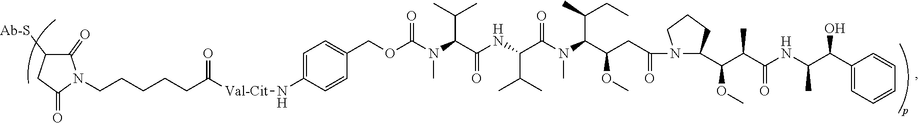

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.4 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

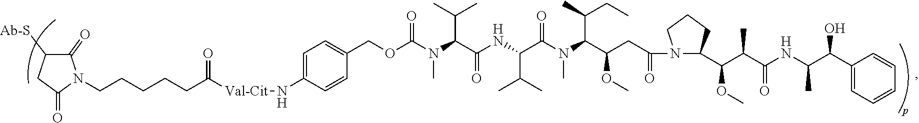

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.4 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

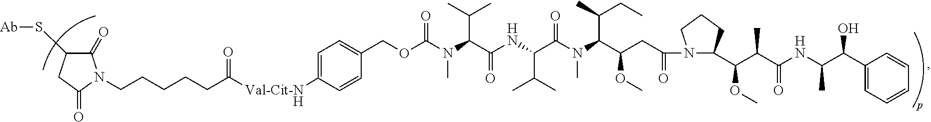

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 75 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

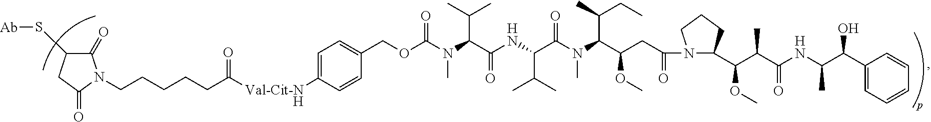

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 75 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

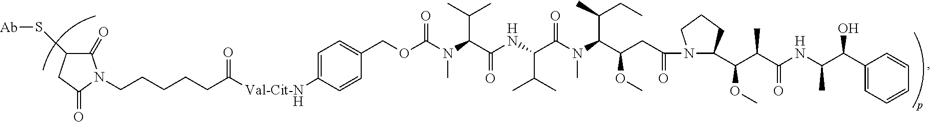

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.4 mg/kg, the rituximab is administered at a dose of about 375 mg/m², the gemcitabine is administered at a dose of about 1000 mg/m², and the oxaliplatin is administered at a dose of about 75 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is a method for treating diffuse large B-cell lymphoma (DLBCL) in a human in need thereof comprising administering to the human an effective amount of:

(a) an immunoconjugate comprising the formula

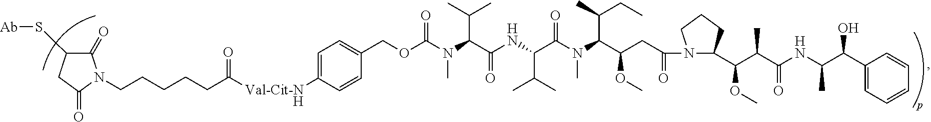

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.4 mg/kg, the rituximab is administered at a dose of about 375 mg/m², the gemcitabine is administered at a dose of about 1000 mg/m², and the oxaliplatin is administered at a dose of about 75 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In some embodiments, which may be combined with any of the preceding embodiments, the immunoconjugate, the rituximab, the gemcitabine, and the oxaliplatin are administered for at least two, at least three, at least four, at least five, at least six, or at least seven 21-day cycles. In some embodiments, the immunoconjugate, the rituximab, the gemcitabine, and the oxaliplatin are administered for eight 21-day cycles. In some embodiments, the rituximab is administered before the immunoconjugate. In some embodiments, the gemcitabine is administered before the oxaliplatin. In some embodiments, the immunoconjugate is polatuzumab vedotin-piiq.

In some embodiments, the human has received at least one prior therapy for DLBCL. In some embodiments, the human has received at least one prior systemic therapy for DLBCL. In some embodiments, the human has received at least two prior therapies for DLBCL. In certain embodiments, the DLBCL is histologically-confirmed DLBCL, not otherwise specified (NOS) or the human has a history of transformation of indolent disease to DLBCL. In certain embodiments, the DLBCL is relapsed or refractory DLBCL. In certain embodiments, the human has an Eastern Cooperative Oncology Group (ECOG) performance status of 0, 1, or 2. In certain embodiments, the human does not have a planned autologous or allogeneic stem cell transplantation (SCT). In certain embodiments, the human has not had prior therapy with a combination of gemcitabine and a platinum-based agent. In certain embodiments, the human does not have peripheral neuropathy of greater than Grade 1 according to National Cancer Institute Common Terminology Criteria for Adverse Events, Version 5.0. In certain embodiments, the human does not have primary or secondary central nervous system lymphoma. In some embodiments, the human is not a candidate for hematopoietic stem cell transplantation (HSCT) prior to administration of the immunoconjugate, the rituximab, the gemcitabine, and the oxaliplatin. In some embodiments, the human is not a candidate for autologous hematopoietic stem cell transplantation (HSCT) prior to administration of the immunoconjugate, the rituximab, the gemcitabine, and the oxaliplatin. In some embodiments, the human has received at least two prior therapies for DLBCL. In some embodiments, the human has not received a prior therapy with polatuzumab vedotin-piiq for DLBCL. In some embodiments, the human is an adult. In some embodiments, the human adult has relapsed or refractory diffuse large B-cell lymphoma, not otherwise specified.

In certain embodiments, after administration of the immunoconjugate, the rituximab, the gemcitabine, and the oxaliplatin, the human does not experience peripheral neuropathy of Grade 3 or greater that does not resolve to Grade 1 or lower within 14 days. In certain embodiments, among a plurality of humans treated, after administration of the immunoconjugate, the rituximab, the gemcitabine, and the oxaliplatin, 33% or fewer experience peripheral neuropathy of Grade 3 or greater that does not resolve to Grade 1 or lower within 14 days. In certain embodiments, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days. In some embodiments, administration of the immunoconjugate, the rituximab, the gemcitabine, and the oxaliplatin does not result in peripheral neuropathy of Grade 3 or greater in the human that does not resolve to Grade 1 or lower within 14 days. In some embodiments, administration of the immunoconjugate, the rituximab, the gemcitabine, and the oxaliplatin does not result in peripheral neuropathy of Grade 4 or greater in the human. In some embodiments, administration of the immunoconjugate, the rituximab, the gemcitabine, and the oxaliplatin does not result in neurotoxicity of Grade 4 or greater in the human. In some embodiments, after administration of the immunoconjugate, the rituximab, the gemcitabine, and the oxaliplatin, the human does not experience peripheral neuropathy of Grade 4 or greater. In some embodiments, after administration of the immunoconjugate, the rituximab, the gemcitabine, and the oxaliplatin, the human does not experience neurotoxicity of Grade 4 or greater. In some embodiments, administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days. In some embodiments, administration of the immunoconjugate, the rituximab, the gemcitabine, and the oxaliplatin to a plurality of humans results in less than about 40% of the humans in the plurality experiencing peripheral neuropathy of Grade 3 or greater that does not resolve to Grade 1 or lower within 14 days. In some embodiments, administration of the immunoconjugate, the rituximab, the gemcitabine, and the oxaliplatin to a plurality of humans results in less than about 8% of the humans in the plurality experiencing peripheral neuropathy of Grade 3 or greater. In some embodiments, administration of the immunoconjugate, the rituximab, the gemcitabine, and the oxaliplatin to a plurality of humans results in less than about 6% of the humans in the plurality experiencing peripheral neuropathy that results in discontinuation of treatment with the immunoconjugate, the rituximab, the gemcitabine, and the oxaliplatin. In certain embodiments, the immunoconjugate, the rituximab, the gemcitabine, and the oxaliplatin have been administered for at least four 21-day cycles.

In another aspect, provided herein is a method for treating diffuse large B-cell lymphoma (DLBCL) in a human in need thereof comprising administering to the human an effective amount of: (a) an immunoconjugate comprising the formula

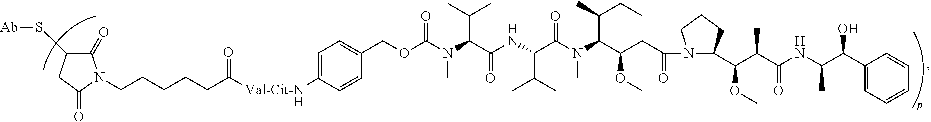

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein, after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

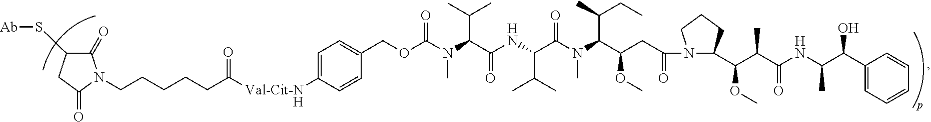

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein administration of the immunoconjugate, the rituximab, the gemcitabine, and the oxaliplatin does not result in peripheral neuropathy of Grade 3 or greater in the human that does not resolve to Grade 1 or lower within 14 days.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In another aspect, provided herein is a method for treating diffuse large B-cell lymphoma (DLBCL) in a human in need thereof comprising administering to the human an effective amount of: (a) an immunoconjugate comprising the formula

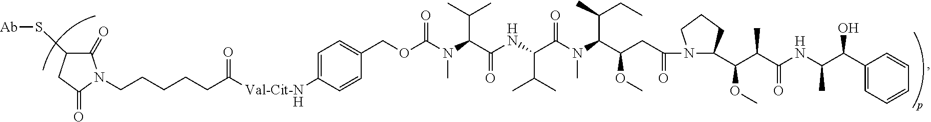

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

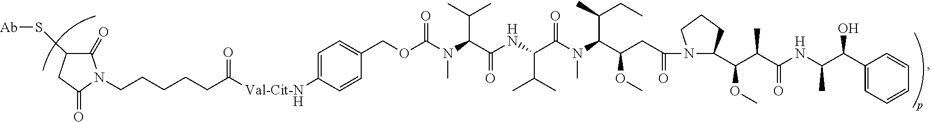

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein, administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In some embodiments, which may be combined with any of the preceding embodiments, the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m². In certain embodiments, the immunoconjugate, the rituximab, the gemcitabine, and the oxaliplatin are administered for one or more 21-day cycles. In certain embodiments, the immunoconjugate is administered at a dose of about 1.8 mg/kg each cycle, the rituximab is administered at a dose of about 375 mg/m² each cycle, the gemcitabine is administered at a dose of about 1000 mg/m² each cycle, and the oxaliplatin is administered at a dose of about 100 mg/m² each cycle. In certain embodiments, the immunoconjugate and the rituximab are administered intravenously on Day 1 of each 21-day cycle, and the gemcitabine and the oxaliplatin are administered intravenously on Day 2 of each 21-day cycle. In certain embodiments, the rituximab is administered before the immunoconjugate. In certain embodiments, the gemcitabine is administered before the oxaliplatin. In certain embodiments, the immunoconjugate, the rituximab, the gemcitabine, and the oxaliplatin are administered for up to eight 21-day cycles. In certain embodiments, the immunoconjugate, the rituximab, the gemcitabine, and the oxaliplatin are administered for eight 21-day cycles.

In another aspect, provided herein is a method for treating diffuse large B-cell lymphoma (DLBCL) in a human in need thereof comprising administering to the human an effective amount of: (a) an immunoconjugate comprising the formula

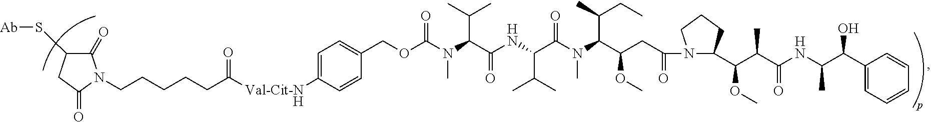

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

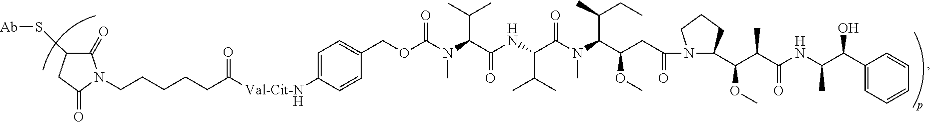

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

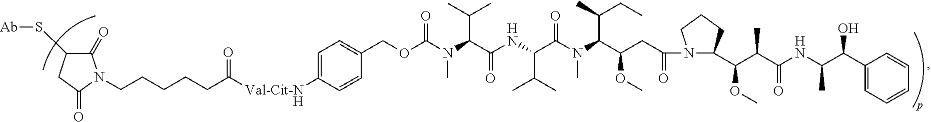

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

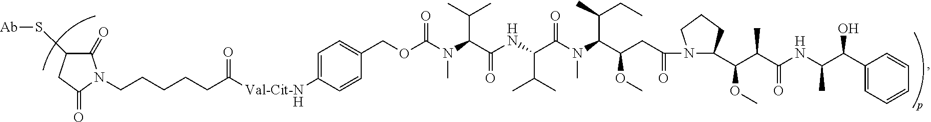

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

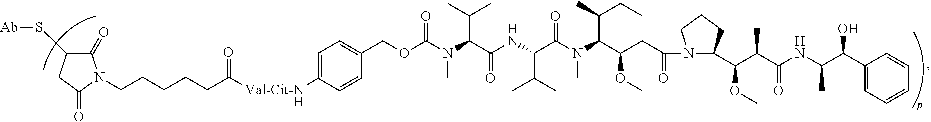

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

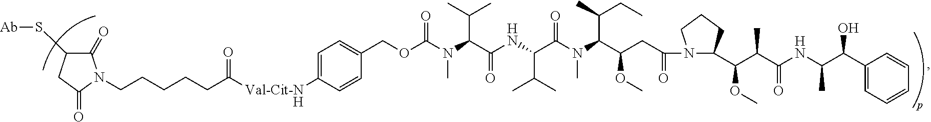

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

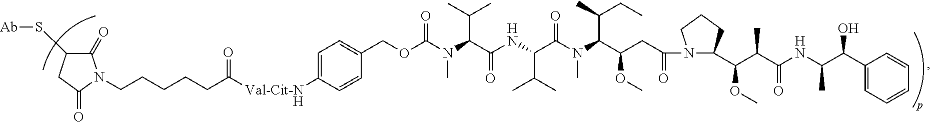

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

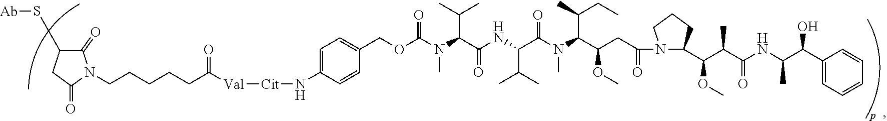

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In some embodiments, which may be combined with any of the preceding embodiments, the immunoconjugate, the rituximab, the gemcitabine, and the oxaliplatin have been administered for at least four 21-day cycles.

In some embodiments, which may be combined with any of the preceding embodiments, the immunoconjugate, the rituximab, the gemcitabine, and the oxaliplatin are administered for at least two, at least three, at least four, at least five, at least six, or at least seven 21-day cycles. In some embodiments, the immunoconjugate, the rituximab, the gemcitabine, and the oxaliplatin are administered for eight 21-day cycles. In some embodiments, the rituximab is administered before the immunoconjugate. In some embodiments, the gemcitabine is administered before the oxaliplatin.

In some embodiments, which may be combined with any of the preceding embodiments, the immunoconjugate is polatuzumab vedotin-piiq.

In some embodiments, which may be combined with any of the preceding embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin. In certain embodiments, p is between 2 and 5. In certain embodiments, p is 2.

In some embodiments, the human has received at least one prior therapy for DLBCL. In some embodiments, the human has received at least one prior systemic therapy for DLBCL. In some embodiments, the human has received at least two prior therapies for DLBCL. In certain embodiments, the DLBCL is histologically-confirmed DLBCL, not otherwise specified (NOS) or the human has a history of transformation of indolent disease to DLBCL. In certain embodiments, the DLBCL is relapsed or refractory DLBCL. In certain embodiments, the human has an Eastern Cooperative Oncology Group (ECOG) performance status of 0, 1, or 2. In certain embodiments, the human does not have a planned autologous or allogeneic stem cell transplantation (SCT). In certain embodiments, the human has not had prior therapy with a combination of gemcitabine and a platinum-based agent. In certain embodiments, the human does not have peripheral neuropathy of greater than Grade 1 according to National Cancer Institute Common Terminology Criteria for Adverse Events, Version 5.0. In certain embodiments, the human does not have primary or secondary central nervous system lymphoma. In some embodiments, the human is not a candidate for hematopoietic stem cell transplantation (HSCT) prior to administration of the immunoconjugate, the rituximab, the gemcitabine, and the oxaliplatin. In some embodiments, the human is not a candidate for autologous hematopoietic stem cell transplantation (HSCT) prior to administration of the immunoconjugate, the rituximab, the gemcitabine, and the oxaliplatin. In some embodiments, the human has received at least two prior therapies for DLBCL. In some embodiments, the human has not received a prior therapy with polatuzumab vedotin-piiq for DLBCL. In some embodiments, the human is an adult. In some embodiments, the human adult has relapsed or refractory diffuse large B-cell lymphoma, not otherwise specified.

In some embodiments, which may be combined with any of the preceding embodiments, administration of the immunoconjugate, the rituximab, the gemcitabine, and the oxaliplatin does not result in peripheral neuropathy of Grade 3 or greater in the human that does not resolve to Grade 1 or lower within 14 days. In some embodiments, administration of the immunoconjugate, the rituximab, the gemcitabine, and the oxaliplatin does not result in peripheral neuropathy of Grade 4 or greater in the human. In some embodiments, administration of the immunoconjugate, the rituximab, the gemcitabine, and the oxaliplatin does not result in neurotoxicity of Grade 4 or greater in the human. In some embodiments, after administration of the immunoconjugate, the rituximab, the gemcitabine, and the oxaliplatin, the human does not experience peripheral neuropathy of Grade 4 or greater. In some embodiments, after administration of the immunoconjugate, the rituximab, the gemcitabine, and the oxaliplatin, the human does not experience neurotoxicity of Grade 4 or greater. In some embodiments, administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days. In some embodiments, administration of the immunoconjugate, the rituximab, the gemcitabine, and the oxaliplatin to a plurality of humans results in less than about 40% of the humans in the plurality experiencing peripheral neuropathy of Grade 3 or greater that does not resolve to Grade 1 or lower within 14 days. In some embodiments, administration of the immunoconjugate, the rituximab, the gemcitabine, and the oxaliplatin to a plurality of humans results in less than about 8% of the humans in the plurality experiencing peripheral neuropathy of Grade 3 or greater. In some embodiments, administration of the immunoconjugate, the rituximab, the gemcitabine, and the oxaliplatin to a plurality of humans results in less than about 6% of the humans in the plurality experiencing peripheral neuropathy that results in discontinuation of treatment with the immunoconjugate, the rituximab, the gemcitabine, and the oxaliplatin. In some embodiments, the immunoconjugate, the rituximab, the gemcitabine, and the oxaliplatin have been administered for at least four 21-day cycles. In some embodiments, administration of the immunoconjugate, the rituximab, the gemcitabine, and the oxaliplatin results in a complete response in the human. In some embodiments, administration of the immunoconjugate, the rituximab, the gemcitabine, and the oxaliplatin results in a partial response in the human.

In some embodiments, which may be combined with any of the preceding embodiments, administration of the immunoconjugate, the rituximab, the gemcitabine, and the oxaliplatin results in progression-free survival of the human. In some embodiments, administration of the immunoconjugate, the rituximab, the gemcitabine, and the oxaliplatin results in progression-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 4 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 5 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 6 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 9.5 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 11 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 14 months after the start of treatment with the immunoconjugate, the rituximab, the gemcitabine, and the oxaliplatin. In some embodiments, administration of the immunoconjugate, the rituximab, the gemcitabine, and the oxaliplatin results in an increase in progression-free survival of the human compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in progression-free survival of the humans in the plurality compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin results in event-free survival of the human. In some embodiments, administration of the immunoconjugate, the rituximab, the gemcitabine, and the oxaliplatin results in event-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 4 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 5 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 6 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 9.5 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 11 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 14 months after the start of treatment with the immunoconjugate, the rituximab, the gemcitabine, and the oxaliplatin. In some embodiments, administration of the immunoconjugate, the rituximab, the gemcitabine, and the oxaliplatin results in an increase in event-free survival of the human compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in event-free survival of the humans in the plurality compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin results in survival of the human for at least about 10 months or more, at least about 11 months or more, at least about 12 months or more, at least about 13 months or more, at least about 14 months or more, or at least about 15 months or more after the start of treatment with the immunoconjugate, the rituximab, the gemcitabine, and the oxaliplatin. In some embodiments, administration of the immunoconjugate, the rituximab, the gemcitabine, and the oxaliplatin to a plurality of humans results in a median overall survival of the humans in the plurality of at least about 10 months or more, at least about 11 months or more, at least about 12 months or more, at least about 13 months or more, at least about 14 months or more, or at least about 15 months or more after the start of treatment with the immunoconjugate, the rituximab, the gemcitabine, and the oxaliplatin. In some embodiments, administration of the immunoconjugate, the rituximab, the gemcitabine, and the oxaliplatin results in an increase in survival of the human compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in the median overall survival of the humans in the plurality compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin does not result in tumor lysis syndrome in the human. In some embodiments, the methods provided herein further comprise administering a prophylactic treatment for tumor lysis syndrome to the human before, during, and/or after administration of the immunoconjugate, the rituximab, the gemcitabine, and the oxaliplatin to the human.

In some embodiments, which may be combined with any of the preceding embodiments, administration of the immunoconjugate, the rituximab, the gemcitabine, and the oxaliplatin does not result in an infection in the human. In some embodiments, the infection is a pneumocystis infection or a herpesvirus infection. In some embodiments, the methods provided herein further comprise administering a prophylactic treatment for infections to the human before, during, and/or after administration of the immunoconjugate, the rituximab, the gemcitabine, and the oxaliplatin to the human.

In some embodiments, which may be combined with any of the preceding embodiments, administration of the immunoconjugate, the rituximab, the gemcitabine, and the oxaliplatin does not result in neutropenia in the human. In some embodiments, the methods provided herein further comprise administering a prophylactic treatment for neutropenia to the human before, during, and/or after administration of the immunoconjugate, the rituximab, the gemcitabine, and the oxaliplatin to the human. In some embodiments, the prophylactic treatment for neutropenia comprises administering G-CSF to the human. In some embodiments, the methods provided herein further comprise stopping treatment with the immunoconjugate, the rituximab, the gemcitabine, and the oxaliplatin if Grade 3 or Grade 4 neutropenia occurs in the human after administration of the immunoconjugate, the rituximab, the gemcitabine, and the oxaliplatin. In some embodiments, the methods further comprise resuming treatment with the immunoconjugate, the rituximab, the gemcitabine, and the oxaliplatin if absolute neutrophil count (ANC) increases to >1000/μL in the human. In some embodiments, the methods further comprise administering one or more growth factors to treat neutropenia. In some embodiments, the one or more growth factors comprise G-CSF. In some embodiments, the methods further comprise resuming treatment with the immunoconjugate, the rituximab, the gemcitabine, and the oxaliplatin without a dose reduction of the immunoconjugate, the rituximab, the gemcitabine, and the oxaliplatin if ANC recovers to >1000/μL in the human on or before Day 7 of a 21-day cycle. In some embodiments, the methods further comprise resuming treatment with the immunoconjugate, the rituximab, the gemcitabine, and the oxaliplatin, wherein the dose of the immunoconjugate is reduced to 1.4 mg/kg if ANC recovers to >1000/μL in the human after Day 7 of a 21-day cycle. In some embodiments, the methods further comprise discontinuing treatment with the immunoconjugate, the rituximab, the gemcitabine, and the oxaliplatin if a prior dose reduction of the immunoconjugate, the rituximab, the gemcitabine, and the oxaliplatin occurred.

In some embodiments, which may be combined with any of the preceding embodiments, administration of the immunoconjugate, the rituximab, the gemcitabine, and the oxaliplatin does not result in Hepatitis B reactivation in the human. In some embodiments, the methods provided herein further comprise administering a prophylactic treatment for Hepatitis B reactivation to the human before, during, and/or after administration of the immunoconjugate, the rituximab, the gemcitabine, and the oxaliplatin to the human. In some embodiments, the methods provided herein further comprise administering anti-viral medication to the human if Hepatitis B reactivation is detected in the human.

In some embodiments, which may be combined with any of the preceding embodiments, administration of the immunoconjugate, the rituximab, the gemcitabine, and the oxaliplatin does not result in one or more adverse events selected from the group consisting of drug-induced liver injury, progressive multifocal leukoencephalopathy, systemic hypersensitivity reaction, anaphylactic reaction, anaphylactoid reaction, and second malignancy.

In some embodiments, which may be combined with any of the preceding embodiments, administration of the immunoconjugate, the rituximab, the gemcitabine, and the oxaliplatin to a plurality of humans results in an increase in the complete response rate (CRR) compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in an increase in the objective response rate (ORR) compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in an increase in the best overall response rate (BOR) compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in an increase in the duration of response (DOR) compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in a 1-year progression-free survival rate of at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 2-year progression-free survival rate of at least about 63% or greater, at least about 65% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 5-year progression-free survival rate of at least about 14% or greater, at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase in the 1-year, 2-year, 3.5-year, or 5-year progression-free survival rate compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in a 1-year overall survival rate of at least about 42% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 2-year overall survival rate of at least about 67% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 3.5-year overall survival rate of at least about 38% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 5-year overall survival rate of at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase in the 1-year, 2-year, 3.5-year, or 5-year overall survival rate compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in a 2-year event-free survival rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase in the 1-year, 2-year, 3.5-year, or 5-year event-free survival rate compared to a corresponding plurality of humans administered rituximab, gemcitabine, and oxaliplatin.

In some embodiments, which may be combined with any of the preceding embodiments, administration of the immunoconjugate, the rituximab, the gemcitabine, and the oxaliplatin to a plurality of humans results in an objective response rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality.

In some embodiments, which may be combined with any of the preceding embodiments, administration of the immunoconjugate, the rituximab, the gemcitabine, and the oxaliplatin to a plurality of humans results in a complete response rate of at least about 35% or greater, at least about 40% or greater, at least about 50% or greater, at least about 60% or greater, at least about 70% or greater, at least about 80% or greater, at least about 90% or greater, at least about 95% or greater, or about 100% of the humans in the plurality.

In some embodiments, which may be combined with any of the preceding embodiments, administration of the immunoconjugate, the rituximab, the gemcitabine, and the oxaliplatin to a plurality of humans results in a partial response rate of at least about 10% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, which may be combined with any of the preceding embodiments, administration of the immunoconjugate, the rituximab, the gemcitabine, and the oxaliplatin to a plurality of humans results in an increase in the partial response rate compared to a corresponding plurality of humans administered rituximab, gemcitabine, and oxaliplatin.

In another aspect, provided herein is a kit comprising an immunoconjugate comprising the formula

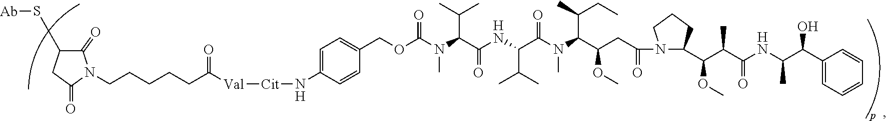

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with rituximab, gemcitabine, and oxaliplatin for treating a human in need thereof having diffuse large B-cell lymphoma (DLBCL) according to any of the preceding embodiments.

In another aspect, provided herein is a kit comprising polatuzumab vedotin-piiq for use in combination with rituximab, gemcitabine, and oxaliplatin for treating a human in need thereof having diffuse large B-cell lymphoma (DLBCL) according to according to any of the preceding embodiments.

In another aspect, provided herein is an immunoconjugate comprising the formula

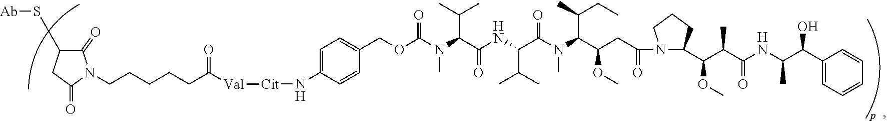

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

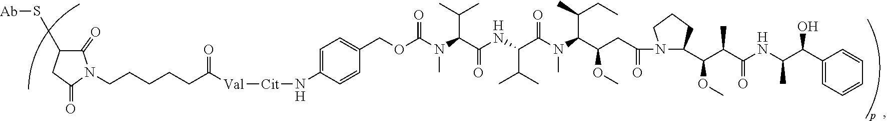

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

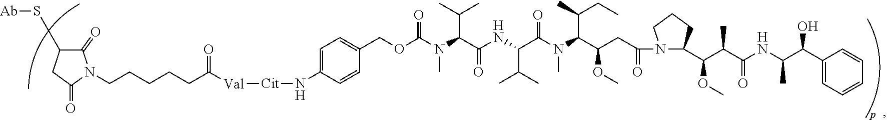

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

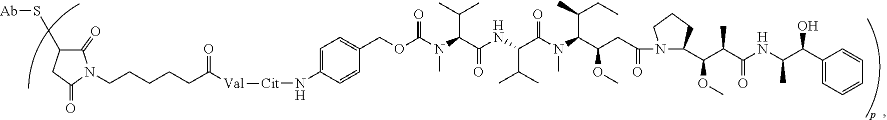

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is an immunoconjugate comprising the formula

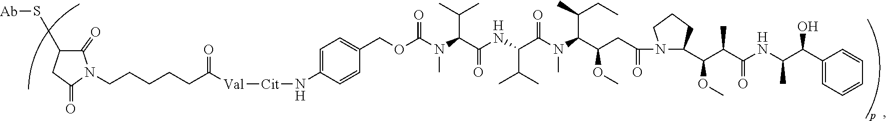

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is an immunoconjugate comprising the formula

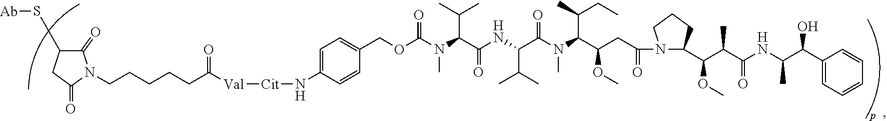

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is an immunoconjugate comprising the formula

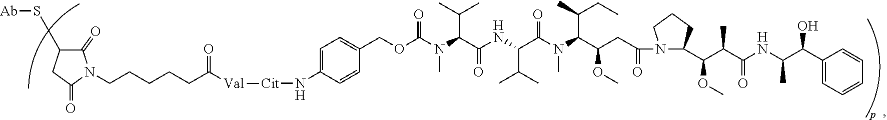

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is an immunoconjugate comprising the formula

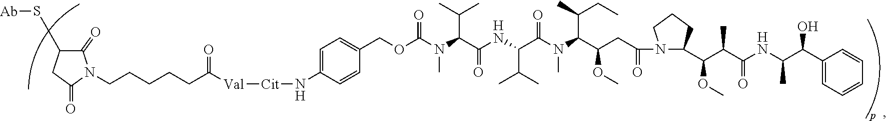

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

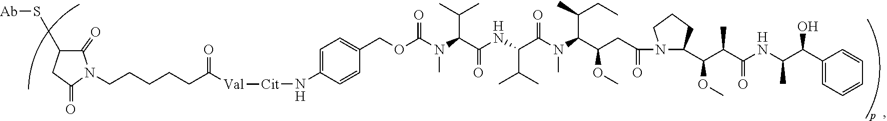

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

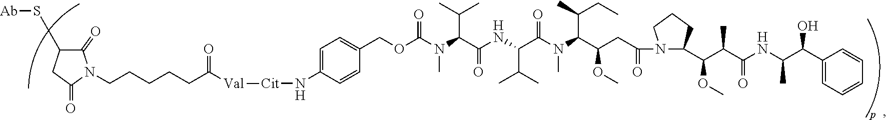

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

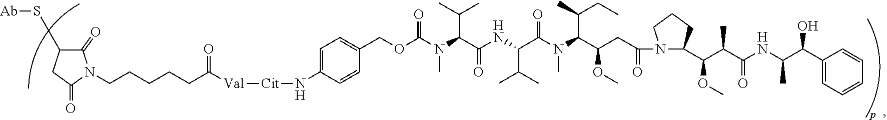

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

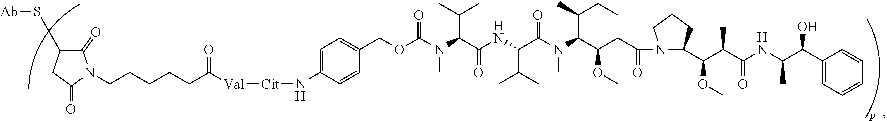

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

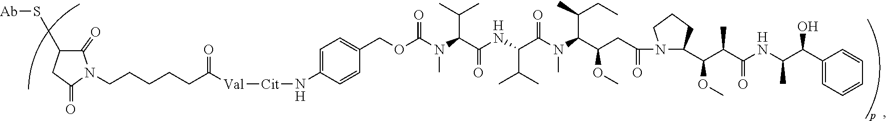

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

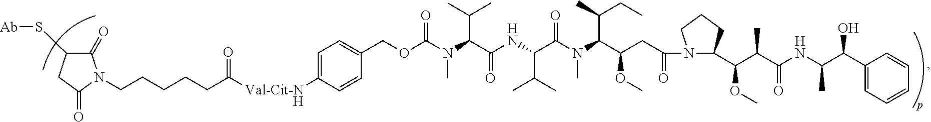

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

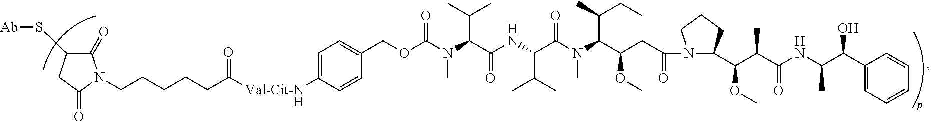

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

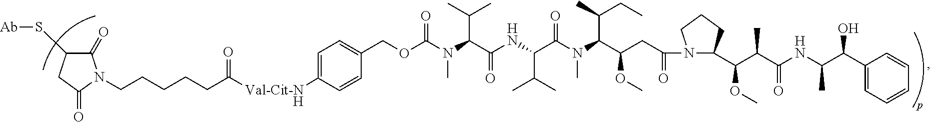

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

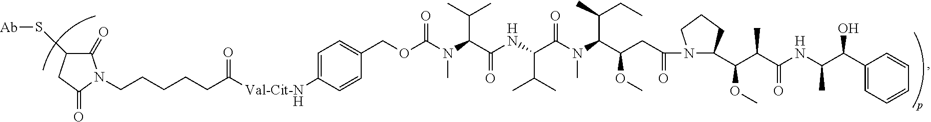

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, after administration of the immunoconjugate, the rituximab, the gemcitabine, and the oxaliplatin, the human does not experience peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

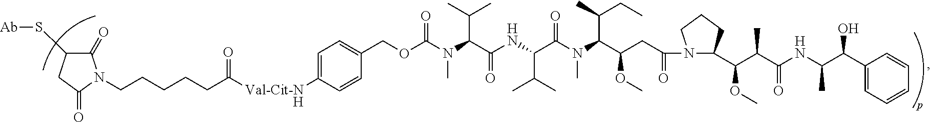

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

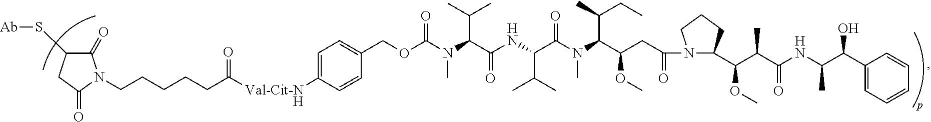

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

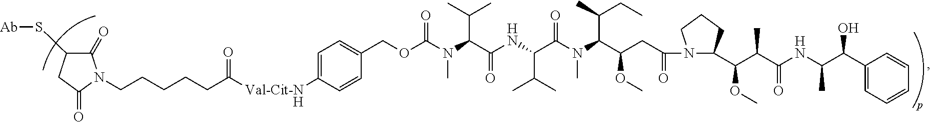

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In one aspect, provided herein is a method for treating diffuse large B-cell lymphoma (DLBCL) in a human in need thereof comprising administering to the human an effective amount of:

(a) an immunoconjugate comprising the formula

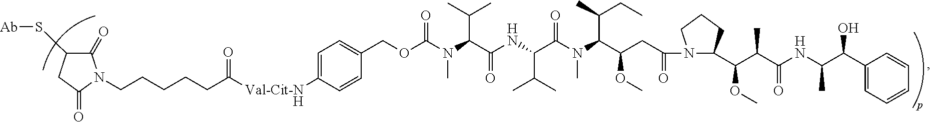

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) obinutuzumab, (c) gemcitabine, and (d) oxaliplatin.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin. In certain embodiments, p is between 2 and 5. In certain embodiments, p is 2.

In some embodiments, which may be combined with any of the preceding embodiments, the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m². In certain embodiments, the immunoconjugate, the obinutuzumab, the gemcitabine, and the oxaliplatin are administered for one or more 21-day cycles. In certain embodiments, the immunoconjugate is administered at a dose of about 1.8 mg/kg each cycle, the obinutuzumab is administered at a dose of about 1000 mg each cycle, the gemcitabine is administered at a dose of about 1000 mg/m² each cycle, and the oxaliplatin is administered at a dose of about 100 mg/m² each cycle. In certain embodiments, the obinutuzumab is administered before the immunoconjugate. In certain embodiments, the gemcitabine is administered before the oxaliplatin. In certain embodiments, the immunoconjugate, the obinutuzumab, the gemcitabine, and the oxaliplatin are administered for up to eight 21-day cycles. In certain embodiments, the immunoconjugate, the obinutuzumab, the gemcitabine, and the oxaliplatin are administered for eight 21-day cycles.

In another aspect, provided herein is a method for treating diffuse large B-cell lymphoma (DLBCL) in a human in need thereof comprising administering to the human an effective amount of:

(a) an immunoconjugate comprising the formula

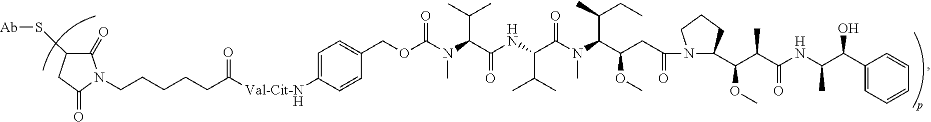

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is a method for treating diffuse large B-cell lymphoma (DLBCL) in a human in need thereof comprising administering to the human an effective amount of:

(a) an immunoconjugate comprising the formula

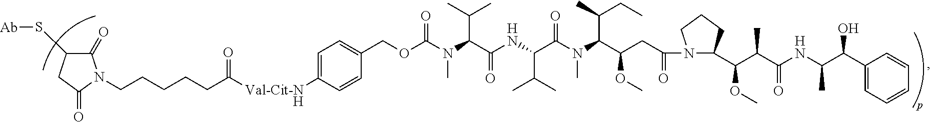

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is a method for treating diffuse large B-cell lymphoma (DLBCL) in a human in need thereof comprising administering to the human an effective amount of:

(a) an immunoconjugate comprising the formula

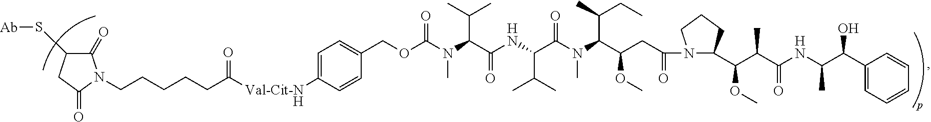

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.4 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is a method for treating diffuse large B-cell lymphoma (DLBCL) in a human in need thereof comprising administering to the human an effective amount of:

(a) an immunoconjugate comprising the formula

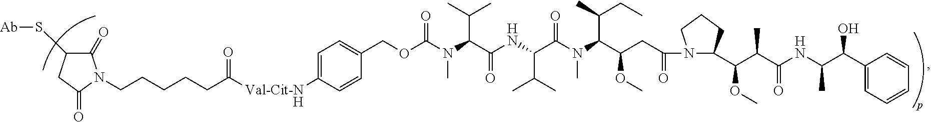

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.4 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is a method for treating diffuse large B-cell lymphoma (DLBCL) in a human in need thereof comprising administering to the human an effective amount of:

(a) an immunoconjugate comprising the formula

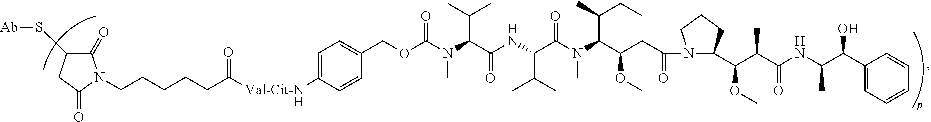

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 75 mg/m².

In another aspect, provided herein is a method for treating diffuse large B-cell lymphoma (DLBCL) in a human in need thereof comprising administering to the human an effective amount of:

(a) an immunoconjugate comprising the formula

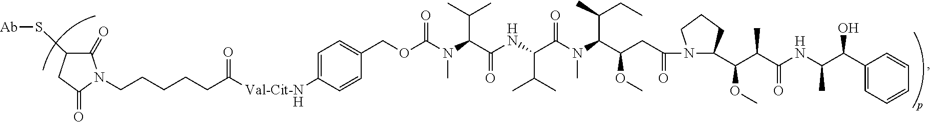

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 75 mg/m².

In another aspect, provided herein is a method for treating diffuse large B-cell lymphoma (DLBCL) in a human in need thereof comprising administering to the human an effective amount of:

(a) an immunoconjugate comprising the formula

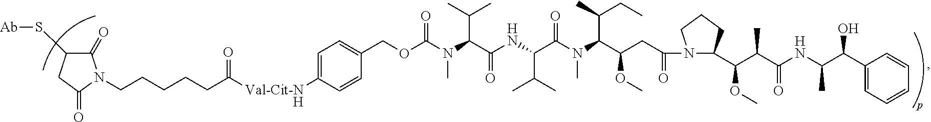

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.4 mg/kg, the obinutuzumab is administered at a dose of about 1000 mg, the gemcitabine is administered at a dose of about 1000 mg/m², and the oxaliplatin is administered at a dose of about 75 mg/m².

In another aspect, provided herein is a method for treating diffuse large B-cell lymphoma (DLBCL) in a human in need thereof comprising administering to the human an effective amount of:

(a) an immunoconjugate comprising the formula

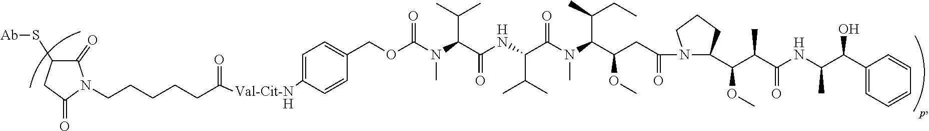

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.4 mg/kg, the obinutuzumab is administered at a dose of about 1000 mg, the gemcitabine is administered at a dose of about 1000 mg/m², and the oxaliplatin is administered at a dose of about 75 mg/m².

In some embodiments, which may be combined with any of the preceding embodiments, the immunoconjugate, the obinutuzumab, the gemcitabine, and the oxaliplatin are administered for at least two, at least three, at least four, at least five, at least six, or at least seven 21-day cycles. In some embodiments, the immunoconjugate, the obinutuzumab, the gemcitabine, and the oxaliplatin are administered for eight 21-day cycles. In some embodiments, the obinutuzumab is administered before the immunoconjugate. In some embodiments, the gemcitabine is administered before the oxaliplatin. In some embodiments, the immunoconjugate is polatuzumab vedotin-piiq.

In some embodiments, the human has received at least one prior therapy for DLBCL. In some embodiments, the human has received at least one prior systemic therapy for DLBCL. In some embodiments, the human has received at least two prior therapies for DLBCL. In certain embodiments, the DLBCL is histologically-confirmed DLBCL, not otherwise specified (NOS) or the human has a history of transformation of indolent disease to DLBCL. In certain embodiments, the DLBCL is relapsed or refractory DLBCL. In certain embodiments, the human has an Eastern Cooperative Oncology Group (ECOG) performance status of 0, 1, or 2. In certain embodiments, the human does not have a planned autologous or allogeneic stem cell transplantation (SCT). In certain embodiments, the human has not had prior therapy with a combination of gemcitabine and a platinum-based agent. In certain embodiments, the human does not have peripheral neuropathy of greater than Grade 1 according to National Cancer Institute Common Terminology Criteria for Adverse Events, Version 5.0. In certain embodiments, the human does not have primary or secondary central nervous system lymphoma. In some embodiments, the human is not a candidate for hematopoietic stem cell transplantation (HSCT) prior to administration of the immunoconjugate, the obinutuzumab, the gemcitabine, and the oxaliplatin. In some embodiments, the human is not a candidate for autologous hematopoietic stem cell transplantation (HSCT) prior to administration of the immunoconjugate, the obinutuzumab, the gemcitabine, and the oxaliplatin. In some embodiments, the human has received at least two prior therapies for DLBCL. In some embodiments, the human has not received a prior therapy with polatuzumab vedotin-piiq for DLBCL. In some embodiments, the human is an adult. In some embodiments, the human adult has relapsed or refractory diffuse large B-cell lymphoma, not otherwise specified.

In certain embodiments, after administration of the immunoconjugate, the obinutuzumab, the gemcitabine, and the oxaliplatin, the human does not experience peripheral neuropathy of Grade 3 or greater that does not resolve to Grade 1 or lower within 14 days. In certain embodiments, among a plurality of humans treated, after administration of the immunoconjugate, the obinutuzumab, the gemcitabine, and the oxaliplatin, 33% or fewer experience peripheral neuropathy of Grade 3 or greater that does not resolve to Grade 1 or lower within 14 days. In certain embodiments, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days. In some embodiments, administration of the immunoconjugate, the obinutuzumab, the gemcitabine, and the oxaliplatin does not result in peripheral neuropathy of Grade 3 or greater in the human that does not resolve to Grade 1 or lower within 14 days. In some embodiments, administration of the immunoconjugate, the obinutuzumab, the gemcitabine, and the oxaliplatin does not result in peripheral neuropathy of Grade 4 or greater in the human. In some embodiments, administration of the immunoconjugate, the obinutuzumab, the gemcitabine, and the oxaliplatin does not result in neurotoxicity of Grade 4 or greater in the human. In some embodiments, after administration of the immunoconjugate, the obinutuzumab, the gemcitabine, and the oxaliplatin, the human does not experience peripheral neuropathy of Grade 4 or greater. In some embodiments, after administration of the immunoconjugate, the obinutuzumab, the gemcitabine, and the oxaliplatin, the human does not experience neurotoxicity of Grade 4 or greater. In some embodiments, administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days. In some embodiments, administration of the immunoconjugate, the obinutuzumab, the gemcitabine, and the oxaliplatin to a plurality of humans results in less than about 40% of the humans in the plurality experiencing peripheral neuropathy of Grade 3 or greater that does not resolve to Grade 1 or lower within 14 days. In some embodiments, administration of the immunoconjugate, the obinutuzumab, the gemcitabine, and the oxaliplatin to a plurality of humans results in less than about 8% of the humans in the plurality experiencing peripheral neuropathy of Grade 3 or greater. In some embodiments, administration of the immunoconjugate, the obinutuzumab, the gemcitabine, and the oxaliplatin to a plurality of humans results in less than about 6% of the humans in the plurality experiencing peripheral neuropathy that results in discontinuation of treatment with the immunoconjugate, the obinutuzumab, the gemcitabine, and the oxaliplatin. In certain embodiments, the immunoconjugate, the obinutuzumab, the gemcitabine, and the oxaliplatin have been administered for at least four 21-day cycles.

In another aspect, provided herein is a method for treating diffuse large B-cell lymphoma (DLBCL) in a human in need thereof comprising administering to the human an effective amount of: (a) an immunoconjugate comprising the formula

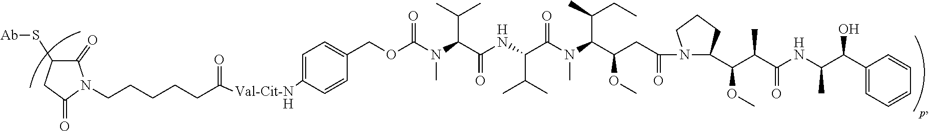

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) obinutuzumab, (c) gemcitabine, and (d) oxaliplatin; wherein, after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

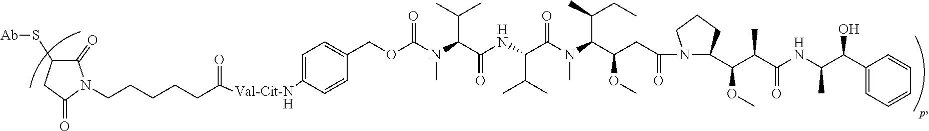

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) obinutuzumab, (c) gemcitabine, and (d) oxaliplatin; wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In another aspect, provided herein is a method for treating diffuse large B-cell lymphoma (DLBCL) in a human in need thereof comprising administering to the human an effective amount of: (a) an immunoconjugate comprising the formula

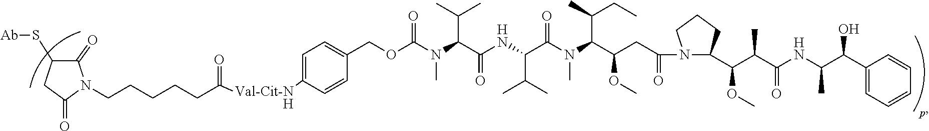

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) obinutuzumab, (c) gemcitabine, and (d) oxaliplatin; wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

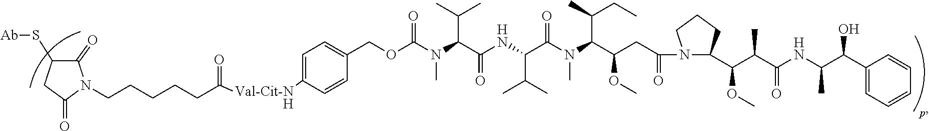

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) obinutuzumab, (c) gemcitabine, and (d) oxaliplatin; wherein, administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In certain embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In certain embodiments, p is between 2 and 5. In certain embodiments, p is between 3 and 4. In certain embodiments, the immunoconjugate is polatuzumab vedotin-piiq.

In some embodiments, which may be combined with any of the preceding embodiments, the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m². In certain embodiments, the immunoconjugate, the obinutuzumab, the gemcitabine, and the oxaliplatin are administered for one or more 21-day cycles. In certain embodiments, the immunoconjugate is administered at a dose of about 1.8 mg/kg each cycle, the obinutuzumab is administered at a dose of about 1000 mg each cycle, the gemcitabine is administered at a dose of about 1000 mg/m² each cycle, and the oxaliplatin is administered at a dose of about 100 mg/m² each cycle. In certain embodiments, the obinutuzumab is administered before the immunoconjugate. In certain embodiments, the gemcitabine is administered before the oxaliplatin. In certain embodiments, the immunoconjugate, the obinutuzumab, the gemcitabine, and the oxaliplatin are administered for up to eight 21-day cycles. In certain embodiments, the immunoconjugate, the obinutuzumab, the gemcitabine, and the oxaliplatin are administered for eight 21-day cycles.

In another aspect, provided herein is a method for treating diffuse large B-cell lymphoma (DLBCL) in a human in need thereof comprising administering to the human an effective amount of: (a) an immunoconjugate comprising the formula

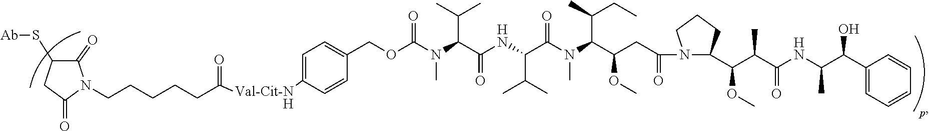

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein, after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

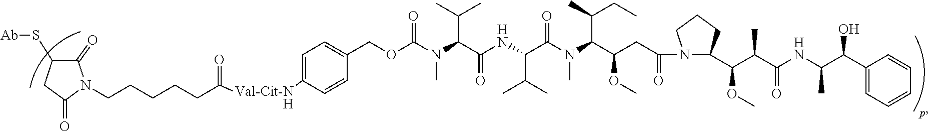

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein, after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

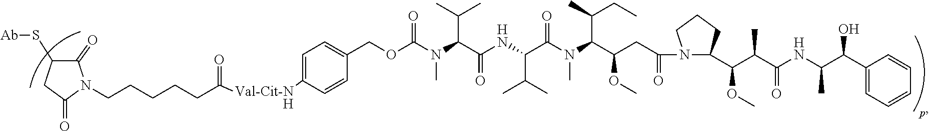

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

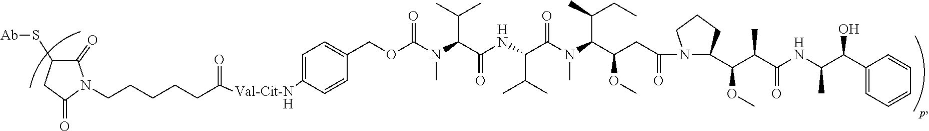

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

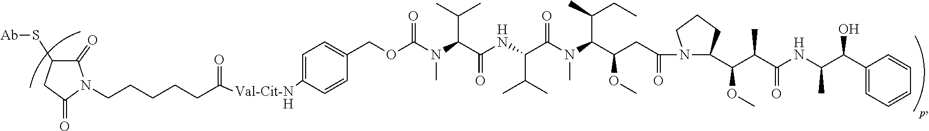

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

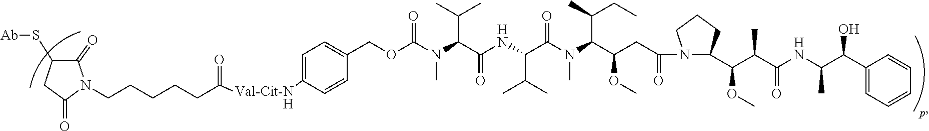

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

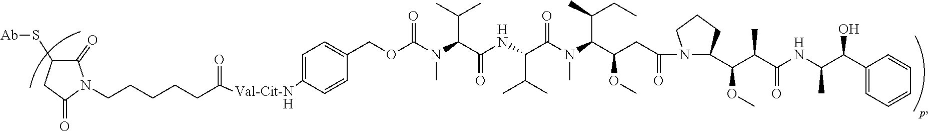

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for up to eight 21-day cycles, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is a method for treating diffuse large B-cell lymphoma (DLBCL) in a human in need thereof comprising administering to the human an effective amount of: (a) an immunoconjugate comprising the formula

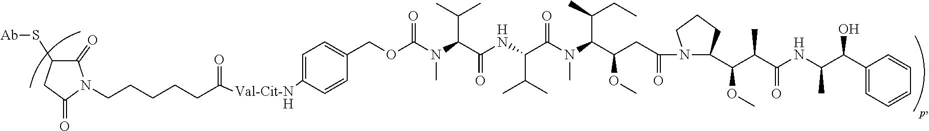

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) obinutuzumab, (c) gemcitabine, and (d) oxaliplatin; wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m²; and wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In some embodiments, which may be combined with any of the preceding embodiments, the immunoconjugate, the obinutuzumab, the gemcitabine, and the oxaliplatin have been administered for at least four 21-day cycles.

In some embodiments, which may be combined with any of the preceding embodiments, the immunoconjugate, the obinutuzumab, the gemcitabine, and the oxaliplatin are administered for at least two, at least three, at least four, at least five, at least six, or at least seven 21-day cycles. In some embodiments, the immunoconjugate, the obinutuzumab, the gemcitabine, and the oxaliplatin are administered for eight 21-day cycles. In some embodiments, the obinutuzumab is administered before the immunoconjugate. In some embodiments, the gemcitabine is administered before the oxaliplatin.

In some embodiments, which may be combined with any of the preceding embodiments, the immunoconjugate is polatuzumab vedotin-piiq.

In some embodiments, which may be combined with any of the preceding embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin. In certain embodiments, p is between 2 and 5. In certain embodiments, p is 2.

In some embodiments, the human has received at least one prior therapy for DLBCL. In some embodiments, the human has received at least one prior systemic therapy for DLBCL. In some embodiments, the human has received at least two prior therapies for DLBCL. In certain embodiments, the DLBCL is histologically-confirmed DLBCL, not otherwise specified (NOS) or the human has a history of transformation of indolent disease to DLBCL. In certain embodiments, the DLBCL is relapsed or refractory DLBCL. In certain embodiments, the human has an Eastern Cooperative Oncology Group (ECOG) performance status of 0, 1, or 2. In certain embodiments, the human does not have a planned autologous or allogeneic stem cell transplantation (SCT). In certain embodiments, the human has not had prior therapy with a combination of gemcitabine and a platinum-based agent. In certain embodiments, the human does not have peripheral neuropathy of greater than Grade 1 according to National Cancer Institute Common Terminology Criteria for Adverse Events, Version 5.0. In certain embodiments, the human does not have primary or secondary central nervous system lymphoma. In some embodiments, the human is not a candidate for hematopoietic stem cell transplantation (HSCT) prior to administration of the immunoconjugate, the obinutuzumab, the gemcitabine, and the oxaliplatin. In some embodiments, the human is not a candidate for autologous hematopoietic stem cell transplantation (HSCT) prior to administration of the immunoconjugate, the obinutuzumab, the gemcitabine, and the oxaliplatin. In some embodiments, the human has received at least two prior therapies for DLBCL. In some embodiments, the human has not received a prior therapy with polatuzumab vedotin-piiq for DLBCL. In some embodiments, the human is an adult. In some embodiments, the human adult has relapsed or refractory diffuse large B-cell lymphoma, not otherwise specified.

In some embodiments, which may be combined with any of the preceding embodiments, administration of the immunoconjugate, the obinutuzumab, the gemcitabine, and the oxaliplatin does not result in peripheral neuropathy of Grade 3 or greater in the human that does not resolve to Grade 1 or lower within 14 days. In some embodiments, administration of the immunoconjugate, the obinutuzumab, the gemcitabine, and the oxaliplatin does not result in peripheral neuropathy of Grade 4 or greater in the human. In some embodiments, administration of the immunoconjugate, the obinutuzumab, the gemcitabine, and the oxaliplatin does not result in neurotoxicity of Grade 4 or greater in the human. In some embodiments, after administration of the immunoconjugate, the obinutuzumab, the gemcitabine, and the oxaliplatin, the human does not experience peripheral neuropathy of Grade 4 or greater. In some embodiments, after administration of the immunoconjugate, the obinutuzumab, the gemcitabine, and the oxaliplatin, the human does not experience neurotoxicity of Grade 4 or greater. In some embodiments, administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days. In some embodiments, administration of the immunoconjugate, the obinutuzumab, the gemcitabine, and the oxaliplatin to a plurality of humans results in less than about 40% of the humans in the plurality experiencing peripheral neuropathy of Grade 3 or greater that does not resolve to Grade 1 or lower within 14 days. In some embodiments, administration of the immunoconjugate, the obinutuzumab, the gemcitabine, and the oxaliplatin to a plurality of humans results in less than about 8% of the humans in the plurality experiencing peripheral neuropathy of Grade 3 or greater. In some embodiments, administration of the immunoconjugate, the obinutuzumab, the gemcitabine, and the oxaliplatin to a plurality of humans results in less than about 6% of the humans in the plurality experiencing peripheral neuropathy that results in discontinuation of treatment with the immunoconjugate, the obinutuzumab, the gemcitabine, and the oxaliplatin. In some embodiments, the immunoconjugate, the obinutuzumab, the gemcitabine, and the oxaliplatin have been administered for at least four 21-day cycles. In some embodiments, administration of the immunoconjugate, the obinutuzumab, the gemcitabine, and the oxaliplatin results in a complete response in the human. In some embodiments, administration of the immunoconjugate, the obinutuzumab, the gemcitabine, and the oxaliplatin results in a partial response in the human.

In some embodiments, which may be combined with any of the preceding embodiments, administration of the immunoconjugate, the obinutuzumab, the gemcitabine, and the oxaliplatin results in progression-free survival of the human. In some embodiments, administration of the immunoconjugate, the obinutuzumab, the gemcitabine, and the oxaliplatin results in progression-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 4 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 5 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 6 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 9.5 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 11 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in progression-free survival of the human for at least about 14 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in an increase in progression-free survival of the human compared to administration of obinutuzumab, gemcitabine, and oxaliplatin to a corresponding human. In some embodiments, administration of the immunoconjugate, the obinutuzumab, the gemcitabine, and the oxaliplatin to a plurality of humans results in an increase in progression-free survival of the humans in the plurality compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin results in event-free survival of the human. In some embodiments, administration of the immunoconjugate, the obinutuzumab, the gemcitabine, and the oxaliplatin results in event-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 4 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 5 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 6 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 9.5 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 11 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in event-free survival of the human for at least about 14 months after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in an increase in event-free survival of the human compared to administration of obinutuzumab, gemcitabine, and oxaliplatin to a corresponding human. In some embodiments, administration of the immunoconjugate, the obinutuzumab, the gemcitabine, and the oxaliplatin to a plurality of humans results in an increase in event-free survival of the humans in the plurality compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin results in survival of the human for at least about 10 months or more, at least about 11 months or more, at least about 12 months or more, at least about 13 months or more, at least about 14 months or more, or at least about 15 months or more after the start of treatment with the immunoconjugate, the obinutuzumab, the gemcitabine, and the oxaliplatin. In some embodiments, administration of the immunoconjugate, the obinutuzumab, the gemcitabine, and the oxaliplatin to a plurality of humans results in a median overall survival of the humans in the plurality of at least about 10 months or more, at least about 11 months or more, at least about 12 months or more, at least about 13 months or more, at least about 14 months or more, or at least about 15 months or more after the start of treatment with the immunoconjugate, the obinutuzumab, the gemcitabine, and the oxaliplatin. In some embodiments, administration of the immunoconjugate, the obinutuzumab, the gemcitabine, and the oxaliplatin results in an increase in survival of the human compared to administration of obinutuzumab, gemcitabine, and oxaliplatin to a corresponding human. In some embodiments, administration of the immunoconjugate, the obinutuzumab, the gemcitabine, and the oxaliplatin to a plurality of humans results in an increase in the median overall survival of the humans in the plurality compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin does not result in tumor lysis syndrome in the human. In some embodiments, the methods provided herein further comprise administering a prophylactic treatment for tumor lysis syndrome to the human before, during, and/or after administration of the immunoconjugate, the obinutuzumab, the gemcitabine, and the oxaliplatin to the human.

In some embodiments, which may be combined with any of the preceding embodiments, administration of the immunoconjugate, the obinutuzumab, the gemcitabine, and the oxaliplatin does not result in an infection in the human. In some embodiments, the infection is a pneumocystis infection or a herpesvirus infection. In some embodiments, the methods provided herein further comprise administering a prophylactic treatment for infections to the human before, during, and/or after administration of the immunoconjugate, the obinutuzumab, the gemcitabine, and the oxaliplatin to the human.

In some embodiments, which may be combined with any of the preceding embodiments, administration of the immunoconjugate, the obinutuzumab, the gemcitabine, and the oxaliplatin does not result in neutropenia in the human. In some embodiments, the methods provided herein further comprise administering a prophylactic treatment for neutropenia to the human before, during, and/or after administration of the immunoconjugate, the obinutuzumab, the gemcitabine, and the oxaliplatin to the human. In some embodiments, the prophylactic treatment for neutropenia comprises administering G-CSF to the human. In some embodiments, the methods provided herein further comprise stopping treatment with the immunoconjugate, the obinutuzumab, the gemcitabine, and the oxaliplatin if Grade 3 or Grade 4 neutropenia occurs in the human after administration of the immunoconjugate, the obinutuzumab, the gemcitabine, and the oxaliplatin. In some embodiments, the methods further comprise resuming treatment with the immunoconjugate, the obinutuzumab, the gemcitabine, and the oxaliplatin if absolute neutrophil count (ANC) increases to >1000/μL in the human. In some embodiments, the methods further comprise administering one or more growth factors to treat neutropenia. In some embodiments, the one or more growth factors comprise G-CSF. In some embodiments, the methods further comprise resuming treatment with the immunoconjugate, the obinutuzumab, the gemcitabine, and the oxaliplatin without a dose reduction of the immunoconjugate, the obinutuzumab, the gemcitabine, and the oxaliplatin if ANC recovers to >1000/μL in the human on or before Day 7 of a 21-day cycle. In some embodiments, the methods further comprise resuming treatment with the immunoconjugate, the obinutuzumab, the gemcitabine, and the oxaliplatin, wherein the dose of the immunoconjugate is reduced to 1.4 mg/kg if ANC recovers to >1000/μL in the human after Day 7 of a 21-day cycle. In some embodiments, the methods further comprise discontinuing treatment with the immunoconjugate, the obinutuzumab, the gemcitabine, and the oxaliplatin if a prior dose reduction of the immunoconjugate, the obinutuzumab, the gemcitabine, and the oxaliplatin occurred.

In some embodiments, which may be combined with any of the preceding embodiments, administration of the immunoconjugate, the obinutuzumab, the gemcitabine, and the oxaliplatin does not result in Hepatitis B reactivation in the human. In some embodiments, the methods provided herein further comprise administering a prophylactic treatment for Hepatitis B reactivation to the human before, during, and/or after administration of the immunoconjugate, the obinutuzumab, the gemcitabine, and the oxaliplatin to the human. In some embodiments, the methods provided herein further comprise administering anti-viral medication to the human if Hepatitis B reactivation is detected in the human.

In some embodiments, which may be combined with any of the preceding embodiments, administration of the immunoconjugate, the obinutuzumab, the gemcitabine, and the oxaliplatin does not result in one or more adverse events selected from the group consisting of drug-induced liver injury, progressive multifocal leukoencephalopathy, systemic hypersensitivity reaction, anaphylactic reaction, anaphylactoid reaction, and second malignancy.

In some embodiments, which may be combined with any of the preceding embodiments, administration of the immunoconjugate, the obinutuzumab, the gemcitabine, and the oxaliplatin to a plurality of humans results in an increase in the complete response rate (CRR) compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in an increase in the objective response rate (ORR) compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in an increase in the best overall response rate (BOR) compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in an increase in the duration of response (DOR) compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in a 1-year progression-free survival rate of at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in a 2-year progression-free survival rate of at least about 63% or greater, at least about 65% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in a 5-year progression-free survival rate of at least about 14% or greater, at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in an increase in the 1-year, 2-year, 3.5-year, or 5-year progression-free survival rate compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in a 1-year overall survival rate of at least about 42% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in a 2-year overall survival rate of at least about 67% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in a 3.5-year overall survival rate of at least about 38% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in a 5-year overall survival rate of at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in an increase in the 1-year, 2-year, 3.5-year, or 5-year overall survival rate compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in a 2-year event-free survival rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the obinutuzumab, the gemcitabine, and the oxaliplatin to a plurality of humans results in an increase in the 1-year, 2-year, 3.5-year, or 5-year event-free survival rate compared to a corresponding plurality of humans administered obinutuzumab, gemcitabine, and oxaliplatin.

In some embodiments, which may be combined with any of the preceding embodiments, administration of the immunoconjugate, the obinutuzumab, the gemcitabine, and the oxaliplatin to a plurality of humans results in an objective response rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality.

In some embodiments, which may be combined with any of the preceding embodiments, administration of the immunoconjugate, the obinutuzumab, the gemcitabine, and the oxaliplatin to a plurality of humans results in a complete response rate of at least about 35% or greater, at least about 40% or greater, at least about 50% or greater, at least about 60% or greater, at least about 70% or greater, at least about 80% or greater, at least about 90% or greater, at least about 95% or greater, or about 100% of the humans in the plurality.

In some embodiments, which may be combined with any of the preceding embodiments, administration of the immunoconjugate, the obinutuzumab, the gemcitabine, and the oxaliplatin to a plurality of humans results in a partial response rate of at least about 10% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, which may be combined with any of the preceding embodiments, administration of the immunoconjugate, the obinutuzumab, the gemcitabine, and the oxaliplatin to a plurality of humans results in an increase in the partial response rate compared to a corresponding plurality of humans administered obinutuzumab, gemcitabine, and oxaliplatin.

In another aspect, provided herein is a kit comprising an immunoconjugate comprising the formula

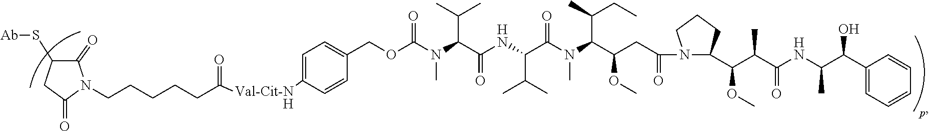

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with obinutuzumab, gemcitabine, and oxaliplatin for treating a human in need thereof having diffuse large B-cell lymphoma (DLBCL) according to any of the preceding embodiments.

In another aspect, provided herein is a kit comprising polatuzumab vedotin-piiq for use in combination with obinutuzumab, gemcitabine, and oxaliplatin for treating a human in need thereof having diffuse large B-cell lymphoma (DLBCL) according to according to any of the preceding embodiments.

In another aspect, provided herein is an immunoconjugate comprising the formula

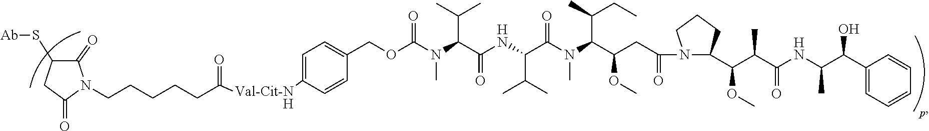

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments.

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

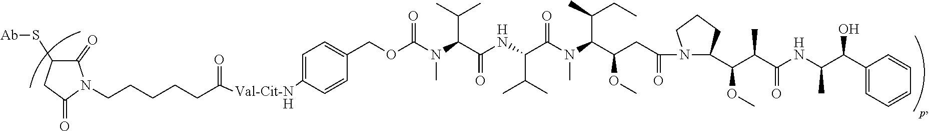

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

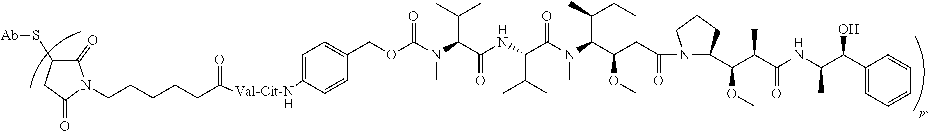

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

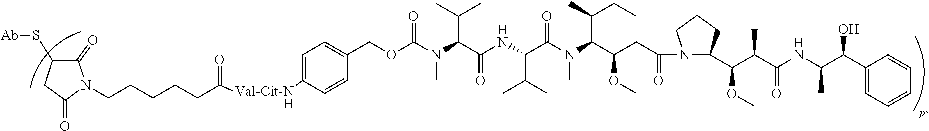

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8.

In another aspect, provided herein is an immunoconjugate comprising the formula

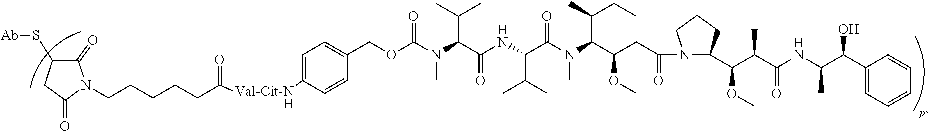

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is an immunoconjugate comprising the formula

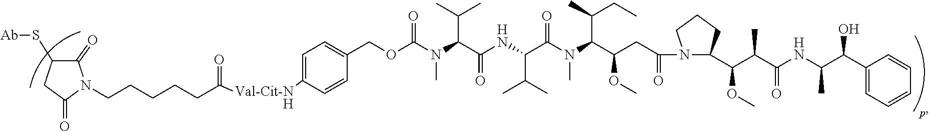

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is an immunoconjugate comprising the formula

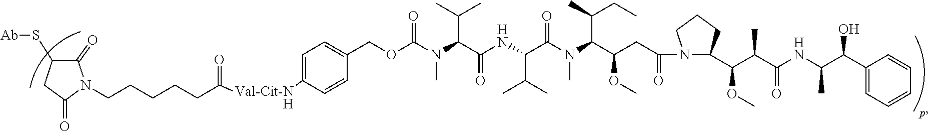

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is an immunoconjugate comprising the formula

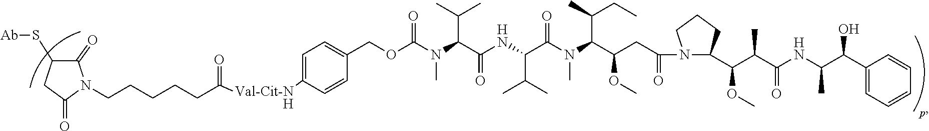

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

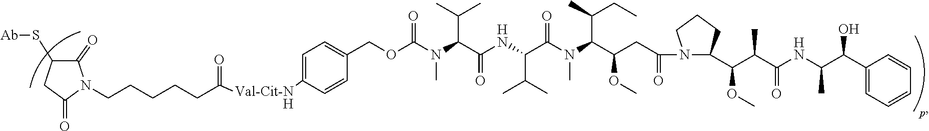

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

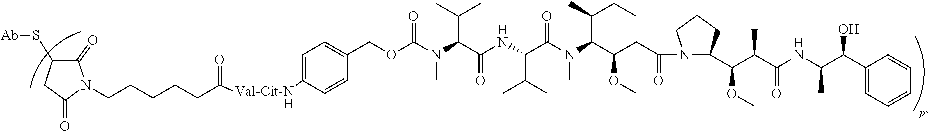

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

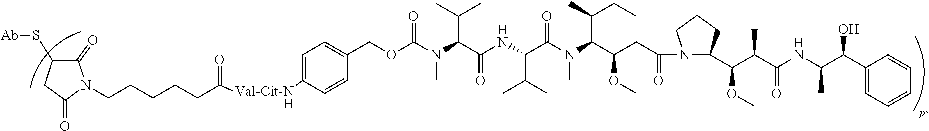

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

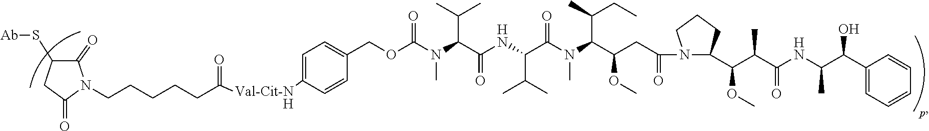

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

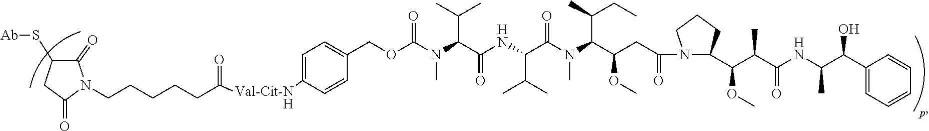

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

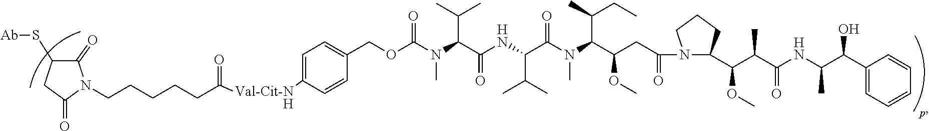

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

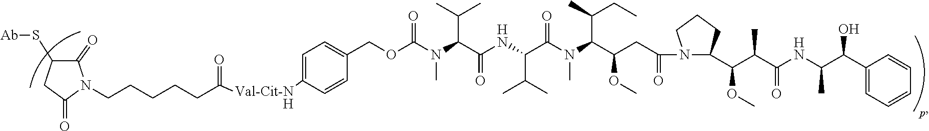

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

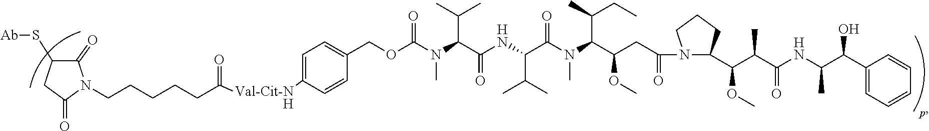

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

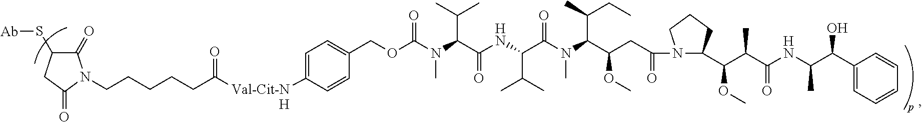

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, after administration of the immunoconjugate, the obinutuzumab, the gemcitabine, and the oxaliplatin, the human does not experience peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

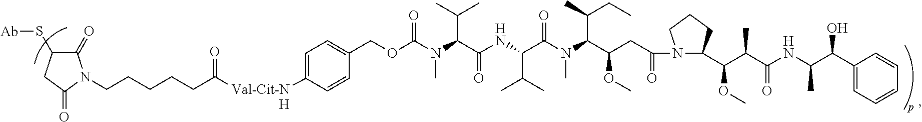

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

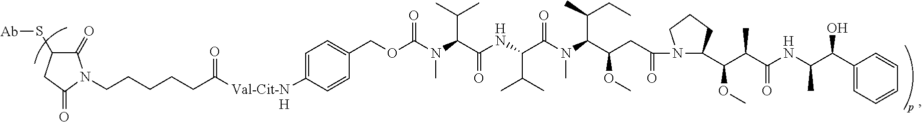

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days.

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

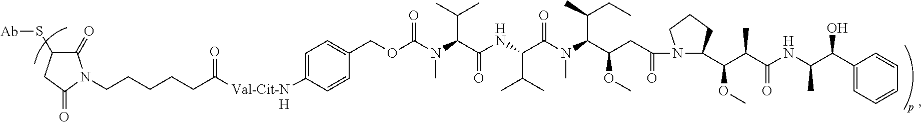

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

In another aspect, provided herein a method for treating relapsed or refractory diffuse large B-cell lymphoma (R/R DLBCL) in a human in need thereof, comprising administering to the human polatuzumab vedotin-piiq at a dose of 1.8 mg/kg, rituximab at a dose of 375 mg/m², gemcitabine at a dose of 1000 mg/m², and oxaliplatin at a dose of 100 mg/m².

In another aspect, provided herein a method for treating relapsed or refractory diffuse large B-cell lymphoma (R/R DLBCL) in a human in need thereof, comprising administering to the human polatuzumab vedotin-piiq at a dose of 1.8 mg/kg, rituximab at a dose of 375 mg/m², gemcitabine at a dose of 1000 mg/m², and oxaliplatin at a dose of 100 mg/m², wherein the polatuzumab vedotin-piiq, the rituximab, the gemcitabine, and the oxaliplatin are administered for at least one 21-day cycle, wherein the polatuzumab vedotin-piiq and the rituximab are administered intravenously on Day 1 of each 21-day cycle, and wherein the gemcitabine and the oxaliplatin are administered intravenously on Day 2 of each 21-day cycle.

In another aspect, provided herein a method for treating relapsed or refractory diffuse large B-cell lymphoma (R/R DLBCL) in a human in need thereof, comprising administering to the human iladatuzumab vedotin at a dose of 4.8 mg/kg, rituximab at a dose of 375 mg/m², gemcitabine at a dose of 1000 mg/m², and oxaliplatin at a dose of 100 mg/m².

In another aspect, provided herein is a method for treating relapsed or refractory diffuse large B-cell lymphoma (R/R DLBCL) in a human in need thereof, comprising administering to the human iladatuzumab vedotin at a dose of 3.6 mg/kg, rituximab at a dose of 375 mg/m², gemcitabine at a dose of 1000 mg/m², and oxaliplatin at a dose of 100 mg/m².

In another aspect, provided herein a method for treating relapsed or refractory diffuse large B-cell lymphoma (R/R DLBCL) in a human in need thereof, comprising administering to the human iladatuzumab vedotin at a dose of 4.8 mg/kg, rituximab at a dose of 375 mg/m², gemcitabine at a dose of 1000 mg/m², and oxaliplatin at a dose of 100 mg/m², wherein the iladatuzumab vedotin, the rituximab, the gemcitabine, and the oxaliplatin are administered for at least one 21-day cycle, wherein the iladatuzumab vedotin and the rituximab are administered intravenously on Day 1 of each 21-day cycle, and wherein the gemcitabine and the oxaliplatin are administered intravenously on Day 2 of each 21-day cycle.

In another aspect, provided herein a method for treating relapsed or refractory diffuse large B-cell lymphoma (R/R DLBCL) in a human in need thereof, comprising administering to the human iladatuzumab vedotin at a dose of 3.6 mg/kg, rituximab at a dose of 375 mg/m², gemcitabine at a dose of 1000 mg/m², and oxaliplatin at a dose of 100 mg/m², wherein the iladatuzumab vedotin, the rituximab, the gemcitabine, and the oxaliplatin are administered for at least one 21-day cycle, wherein the iladatuzumab vedotin and the rituximab are administered intravenously on Day 1 of each 21-day cycle, and wherein the gemcitabine and the oxaliplatin are administered intravenously on Day 2 of each 21-day cycle.

In another aspect, provided herein a method for treating relapsed or refractory diffuse large B-cell lymphoma (R/R DLBCL) in a human in need thereof, comprising administering to the human polatuzumab vedotin-piiq at a dose of 1.8 mg/kg, obinutuzumab at a dose of 1000 mg, gemcitabine at a dose of 1000 mg/m², and oxaliplatin at a dose of 100 mg/m².

In another aspect, provided herein a method for treating relapsed or refractory diffuse large B-cell lymphoma (R/R DLBCL) in a human in need thereof, comprising administering to the human polatuzumab vedotin-piiq at a dose of 1.8 mg/kg, obinutuzumab at a dose of 1000 mg, gemcitabine at a dose of 1000 mg/m², and oxaliplatin at a dose of 100 mg/m², wherein the polatuzumab vedotin-piiq, the obinutuzumab, the gemcitabine, and the oxaliplatin are administered for at least one 21-day cycle, wherein the polatuzumab vedotin-piiq and the obinutuzumab are administered intravenously on Day 1 of each 21-day cycle, and wherein the gemcitabine and the oxaliplatin are administered intravenously on Day 2 of each 21-day cycle.

In another aspect, provided herein a method for treating relapsed or refractory diffuse large B-cell lymphoma (R/R DLBCL) in a human in need thereof, comprising administering to the human iladatuzumab vedotin at a dose of 4.8 mg/kg, obinutuzumab at a dose of 1000 mg, gemcitabine at a dose of 1000 mg/m², and oxaliplatin at a dose of 100 mg/m².

In another aspect, provided herein is a method for treating relapsed or refractory diffuse large B-cell lymphoma (R/R DLBCL) in a human in need thereof, comprising administering to the human iladatuzumab vedotin at a dose of 3.6 mg/kg, obinutuzumab at a dose of 1000 mg, gemcitabine at a dose of 1000 mg/m², and oxaliplatin at a dose of 100 mg/m².

In another aspect, provided herein a method for treating relapsed or refractory diffuse large B-cell lymphoma (R/R DLBCL) in a human in need thereof, comprising administering to the human iladatuzumab vedotin at a dose of 4.8 mg/kg, obinutuzumab at a dose of 1000 mg, gemcitabine at a dose of 1000 mg/m², and oxaliplatin at a dose of 100 mg/m², wherein the iladatuzumab vedotin, the obinutuzumab, the gemcitabine, and the oxaliplatin are administered for at least one 21-day cycle, wherein the iladatuzumab vedotin and the obinutuzumab are administered intravenously on Day 1 of each 21-day cycle, and wherein the gemcitabine and the oxaliplatin are administered intravenously on Day 2 of each 21-day cycle.

In another aspect, provided herein a method for treating relapsed or refractory diffuse large B-cell lymphoma (R/R DLBCL) in a human in need thereof, comprising administering to the human iladatuzumab vedotin at a dose of 3.6 mg/kg, obinutuzumab at a dose of 1000 mg, gemcitabine at a dose of 1000 mg/m², and oxaliplatin at a dose of 100 mg/m², wherein the iladatuzumab vedotin, the obinutuzumab, the gemcitabine, and the oxaliplatin are administered for at least one 21-day cycle, wherein the iladatuzumab vedotin and the obinutuzumab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

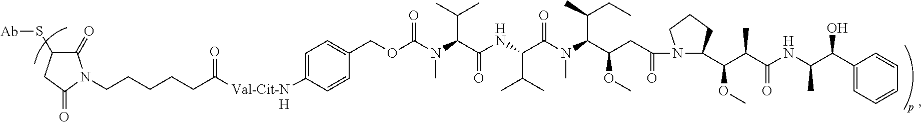

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

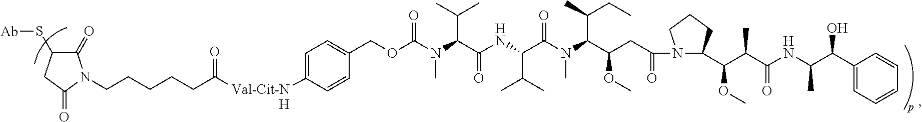

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

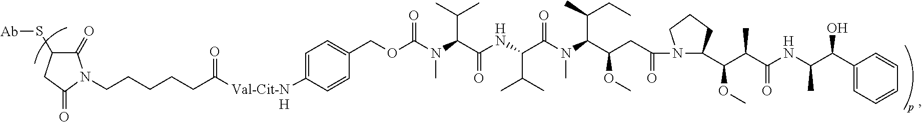

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

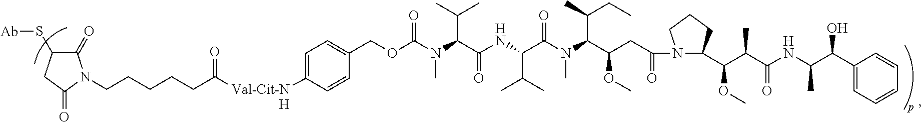

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

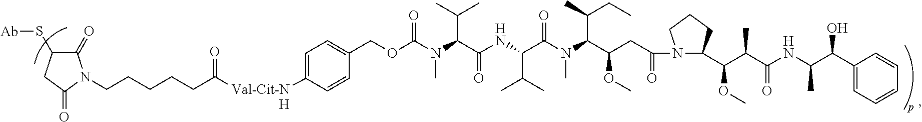

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein after administration of the immunoconjugate, the rituximab, the gemcitabine, and the oxaliplatin, the human does not experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

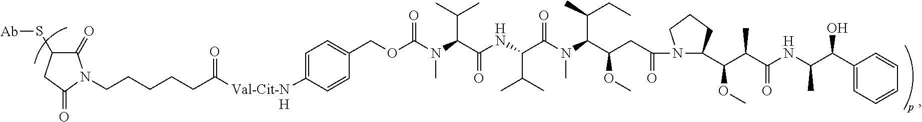

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein administration of the immunoconjugate, the rituximab, the gemcitabine, and the oxaliplatin does not result in peripheral neuropathy of Grade 3 or greater in the human that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

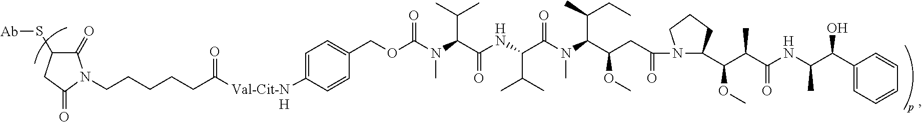

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

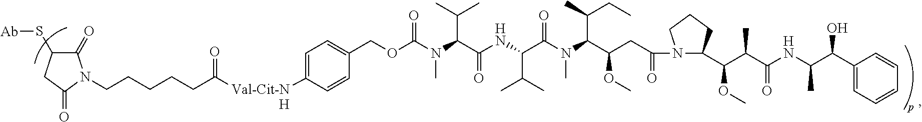

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with rituximab, gemcitabine, and oxaliplatin according to any of the preceding embodiments, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

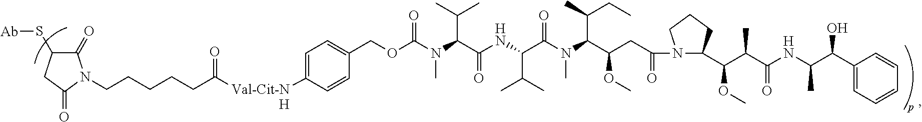

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the rituximab, the gemcitabine, and the oxaliplatin, the human does not experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

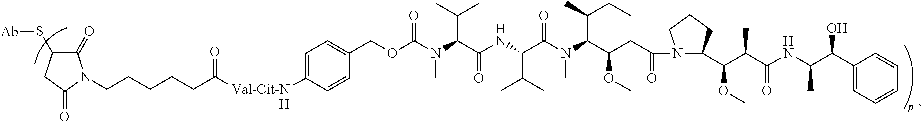

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

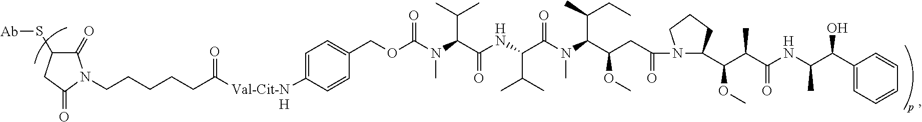

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is rituximab for use in a method of treating diffuse large B-cell lymphoma (DLBCL) in combination with an immunoconjugate, gemcitabine, and oxaliplatin according to any of the preceding embodiments, wherein the immunoconjugate comprises the formula

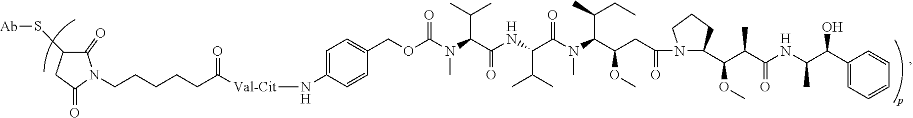

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

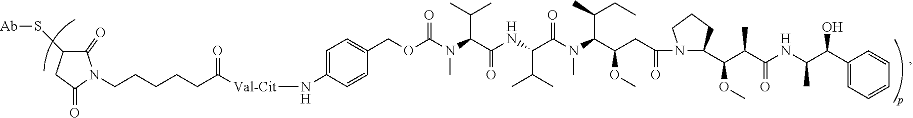

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the rituximab, the gemcitabine, and the oxaliplatin, the human does not experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

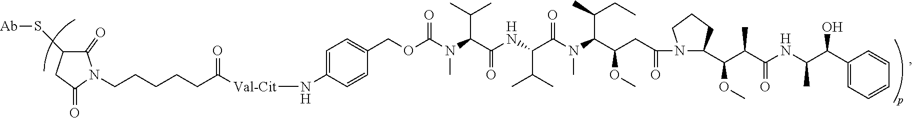

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

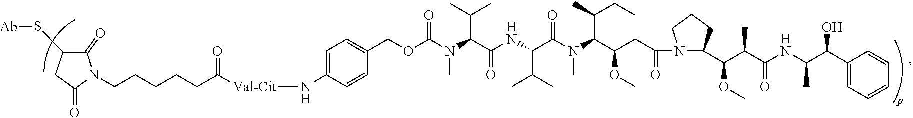

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is gemcitabine for use in a method of treating diffuse large B-cell lymphoma (DLBCL) in combination with an immunoconjugate, rituximab, and oxaliplatin according to any of the preceding embodiments, wherein the immunoconjugate comprises the formula

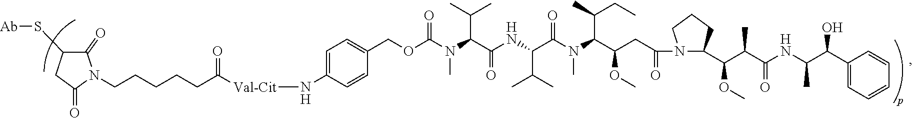

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

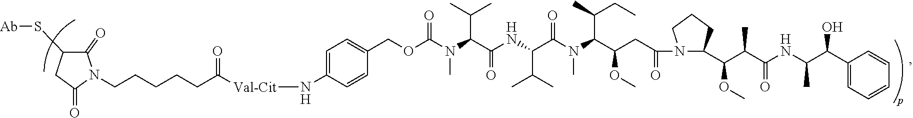

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, after administration of the immunoconjugate, the rituximab, the gemcitabine, and the oxaliplatin, the human does not experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

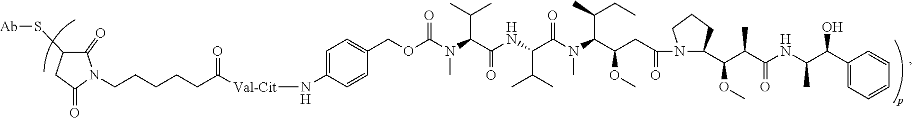

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin does not result in peripheral neuropathy of Grade 3 or greater in the human that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

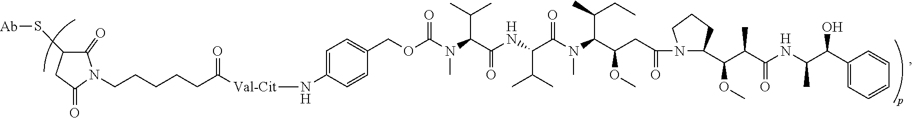

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the rituximab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is oxaliplatin for use in a method of treating diffuse large B-cell lymphoma (DLBCL) in combination with an immunoconjugate, gemcitabine, and rituximab according to any of the preceding embodiments, wherein the immunoconjugate comprises the formula

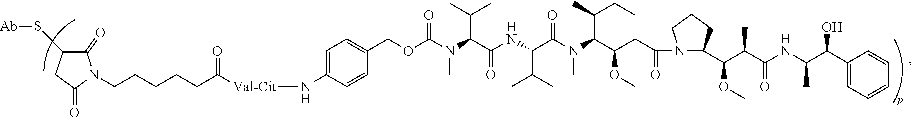

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate and the rituximab are administered intravenously on Day 1 of each 21-day cycle, and wherein the gemcitabine and the oxaliplatin are administered intravenously on Day 2 of each 21-day cycle.

In another aspect, provided herein is an immunoconjugate comprising the formula

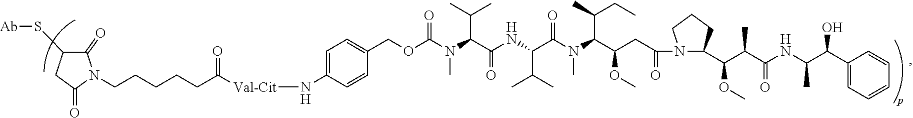

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

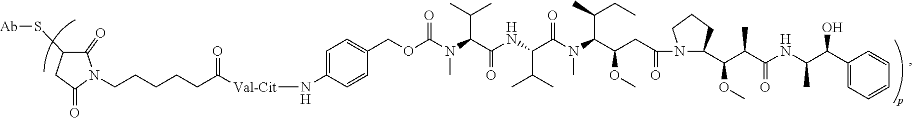

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

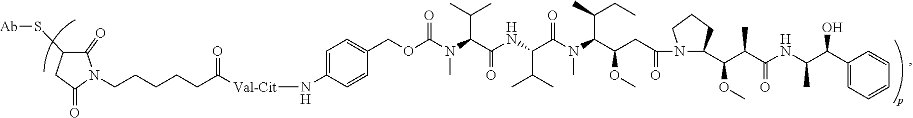

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

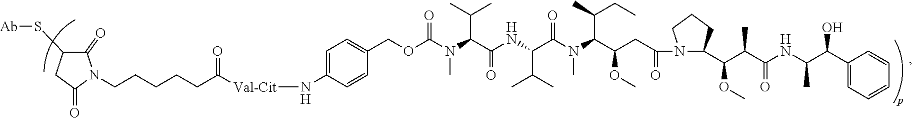

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is an immunoconjugate comprising the formula

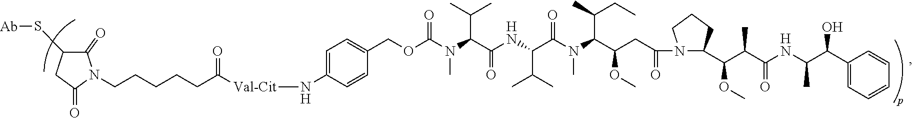

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is an immunoconjugate comprising the formula

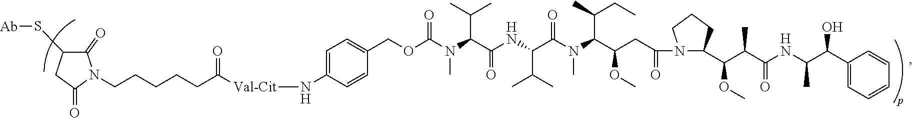

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein administration of the immunoconjugate, the obinutuzumab, the gemcitabine, and the oxaliplatin does not result in peripheral neuropathy of Grade 3 or greater in the human that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is an immunoconjugate comprising the formula

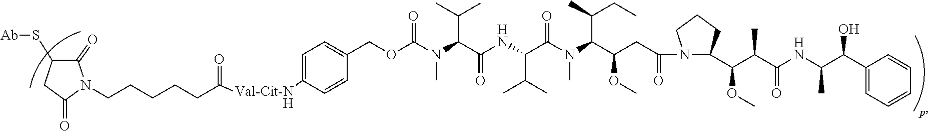

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is an immunoconjugate comprising the formula

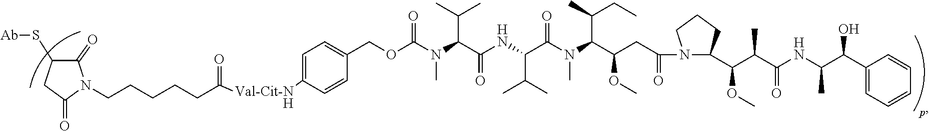

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL) in combination with obinutuzumab, gemcitabine, and oxaliplatin according to any of the preceding embodiments, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

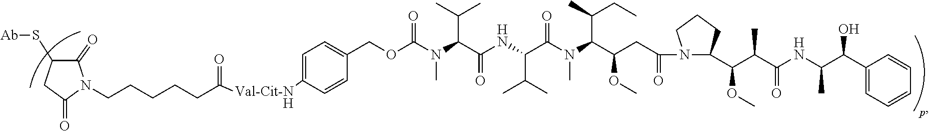

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

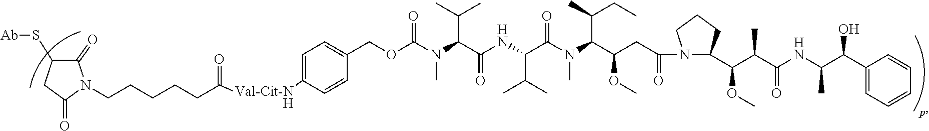

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

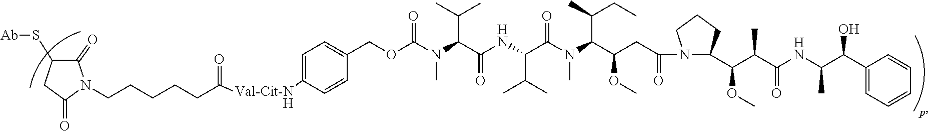

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is obinutuzumab for use in a method of treating diffuse large B-cell lymphoma (DLBCL) in combination with an immunoconjugate, gemcitabine, and oxaliplatin according to any of the preceding embodiments, wherein the immunoconjugate comprises the formula

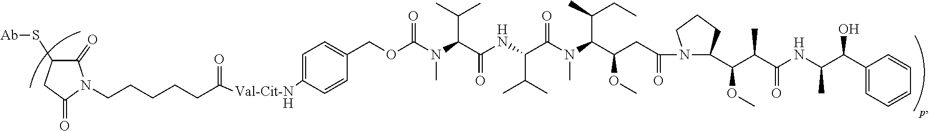

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

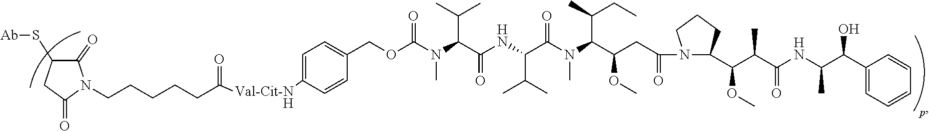

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein after administration of the immunoconjugate, the obinutuzumab, the gemcitabine, and the oxaliplatin, the human does not experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

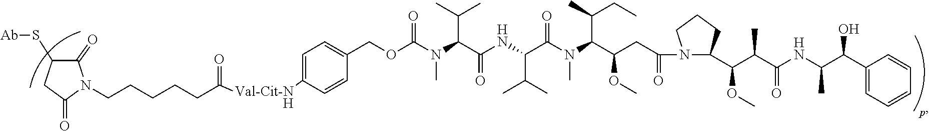

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

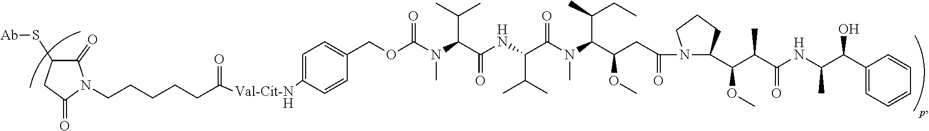

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is gemcitabine for use in a method of treating diffuse large B-cell lymphoma (DLBCL) in combination with an immunoconjugate, obinutuzumab, and oxaliplatin according to any of the preceding embodiments, wherein the immunoconjugate comprises the formula

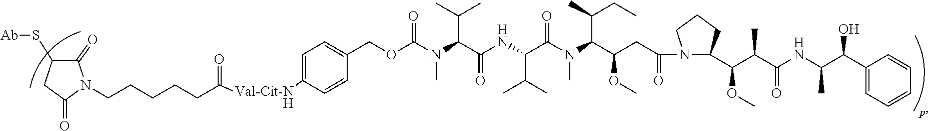

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

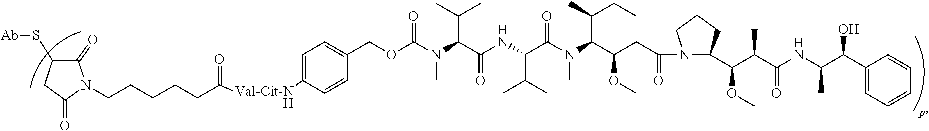

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, after administration of the immunoconjugate, the obinutuzumab, the gemcitabine, and the oxaliplatin, the human does not experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

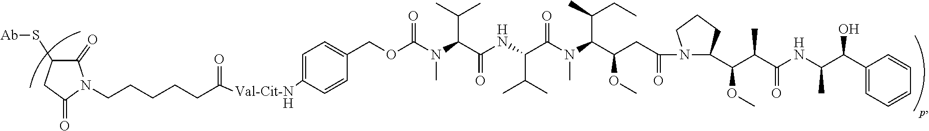

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin does not result in peripheral neuropathy of Grade 3 or greater in the human that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

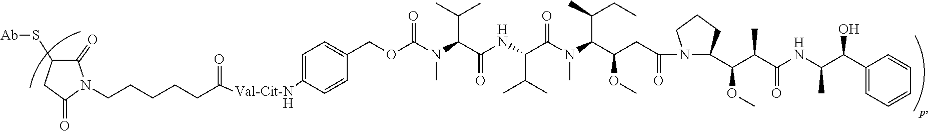

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein, among a plurality of humans treated, after administration of the immunoconjugate, the obinutuzumab, the gemcitabine, and the oxaliplatin, 33% or fewer of the humans in the plurality experience peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

In another aspect, provided herein is oxaliplatin for use in a method of treating diffuse large B-cell lymphoma (DLBCL) in combination with an immunoconjugate, gemcitabine, and obinutuzumab according to any of the preceding embodiments, wherein the immunoconjugate comprises the formula

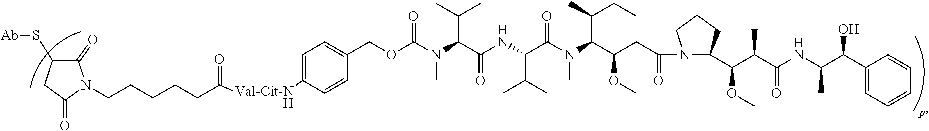

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, wherein administration of the immunoconjugate, the obinutuzumab, the gemcitabine, and the oxaliplatin to a plurality of humans results in 33% or fewer of the humans in the plurality experiencing peripheral neuropathy of Grade 3 or greater that does not resolve to Grade 1 or lower within 14 days; and wherein the immunoconjugate, the obinutuzumab, the gemcitabine, and the oxaliplatin are administered for at least one 21-day cycle, wherein during each cycle the immunoconjugate is administered at a dose of about 1.8 mg/kg, the obinutuzumab is administered at a dose of about 1000 mg, the gemcitabine is administered at a dose of about 1000 mg/m², and the oxaliplatin is administered at a dose of about 100 mg/m².

### BRIEF DESCRIPTION OF THE DRAWINGS

FIG. 1 is a schematic of the overall design of the study described in Example 1. Pola=polatuzumab vedotin; Rand=randomize; R-GemOx=rituximab plus gemcitabine plus oxaliplatin; R/R DLBCL=relapsed or refractory diffuse large B-cell lymphoma.

FIG. 2 is a schematic of the Stage 1 (Safety Run-in) of the study described in Example 1. EOT=end of treatment; RCT=randomized controlled trial (Stage 2).

FIGS. 3A-3B show the experimental treatment and control treatment regimens of the study described in Example 1. FIG. 3A is a schematic of the experimental treatment regimen (Pola-R-GemOx) of the study described in Example 1. Diamonds represent doses of rituximab (375 mg/m²); circles represent doses of polatuzumab vedotin (1.8 mg/kg); thin arrows represent doses of gemcitabine (1000 mg/m²); wide arrows represent doses of oxaliplatin (100 mg/m²); ^{a}rituximab is administered before polatuzumab vedotin; ^{b} gemcitabine is administered before oxaliplatin. FIG. 3B is a schematic of the control treatment regimen (R-GemOx) of the study described in Example 1. Diamonds represent doses of rituximab (375 mg/m²); thin arrows represent doses of gemcitabine (1000 mg/m²); wide arrows represent doses of oxaliplatin (100 mg/m²); ^{a}gemcitabine is administered before oxaliplatin. In FIGS. 3A-3B, C=cycle (1 cycle is 21 days; D=day; and IV=intravenous.

### DETAILED DESCRIPTION

As used herein, the term “polatuzumab vedotin-piiq” refers to an anti-CD79b immunoconjugate having the IUPHAR/BPS Number 8404, the KEGG Number D10761, or the CAS Registry Number 1313206-42-6. Polatuzumab vedotin-piiq is also interchangeably referred to as “polatuzumab vedotin”, “huMA79bv28-MC-vc-PAB-MMAE”, “DCDS4501A”, or “RG7596.”

Provided herein are methods for treating or delaying progression of diffuse large B-cell lymphoma (DLBCL, e.g., relapsed/refractory DLBCL) in an individual (e.g., a human) comprising administering to the individual an effective amount of an anti-CD79b immunoconjugate (e.g., polatuzumab vedotin-piiq), an anti-CD20 agent (e.g., an anti-CD20 antibody such as rituximab), and one or more chemotherapeutic agents (e.g., gemcitabine and oxaliplatin).

In some embodiments, the method comprises treating an individual having diffuse large B-cell lymphoma (DLBCL), e.g., relapsed/refractory DLBCL, by administering to the individual (a) an immunoconjugate comprising the formula

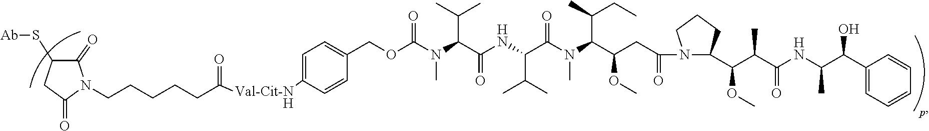

wherein Ab is an anti-CD79b antibody comprising (i) an HVR-H1 that comprises the amino acid sequence of GYTFSSYWIE (SEQ ID NO: 21); (ii) an HVR-H2 comprising the amino acid sequence of GEILPGGGDTNYNEIFKG (SEQ ID NO: 22); (iii) an HVR-H3 comprising the amino acid sequence of TRRVPIRLDY (SEQ ID NO: 23); (iv) an HVR-L1 comprising the amino acid sequence of KASQSVDYEGDSFLN (SEQ ID NO: 24); (v) an HVR-L2 comprising the amino acid sequence of AASNLES (SEQ ID NO: 25); and (vi) an HVR-L3 comprising the amino acid sequence of QQSNEDPLT (SEQ ID NO:26), and wherein p is between 1 and 8 (e.g., between 2 and 5, or between 3 and 4), (b) an anti-CD20 agent (e.g., rituximab), and (c) one or more chemotherapeutic agents (e.g., gemcitabine and oxaliplatin). In some embodiments, the immunoconjugate is administered at a dose between about 1.4 mg/kg and about 1.8 mg/kg, the anti-CD20 agent (e.g., rituximab) is administered at a dose of about 375 mg/m², the gemcitabine is administered at a dose of about 1000 mg/m², and the oxaliplatin is administered at a dose of about 100 mg/m².

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “CD79b,” as used herein, refers to any native CD79b from any vertebrate source, including mammals such as primates (e.g., humans, cynomologus monkey (“cyno”)) and rodents (e.g., mice and rats), unless otherwise indicated. Human CD79b is also referred herein to as “Igβ,” “B29,” “DNA225786,” or “PRO36249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b, as well as any form of CD79b that results from processing in the cell. The term also encompasses naturally occurring variants of CD79b, e.g., splice variants, allelic variants, and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally-occurring truncated or secreted forms of the specific CD79b polypeptide (e.g., an extracellular domain sequence), naturally-occurring variant forms (e.g., alternatively spliced forms) and naturally-occurring allelic variants of the polypeptide.

“CD20” as used herein refers to the human B-lymphocyte antigen CD20 (also known as CD20, B-lymphocyte surface antigen B1, Leu-16, Bp35, BMS, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms, and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “epitope” refers to the particular site on an antigen molecule to which an antibody binds.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, anti-CD79b antibody binds to an epitope of CD79b that is conserved among CD79b from different species.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM. In certain embodiments, anti-CD20 antibody binds to an epitope of CD20 that is conserved among CD20 from different species.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007). The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

“Isolated nucleic acid encoding an anti-CD79b antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHL CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

“CD79b polypeptide variant” means a CD79b polypeptide, preferably an active CD79b polypeptide, as defined herein having at least about 80% amino acid sequence identity with a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other fragment of a full-length CD79b polypeptide sequence as disclosed herein (such as those encoded by a nucleic acid that represents only a portion of the complete coding sequence for a full-length CD79b polypeptide). Such CD79b polypeptide variants include, for instance, CD79b polypeptides wherein one or more amino acid residues are added, or deleted, at the N- or C-terminus of the full-length native amino acid sequence. Ordinarily, a CD79b polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other specifically defined fragment of a full-length CD79b polypeptide sequence as disclosed herein. Ordinarily, CD79b variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, CD79b variant polypeptides will have no more than one conservative amino acid substitution as compared to the native CD79b polypeptide sequence, alternatively no more than 2, 3, 4, 5, 6, 7, 8, 9, or 10 conservative amino acid substitution as compared to the native CD79b polypeptide sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

wherein X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including but not limited to a cytotoxic agent.

In the context of the formulas provided herein, “p” refers to the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4, (e.g., about 3.5).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The terms “cancer” and “cancerous” refer to or describe the physiological condition in mammals that is typically characterized by unregulated cell growth. Examples of cancer include but are not limited to, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), follicular lymphoma (e.g., relapsed/refractory follicular lymphoma) intermediate grade diffuse NHL, diffuse large B-cell lymphoma (DLBCL), relapsed DLBCL, refractory DLBCL, relapsed/refractory DLBCL, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, reduction of free light chain, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, the antibodies described herein are used to delay development of a disease or to slow the progression of a disease.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

As used herein, “in conjunction with” or “in combination with” refer to administration of one treatment modality in addition to another treatment modality. As such, “in conjunction with” or “in combination with” refer to administration of one treatment modality before, during, or after administration of the other treatment modality to the individual.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, Astra7eneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin ylI and calicheamicin wlI (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, everolimus, sotrataurin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional examples include of chemotherapeutic agents include bendamustine (or bendamustine-HCl) (TREANDA®), ibrutinib, lenalidomide, and/or idelalisib (GS-1101).

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVIS TAO), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARDO), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine.

In some embodiments, the chemotherapeutic agent includes topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

Chemotherapetuic agent also includes antibodies such as alemtuzumab (Campath), bevacizumab (AVAS TIN®, Genentech); cetuximab (ERBITUX®, Imclone); panitumumab (VECTIBIX®, Amgen), rituximab (RITUXAN®, Genentech/Biogen Idec), ublituximab, ofatumumab, ibritumomab tiuxetan, pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

The term “package insert” is used to refer to instructions customarily included in commercial packages of therapeutic products, that contain information about the indications, usage, dosage, administration, combination therapy, contraindications and/or warnings concerning the use of such therapeutic products.

“Alkyl” is C₁-C₁₈ hydrocarbon containing normal, secondary, tertiary, or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, —methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl,-acetylenyl, -propynyl, -1-butynyl, -2-butynyl, -1-pentynyl, -2-pentynyl, -3-methyl-1 butynyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, -and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₅ alkoxy” is an alkoxy group with 1 to 5 carbon atoms. Alkoxy groups may can be unsubstituted or substituted with one or more groups, as described above for alkyl groups.

“Alkenyl” is C2-C18 hydrocarbon containing normal, secondary, tertiary, or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H7), and 5-hexenyl (—CH₂CH₂CH₂CH₂CH═CH₂). A “C₂-C₈ alkenyl” is a hydrocarbon containing 2 to 8 normal, secondary, tertiary, or cyclic carbon atoms with at least one site of unsaturation, i.e. a carbon-carbon, sp² double bond.

“Alkynyl” is C2-C18 hydrocarbon containing normal, secondary, tertiary, or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₈ alkynyl” is a hydrocarbon containing 2 to 8 normal, secondary, tertiary or, cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene, and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl, and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₅-C₂₀ aryl” is an aryl group with 5 to 20 carbon atoms in the carbocyclic aromatic rings. Examples of C₅-C₂₀ aryl groups include, but are not limited to, phenyl, naphthyl, and anthracenyl. A C₅-C₂₀ aryl group can be substituted or unsubstituted as described above for aryl groups. A “C₅-C₁₄ aryl” is an aryl group with 5 to 14 carbon atoms in the carbocyclic aromatic rings. Examples of C₅-C₁₄ aryl groups include, but are not limited to, phenyl, naphthyl, and anthracenyl. A C₅-C₁₄ aryl group can be substituted or unsubstituted as described above for aryl groups.

An “arylene” is an aryl group which has two covalent bonds and can be in the ortho, meta, or para configurations as shown in the following structures:

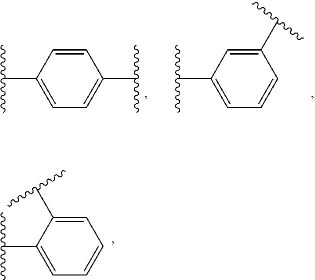

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Exemplary heterocycles are described, e.g., in Paquette, Leo A., “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃-C₂₀ heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)nO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, or iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III. Methods

Provided herein are methods of treating a B-cell proliferative disorder (such as diffuse large B-cell lymphoma (DLBCL), e.g., relapsed/refractory DLBCL) in an individual (e.g., a human individual) in need thereof comprising administering to the individual an effective amount of (a) an immunoconjugate comprising an antibody which binds CD79b linked to a cytotoxic agent, (b) an anti-CD20 antibody, and (c) one or more chemotherapeutic agents.

In some embodiments, the anti-CD79b immunoconjugate is polatuzumab vedotin-piiq. In some embodiments, the anti-CD20 antibody is rituximab. In some embodiments, the one or more chemotherapeutic agents comprise gemcitabine. In some embodiments, the one or more chemotherapeutic agents comprise oxaliplatin. In some embodiments, the one or more chemotherapeutic agents are gemcitabine and oxaliplatin.

The terms “co-administration” or “co-administering” refer to the administration of the anti-CD79b immunoconjugate, the anti-CD20 antibody, and the one or more chemotherapeutic agents as two (or more) separate formulations (or as one single formulation comprising the anti-CD79b immunoconjugate, the anti-CD20 antibody, and the one or more chemotherapeutic agents). Where separate formulations are used, the co-administration can be simultaneous or sequential in any order, wherein preferably there is a time period while all active agents simultaneously exert their biological activities. In some embodiments, the anti-CD79b immunoconjugate, the anti-CD20 antibody, and the one or more chemotherapeutic agents are co-administered either simultaneously or sequentially. In some embodiments, when all therapeutic agents are co-administered sequentially, the dose is administered either on the same day in two or more separate administrations, or one or more of the agents is administered on day 1 (e.g., on day 1 of a 21-day cycle), the other agent(s) are co-administered on about day 2 (e.g., on day 2 of a 21-day cycle). In some embodiments, the term “sequentially” means within 7 days after the dose of the first component, e.g., within 4 days, 3 days, 2 days, or 1 day after the dose of the first component; and the term “simultaneously” means at the same time. In some embodiments, the term “sequentially” means within less than 1 day after the dose of the first component, e.g., within any of less than 24 hours, less than 20 hours, less than 15 hours, less than 10 hours, less than 12 hours, less than 8 hours, less than 6 hours, less than 3 hours, less than 2 hours, or less than 1 hour after the dose of the first component. In some embodiments, the anti-CD79b immunoconjugate and the anti-CD20 antibody are co-administered sequentially on about Day 1 of each 21-day cycle and the one or more chemotherapeutic agents are co-administered sequentially on about Day 2 of each 21-day cycle.

Anti-CD79b immunoconjugates, the anti-CD20 antibodies, and the one or more chemotherapeutic agents provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question.

The amount of co-administration of the anti-CD79b immunoconjugate, the anti-CD20 antibody, and the one or more chemotherapeutic agents, and the timing of co-administration will depend on the type (species, gender, age, weight, etc.) and condition of the patient being treated and the severity of the disease or condition being treated. The anti-CD79b immunoconjugate, the anti-CD20 antibody, and the one or more chemotherapeutic agents are suitably co-administered to the patient at one time or over a series of treatments, e.g., on the same day or on the day after.

In some embodiments, the dosage of anti-CD79b immunoconjugate (such as polatuzumab vedotin-piiq) is between about any of 1.4-5 mg/kg, 1.4-4 mg/kg, 1.4-3.2 mg/kg, 1.4-2.4 mg/kg, or 1.4-1.8 mg/kg. In some embodiments of any of the methods, the dosage of anti-CD79b immunoconjugate is about any of 1.4 mg/kg, 1.5 mg/kg. 1.6 mg/kg. 1.7 mg/kg, 1.8 mg/kg, 1.9 mg/kg, 2.0 mg/kg, 2.2 mg/kg, 2.4 mg/kg, 2.6 mg/kg, 2.8 mg/kg, 3.0 mg/kg, 3.2 mg/kg, 3.4 mg/kg, 3.6 mg/kg, 3.8 mg/kg, 4.0 mg/kg, 4.2 mg/kg, 4.4 mg/kg, 4.6 mg/kg, and/or 4.8 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments, the dosage of anti-CD79b immunoconjugate is about 2.4 mg/kg. In some embodiments, the dosage of anti-CD79b immunoconjugate is about 3.2 mg/kg. In some embodiments, the dosage of anti-CD79b immunoconjugate is about 3.6 mg/kg. In some embodiments of any of the methods, the anti-CD79b immunoconjugate is administered q3wk. In some embodiments of any of the methods, the anti-CD79b immunoconjugate is administered once in each 21-day cycle. In some embodiments of any of the methods, the anti-CD79b immunoconjugate is administered on about day 1 of each 21-day cycle. In some embodiments, the anti-CD79b immunoconjugate is administered via intravenous infusion. In some embodiments, the dosage administered via infusion is in the range of about 1 mg to about 2,000 mg per dose, generally every three weeks (e.g., on day 1 of each 21-day cycle) for a total of 1, 2, 3, 4, 5, 6, 7, 8, 9, 10, 11, 12, 13, 14, 15, 16, or more doses. In some embodiments, the dosage administered via infusion is in the range of about 1 mg to about 2,000 mg per dose, generally on about day 1 of each 21-day cycle for up to eight 21-day cycles. Alternatively, the dosage range is of about 1 mg to about 2,000 mg, about 1 mg to about 1,800 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose, generally on about day 1 of each 21-day cycle for a total of 1, 2, 3, 4, 5, 6, 7, 8, 9, 10, 11, 12, 13, 14, 15, 16, or more. In some embodiments, the dosage administered via infusion is in the range of about 1 μg/m² to about 10,000 μg/m² per dose, generally one dose every three weeks (e.g., on day 1 of each 21-day cycle) for up to eight 21-day cycles. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per week, multiple times per week, but less than once per day, multiple times per month but less than once per day, multiple times per month but less than once per week, once per month, once every three weeks, once every 21 days, once each 21-day cycle, on day 1 of each 21-day cycle, or intermittently to relieve or alleviate symptoms of the disease. Administration may continue at any of the disclosed intervals for up to eight 21-day cycles or until remission of the tumor or symptoms of the B-cell proliferative disorder (e.g., DLBCL) being treated. Administration may continue after remission or relief of symptoms is achieved where such remission or relief is prolonged by such continued administration.

In some embodiments, the dosage of the anti-CD20 antibody (e.g., rituximab) is between about 300-1600 mg/m² and/or 300-2000 mg. In some embodiments, the dosage of the anti-CD20 antibody is about any of 300 mg/m², 375 mg/m², 600 mg/m², 1000 mg/m², or 1250 mg/m² and/or 300 mg, 1000 mg, or 2000 mg. In some embodiments, the anti-CD20 antibody is rituximab and the dosage administered is 375 mg/m². In some embodiments, the anti-CD20 antibody is administered q3w (i.e., every 3 weeks). In some embodiments, the anti-CD20 antibody is administered once in each 21-day cycle (e.g., on day 1 of each 21-day cycle). In some embodiments, the anti-CD20 antibody is rituximab. In some embodiments, the dosage of rituximab may be 375 mg/m² on day 1 of each 21-day cycle. In some embodiments, the dosage of rituximab may be 375 mg/m² on day 1 of each 21-day cycle for up to eight 21-day cycles. In some embodiments, the dosage of rituximab may be 375 mg/m² on day 1 of each 21-day cycle for eight 21-day cycles.

In some embodiments, the anti-CD20 antibody (e.g., rituximab) may be administered once per week, multiple times per month but less than once per week, once per month, once every three weeks, once every 21 days, once each 21-day cycle, on day 1 of each 21-day cycle, or intermittently to relieve or alleviate symptoms of the disease. Administration may continue at any of the disclosed intervals for up to eight 21-day cycles or until remission of the tumor or symptoms of the B-cell proliferative disorder (e.g., DLBCL) being treated. Administration may continue after remission or relief of symptoms is achieved where such remission or relief is prolonged by such continued administration.

In some embodiments, the dosage of the one or more chemotherapeutic agents is between about 50 mg/m² to about 2000 mg/m². In some embodiments, the dosage of the one or more chemotherapeutic agents is between about 50 mg/m² to about 100 mg/m², about 100 mg/m² to about 200 mg/m², about 200 mg/m² to about 300 mg/m², about 300 mg/m² to about 400 mg/m², about 400 mg/m² to about 500 mg/m², about 500 mg/m² to about 600 mg/m², about 600 mg/m² to about 700 mg/m², about 700 mg/m² to about 800 mg/m², about 800 mg/m² to about 900 mg/m², about 900 mg/m² to about 1000 mg/m², about 1000 mg/m² to about 1100 mg/m², about 1100 mg/m² to about 1200 mg/m², about 1200 mg/m² to about 1300 mg/m², about 1300 mg/m² to about 1400 mg/m², about 1400 mg/m² to about 1500 mg/m², or about 1500 mg/m² to about 2000 mg/m².

In some embodiments, the dosage of the one or more chemotherapeutic agents comprises a dose of gemcitabine of about 500 mg/m² to about 1500 mg/m²(e.g., about 500 mg/m² to about 600 mg/m², about 600 mg/m² to about 700 mg/m², about 700 mg/m² to about 800 mg/m², about 800 mg/m² to about 900 mg/m², about 900 mg/m² to about 1000 mg/m², about 1000 mg/m² to about 1100 mg/m², about 1100 mg/m² to about 1200 mg/m², about 1200 mg/m² to about 1300 mg/m², about 1300 mg/m² to about 1400 mg/m², about 1400 mg/m² to about 1500 mg/m²). In some embodiments, the dosage of the one or more chemotherapeutic agents comprises a dose of gemcitabine of about 1000 mg/m². In some embodiments, the dose of gemcitabine is administered q3w or about once in each 21-day cycle (e.g., on day 2 of each 21 day cycle). In some embodiments, the dose of gemcitabine is administered once in each 21-day cycle (e.g., on day 2 of each 21-day cycle) for up to eight 21-day cycles. In some embodiments, the dose of gemcitabine is administered once in each 21-day cycle (e.g., on day 2 of each 21-day cycle) for eight 21-day cycles. In some embodiments, the dosage of the one or more chemotherapeutic agents comprises a dose of oxaliplatin of between about 50 mg/m² to about 200 mg/m²(e.g., 50 mg/m² to about 100 mg/m² or about 100 mg/m² to about 200 mg/m²). In some embodiments, the dosage of the one or more chemotherapeutic agents comprises a dose of oxaliplatin of about 100 mg/m². In some embodiments, the dose of oxaliplatin is administered q3w or about once in each 21-day cycle (e.g., on day 2 of each 21 day cycle). In some embodiments, the dose of oxaliplatin is administered once in each 21-day cycle (e.g., on day 2 of each 21-day cycle) for up to eight 21-day cycles. In some embodiments, the dose of gemcitabine is administered once in each 21-day cycle (e.g., on day 2 of each 21-day cycle) for eight 21-day cycles. In some embodiments, the one or more chemotherapeutic agents comprise gemcitabine and oxaliplatin, and the gemcitabine and the oxaliplatin are administered intravenously on about day 2 of each 21-day cycle. In some embodiments, the one or more chemotherapeutic agents comprise gemcitabine and oxaliplatin, and the gemcitabine and the oxaliplatin are administered intravenously on day 2 of each 21-day cycle for up to about eight cycles.

In some embodiments, the one or more chemotherapeutic agents (e.g., gemcitabine and oxaliplatin) are administered once per week, multiple times per week, but less than once per day, multiple times per month but less than once per day, multiple times per month but less than once per week, once per month, once every three weeks, once in each 21-day cycle, on about day 2 of each 21-day cycle, or intermittently to relieve or alleviate symptoms of the disease. Administration may continue at any of the disclosed intervals for up to about eight 21-day cycles or until remission of the tumor or symptoms of the B-cell proliferative disorder being treated. Administration may continue after remission or relief of symptoms is achieved where such remission or relief is prolonged by such continued administration.

Exemplary dosing regimens for the combination therapy of anti-CD79b immunoconjugates (such as polatuzumab vedotin-piiq) and other agents include, but are not limited to, anti-CD79b immunoconjugate (such as huMA79bv28-MC-vc-PAB-MMAE) administered at about 1.4-5 mg/kg on about day 1 of each 21 day cycle, plus an anti-CD20 antibody (e.g., rituximab) administered at about 300-1600 mg/m² on about day 1 of each 21 day cycle, plus one or more chemotherapeutic agents (e.g., gemcitabine and oxaliplatin) administered at about 50 mg/m² to about 2000 mg/m² (e.g., gemcitabine administered at about 500 mg/m² to about 2000 mg/m² and oxaliplatin administered at about 50 mg/m² to about 200 mg/m²) on about day 2 of each 21 day cycle. In some embodiments, the anti-CD79b immunoconjugate (such as polatuzumab vedotin-piiq) is administered at about 1.8 mg/kg on about day 1 of each 21 day cycle, the anti-CD20 antibody (e.g., rituximab) is administered at about 375 mg/m² on about day 1 of each 21 day cycle, and the one or more chemotherapeutic agents (e.g., gemcitabine administered at about 1000 mg/m² and oxaliplatin administered at about 100 mg/m²) are administered on about day 2 of each 21 day cycle. In some embodiments, the anti-CD79b immunoconjugate is administered at about 1.8 mg/kg. In some embodiments, the anti-CD79b immunoconjugate is polatuzumab vedotin-piiq and is administered at about 1.8 mg/kg. In some embodiments, the anti-CD20 antibody is administered at about 375 mg/m² In some embodiments, the anti-CD20 antibody is rituximab and is administered at about 375 mg/m². In some embodiments, the one or more chemotherapeutic agents comprise gemcitabine administered at about 1000 mg/m² and oxaliplatin administered at about 100 mg/m². In some embodiments, the anti-CD79b immunoconjugate is polatuzumab vedotin-piiq and is administered at about 1.8 mg/kg, the anti-CD20 antibody is rituximab and is administered at about 375 mg/m² and the one or more chemotherapeutic agents are gemcitabine administered at about 1000 mg/m² and oxaliplatin administered at about 100 mg/m².

An immunoconjugate provided herein, an anti-CD20 antibody provided herein, and one or more chemotherapeutic agents provided herein for use in any of the therapeutic methods described herein can be administered by any suitable means, including parenteral, intrapulmonary, and intranasal, and, if desired for local treatment, intralesional administration. Parenteral infusions include intramuscular, intravenous (e.g., intravenous infusion), intraarterial, intraperitoneal, or subcutaneous administration. Dosing can be by any suitable route, e.g., by injections, such as intravenous or subcutaneous injections, depending in part on whether the administration is brief or chronic. Various dosing schedules, including, but not limited to single or multiple administrations over various time-points, bolus administration, and pulse infusion are contemplated herein.

Provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

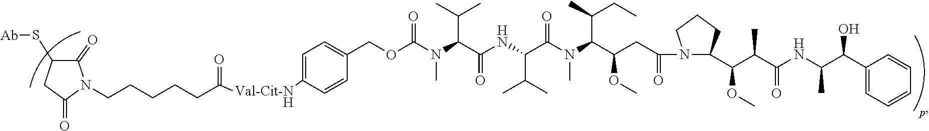

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8; (b) an anti-CD20 antibody, and (c) one or more chemotherapeutic agents. In some embodiments, the immunoconjugate comprises an anti-CD79b antibody that comprises a heavy chain variable domain (VH) comprising the amino acid sequence of SEQ ID NO: 19 and a light chain variable domain (VL) comprising the amino acid sequence of SEQ ID NO: 20.

Also provided herein are methods for treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

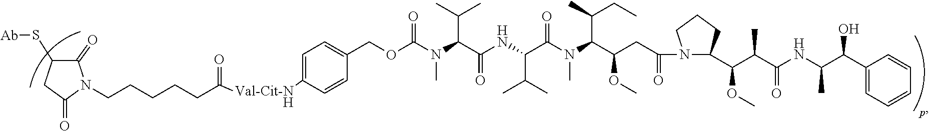

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein, after administration of the immunoconjugate, the rituximab, the gemcitabine, and the oxaliplatin, the human does not experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods for treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

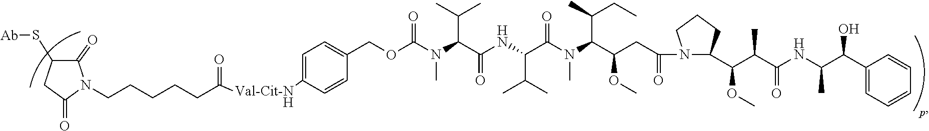

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein administration of the immunoconjugate, the rituximab, the gemcitabine, and the oxaliplatin does not result in peripheral neuropathy of Grade 3 or greater (e.g., any of Grade 3 or greater, Grade 4 or greater, or Grade 5 or greater) in the human that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods for treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

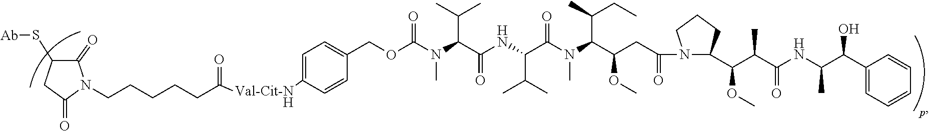

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein, among a plurality of humans treated, after administration of the immunoconjugate, the rituximab, the gemcitabine, and the oxaliplatin, 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, among a plurality of humans treated, after administration of the immunoconjugate, the rituximab, the gemcitabine, and the oxaliplatin, fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experience peripheral neuropathy of Grade 3 (e.g., any of Grade 3 or greater, Grade 4 or greater, or Grade 5 or greater) or greater that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods for treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

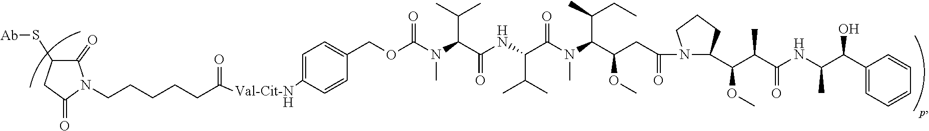

wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) gemcitabine, and (d) oxaliplatin; wherein administration of the immunoconjugate, the rituximab, the gemcitabine, and the oxaliplatin to a plurality of humans results in 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, administration of the immunoconjugate, the rituximab, the gemcitabine, and the oxaliplatin to a plurality of humans results in fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 35. In some embodiments, p is between 2 and 7, between 2 and 6, between 2 and 5, between 3 and 5, or between 3 and 4. In some embodiments, p is between 2 and 5. In some embodiments, p is between 3 and 4. In some embodiments, p is 3.5. In some embodiments, the anti-CD79b immunoconjugate is polatuzumab vedotin-piiq. In some embodiments, the anti-CD20 antibody is rituximab. In some embodiments, the one or more chemotherapeutic agents comprise any of the chemotherapeutic agents provided herein. In some embodiments, the one or more chemotherapeutic agents comprise gemcitabine. In some embodiments, the one or more chemotherapeutic agents comprise oxaliplatin. In some embodiments, the one or more chemotherapeutic agents are gemcitabine and oxaliplatin.

In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin. In certain embodiments, p is between 2 and 5. In certain embodiments, p is 2. In some embodiments, the immunoconjugate is administered at a dose that is from about 1 mg/kg to about 5 mg/kg. In some embodiments, the immunoconjugate is administered at a dose of about 1.2 mg/kg, about 1.8 mg/kg, about 2.4 mg/kg, about 3.6 mg/kg, or about 4.8 mg/kg. In some embodiments, the immunoconjugate is administered at a dose of about 1.8 mg/kg.

As used herein, the term “iladatuzumab vedotin” refers to an anti-CD79b immunoconjugate having the International Nonproprietary Names for Pharmaceutical Substances (INN) Number 10647, or the CAS Registry Number 1906205-77-3. Iladatuzumab vedotin is also interchangeably referred to as “DCDS0780A” or “RO7032005”.

The anti-CD79b immunoconjugate (e.g., polatuzumab vedotin-piiq), the anti-CD20 antibody (such as rituximab), and the one or more chemotherapeutic agents (e.g., gemcitabine and oxaliplatin) may be administered by the same route of administration or by different routes of administration. In some embodiments, the anti-CD79b immunoconjugate is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (such as rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the one or more chemotherapeutic agents (e.g., gemcitabine and oxaliplatin) are administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD79b immunoconjugate, the anti-CD20 antibody (such as rituximab), and the one or more chemotherapeutic agents (e.g., gemcitabine and oxaliplatin) are each administered via intravenous infusion. An effective amount of the anti-CD79b immunoconjugate (e.g., polatuzumab vedotin-piiq), the anti-CD20 antibody (such as rituximab), and the one or more chemotherapeutic agents (e.g., gemcitabine and oxaliplatin) may be administered for the prevention or treatment of disease.

In some embodiments, the anti-CD79b immunoconjugate (e.g., polatuzumab vedotin-piiq) is administered at a dose between about 1.4 mg/kg to about 2 mg/kg (e.g., about 1.4 mg/kg to about 1.6 mg/kg, about 1.6 mg/kg to about 1.8 mg/kg, or about 1.8 mg/kg to about 2 mg/kg). In some embodiments, the anti-CD79b immunoconjugate is administered at a dose of 1.8 mg/kg. In some embodiments, the anti-CD79b immunoconjugate is polatuzumab vedotin-piiq. In some embodiments, the polatuzumab vedotin-piiq is administered at a dose of 1.8 mg/kg. Alternatively or additionally, in some embodiments, the anti-CD20 antibody (e.g., rituximab) is administered at a dose of between about 300-1800 mg/m² (e.g., about 300 mg/m² to about 600 mg/m², about 600 mg/m² to about 900 mg/m², about 900 mg/m² to about 1200 mg/m², about 1200 mg/m² to about 1500 mg/m², or about 1500 mg/m² to about 1800 mg/m²) and/or about 300-2000 mg (e.g., about 300 mg to about 600 mg, about 600 mg to about 900 mg, about 900 mg to about 1200 mg, about 1200 mg to about 1500 mg, about 1500 mg to about 1750 mg, or about 1750 mg to about 2000 mg). In some embodiments, the anti-CD20 antibody is rituximab. In some embodiments, the rituximab is administered at a dose of about 375 mg/m². Alternatively, or additionally, in some embodiments, the one or more chemotherapeutic agents (e.g., gemcitabine and oxaliplatin) are administered at a dose of between at about 50 mg/m² to about 2000 mg/m²(e.g., about 50 mg/m² to about 100 mg/m², about 100 mg/m² to about 200 mg/m², about 200 mg/m² to about 400 mg/m², about 400 mg/m² to about 600 mg/m², about 600 mg/m² to about 800 mg/m², about 800 mg/m² to about 1000 mg/m², about 1000 mg/m² to about 1200 mg/m², about 1200 mg/m² to about 1400 mg/m², about 1400 mg/m² to about 1600 mg/m², about 1600 mg/m² to about 1800 mg/m², or about 1800 mg/m² to about 2000 mg/m²). In some embodiments, the one or more chemotherapeutic agents comprise gemcitabine. In some embodiments, gemcitabine is administered at a dose of about 1000 mg/m². In some embodiments, the one or more chemotherapeutic agents comprise oxaliplatin. In some embodiments, the oxaliplatin is administered at a dose of about 100 mg/m². In some embodiments, the one or more chemotherapeutic agents are gemcitabine and oxaliplatin, and the gemcitabine is administered at a dose of about 1000 mg/m² and the oxaliplatin is administered at a dose of about 100 mg/m².

In some embodiments, the anti-CD20 antibody is rituximab. In some embodiments, the immunoconjugate (e.g., polatuzumab vedotin-piiq) is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m². In some embodiments, the polatuzumab vedotin-piiq is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m².

In some embodiments, the anti-CD79b immunoconjugate (e.g., polatuzumab vedotin-piiq), the anti-CD20 antibody (e.g., rituximab), and the one or more chemotherapeutic agents (e.g., gemcitabine and oxaliplatin) are administered for at least one 21-day cycle (e.g., any of about one, about two, about three, about four, about five, about six, about seven, or about eight, or more 21-day cycles).

In some embodiments, the anti-CD79b immunoconjugate (e.g., polatuzumab vedotin-piiq), the anti-CD20 antibody (e.g., rituximab), and the one or more chemotherapeutic agents (e.g., gemcitabine and oxaliplatin) are administered for up to eight 21-day cycles (e.g., any of about one, about two, about three, about four, about five, about six, about seven, or about eight 21-day cycles). In some embodiments, the immunoconjugate (e.g., polatuzumab vedotin-piiq) is administered intravenously on about Day 1 of each 21-day cycle. In some embodiments, the immunoconjugate is polatuzumab vedotin-piiq and the polatuzumab vedotin-piiq is administered intravenously on about Day 1 of each 21-day cycle. Alternatively or additionally, in some embodiments, the anti-CD20 antibody (e.g., rituximab) is administered intravenously on about Day 1 of each 21-day cycle. In some embodiments, the anti-CD20 antibody is rituximab, and the rituximab is administered on about Day 1 of each 21-day cycle. Alternatively or additionally, in some embodiments, the one or more chemotherapeutic agents (e.g., gemcitabine and oxaliplatin) are administered intravenously on about Day 2 of each 21-day cycle. In some embodiments, the chemotherapeutic agents are gemcitabine and oxaliplatin and the gemcitabine and oxaliplatin are administered intravenously on about Day 2 of each 21-day cycle.

In some embodiments, the immunoconjugate (e.g., polatuzumab vedotin-piiq), the anti-CD20 antibody (e.g., rituximab), and the one or more chemotherapeutic agents (e.g., gemcitabine and oxaliplatin) are administered for at least one 21-day cycle, wherein the immunoconjugate and the anti-CD20 antibody are administered intravenously on about Day 1 of each 21-day cycle, and wherein the one or more chemotherapeutic agents are administered intravenously on about Day 2 of each 21-day cycle. In some embodiments, the immunoconjugate (e.g., polatuzumab vedotin-piiq), the anti-CD20 antibody (e.g., rituximab), and the one or more chemotherapeutic agents (e.g., gemcitabine and oxaliplatin) are administered for up to eight 21-day cycles, wherein the immunoconjugate and the anti-CD20 antibody are administered intravenously on about Day 1 of each 21-day cycle, and wherein the one or more chemotherapeutic agents are administered intravenously on about Day 2 of each 21-day cycle. In some embodiments, the one or more chemotherapeutic agents are gemcitabine and oxaliplatin. In some embodiments, the anti-CD20 antibody is rituximab. In some embodiments, the immunoconjugate (e.g., polatuzumab vedotin-piiq), the rituximab, the gemcitabine, and the oxaliplatin are administered for up to eight 21-day cycles, wherein the immunoconjugate and the rituximab are administered intravenously on about Day 1 of each 21-day cycle, and wherein the gemcitabine and the oxaliplatin are administered intravenously on about Day 2 of each 21-day cycle. In some embodiments, the immunoconjugate (e.g., polatuzumab vedotin-piiq) is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m², wherein the immunoconjugate, the rituximab, the gemcitabine, and the oxaliplatin are administered for up to eight 21-day cycles, and wherein the immunoconjugate and the rituximab are administered on Day 1 of each 21-day cycle and the gemcitabine and the oxaliplatin are administered on Day 2 of each 21-day cycle. In some embodiments, the immunoconjugate (e.g., polatuzumab vedotin-piiq), the rituximab, the gemcitabine, and the oxaliplatin are administered for at least one 21-day cycle, wherein the immunoconjugate and the rituximab are administered intravenously on about Day 1 of each 21-day cycle, and wherein the gemcitabine and the oxaliplatin are administered intravenously on about Day 2 of each 21-day cycle. In some embodiments, the immunoconjugate (e.g., polatuzumab vedotin-piiq) is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m², wherein the immunoconjugate, the rituximab, the gemcitabine, and the oxaliplatin are administered for at least one 21-day cycles, and wherein the immunoconjugate and the rituximab are administered on Day 1 of each 21-day cycle and the gemcitabine and the oxaliplatin are administered on Day 2 of each 21-day cycle.

Provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

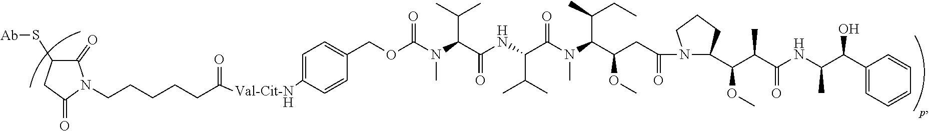

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5; (b) rituximab; (c) gemcitabine; and (d) oxaliplatin; wherein the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate, the rituximab, the gemcitabine, and the oxaliplatin are administered for up to eight 21-day cycles, wherein the immunoconjugate and the rituximab are administered intravenously on Day 1 of each 21-day cycle, and wherein the gemcitabine and the oxaliplatin are administered intravenously on Day 2 of each 21-day cycle. In some embodiments, the immunoconjugate is polatuzumab vedotin-piiq.

Provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

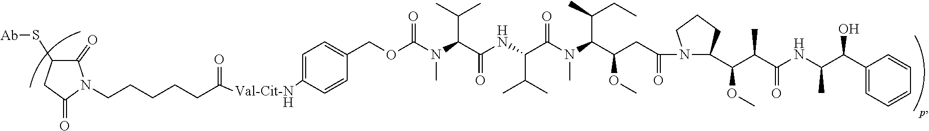

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5; (b) rituximab; (c) gemcitabine; and (d) oxaliplatin; wherein the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and wherein the immunoconjugate, the rituximab, the gemcitabine, and the oxaliplatin are administered for at least one 21-day cycle, wherein the immunoconjugate and the rituximab are administered intravenously on Day 1 of each 21-day cycle, and wherein the gemcitabine and the oxaliplatin are administered intravenously on Day 2 of each 21-day cycle. In some embodiments, the immunoconjugate is polatuzumab vedotin-piiq.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

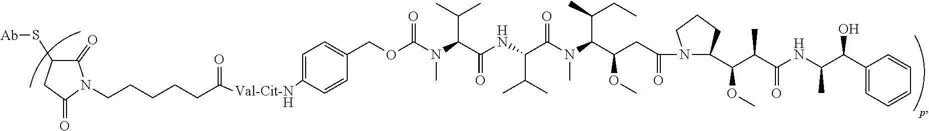

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, after administration of the immunoconjugate, the rituximab, the gemcitabine, and the oxaliplatin, the human does not experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

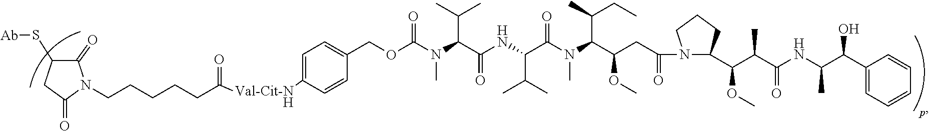

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, after administration of the immunoconjugate, the rituximab, the gemcitabine, and the oxaliplatin, the human does not experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

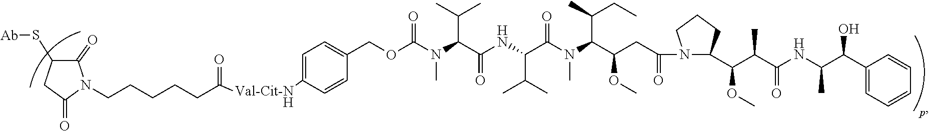

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin does not result in peripheral neuropathy of Grade 3 or greater (e.g., any of Grade 3 or greater, Grade 4 or greater, or Grade 5 or greater) in the human that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

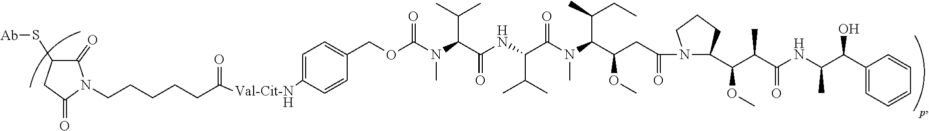

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin does not result in peripheral neuropathy of Grade 3 or greater (e.g., any of Grade 3 or greater, Grade 4 or greater, or Grade 5 or greater) in the human that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

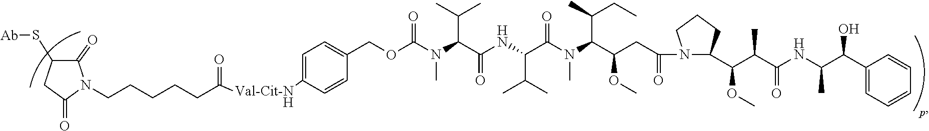

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein, among a plurality of humans treated, after administration of the immunoconjugate, the rituximab, the gemcitabine, and the oxaliplatin, 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles. In some embodiments, among a plurality of humans treated, after administration of the immunoconjugate, the rituximab, the gemcitabine, and the oxaliplatin, fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experience peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

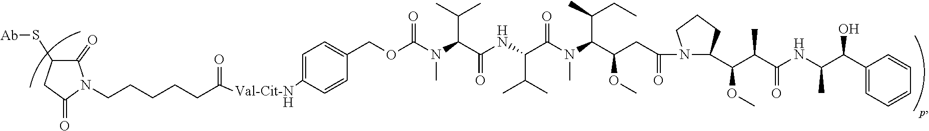

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for up to eight 21-day cycles, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin to a plurality of humans results in 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles. In some embodiments, administration of the immunoconjugate, the rituximab, the gemcitabine, and the oxaliplatin to a plurality of humans results in fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula

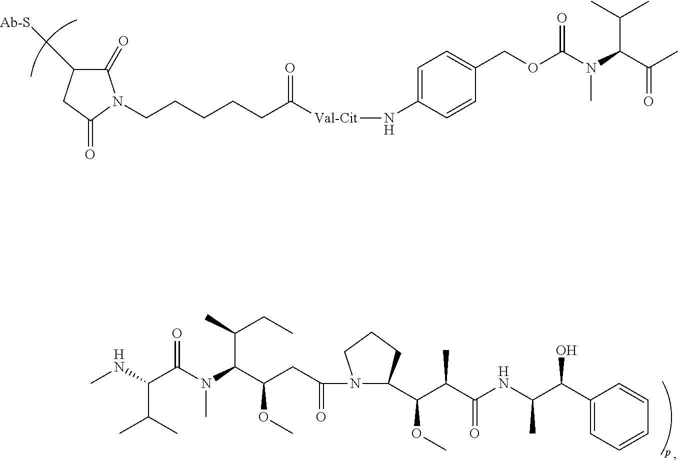

wherein Ab is an anti-CD79b antibody comprising (i) a heavy chain comprising the amino acid sequence of SEQ ID NO: 36 and (ii) a light chain comprising the amino acid sequence of SEQ ID NO: 35, and wherein p is between 2 and 5, (b) rituximab, (c) gemcitabine, and (d) oxaliplatin; wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; wherein the immunoconjugate and the rituximab are administered intravenously on Day 1 of each 21-day cycle, and wherein the gemcitabine and the oxaliplatin are administered intravenously on Day 2 of each 21-day cycle; and wherein administration of the immunoconjugate, the rituximab, the gemcitabine, and the oxaliplatin to a plurality of humans results in 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles. In some embodiments, administration of the immunoconjugate, the rituximab, the gemcitabine, and the oxaliplatin to a plurality of humans results in fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, the immunoconjugate, the rituximab, the gemcitabine, and the oxaliplatin have been administered for at least four 21-day cycles.

In some embodiments, the anti-CD20 antibody (e.g., rituximab) is administered before (e.g., any of up to about 1 hour, up to about 2 hours, up to about 4 hours, up to about 6 hours, up to about 12 hours, up to about 16 hours, up to about 18 hours, up to about 22 hours, up to about 24 hours, or up to about 1 day, up to about 2 days, up to about 3 days, or more before) the immunoconjugate (e.g., polatuzumab vedotin-piiq). In some embodiments, the anti-CD20 antibody is rituximab, and the rituximab is administered before the immunoconjugate. In some embodiments, the immunoconjugate is administered before (e.g., any of up to about 1 hour, up to about 2 hours, up to about 4 hours, up to about 6 hours, up to about 12 hours, up to about 16 hours, up to about 18 hours, up to about 22 hours, up to about 24 hours, or up to about 1 day, up to about 2 days, up to about 3 days, or more before) the anti-CD20 antibody. In some embodiments, the anti-CD20 antibody is rituximab, and the immunoconjugate is administered before the rituximab.

In some embodiments, the one or more chemotherapeutic agents are administered sequentially. In some embodiments, the chemotherapeutic agents are gemcitabine and oxaliplatin. In some embodiments, the gemcitabine is administered before (e.g., any of up to about 1 hour, up to about 2 hours, up to about 4 hours, up to about 6 hours, up to about 12 hours, up to about 16 hours, up to about 18 hours, up to about 22 hours, up to about 24 hours, or up to about 1 day, up to about 2 days, up to about 3 days, or more before) the oxaliplatin. In some embodiments, the oxaliplatin is administered before (e.g., any of up to about 1 hour, up to about 2 hours, up to about 4 hours, up to about 6 hours, up to about 12 hours, up to about 16 hours, up to about 18 hours, up to about 22 hours, up to about 24 hours, or up to about 1 day, up to about 2 days, up to about 3 days, or more before) the gemcitabine.

In some embodiments, the immunoconjugate, the anti-CD20 antibody, and the one or more chemotherapeutic agents are administered for any of one, two, three, four, five, six, seven, or eight 21-day cycles. In some embodiments, the chemotherapeutic agents are gemcitabine and oxaliplatin. In some embodiments, the anti-CD20 antibody is rituximab. In some embodiments, the immunoconjugate, the rituximab, the gemcitabine, and the oxaliplatin are administered for any of one, two, three, four, five, six, seven, or eight 21-day cycles. In some embodiments, the immunoconjugate, the rituximab, the gemcitabine, and the oxaliplatin are administered for eight 21-day cycles.

In some embodiments, which may be combined with any of the preceding embodiments, the immunoconjugate, the rituximab, the gemcitabine, and the oxaliplatin are administered for at least two, at least three, at least four, at least five, at least six, or at least seven 21-day cycles. In some embodiments, the immunoconjugate, the rituximab, the gemcitabine, and the oxaliplatin are administered for eight 21-day cycles. In some embodiments, the rituximab is administered before the immunoconjugate. In some embodiments, the gemcitabine is administered before the oxaliplatin. In some embodiments, the immunoconjugate is polatuzumab vedotin-piiq.

In some embodiments, the human has received at least one prior therapy for DLBCL. In some embodiments, the human is an adult. In some embodiments, the human has histologically confirmed diffuse large B-cell lymphoma, not otherwise specified (NOS), or the human has a history of transformation of indolent disease to DLBCL. In some embodiments, the human has received at least one prior systemic therapy for DLBCL. In some embodiments, the human has received at least two prior therapies for DLBCL. For example, in some cases, the human has received prior autologous hematopoietic stem cell transplantation (HSCT) (chemotherapy followed by consolidative autologous HSCT is counted as one line of prior therapy). In another example, the human has received a prior allogeneic HSCT and the human is no longer receiving immunosuppressive therapy and has no active graft versus host disease (GVHD) (chemotherapy followed by allogeneic HSCT is counted as one line of prior therapy). In some embodiments, the human received prior local therapies, e.g., radiotherapy. In some embodiments, the DLBCL is relapsed or refractory. In some embodiments, the DLBCL is relapsed if it recurs following a response that lasted ≥6 months from completion of the last line of therapy. In some embodiments, the DLBCL is refractory if it progresses during prior therapy or progresses within 6 months (<6 months) of prior therapy. In some embodiments, the human has at least one bi-dimensionally measurable lesion, e.g., a lesion that is greater than 1.5 cm in its longest dimension as measured by computed tomography (CT) or magnetic resonance imaging (MRI). In some embodiments, the human has an Eastern Cooperative Oncology Group (ECOG) performance status of 0, 1, or 2. In some embodiments, the human does not have a planned autologous or allogeneic stem cell transplantation (SCT). In some embodiments, the human has not had prior therapy with a combination of gemcitabine and a platinum-based agent. In some embodiments, the human does not have peripheral neuropathy of greater than Grade 1 according to National Cancer Institute Common Terminology Criteria for Adverse Events, Version 5.0. In some embodiments, the human does not have primary or secondary central nervous system (CNS) lymphoma. In some embodiments, the human does not have Richter's transformation or prior capillary leak syndrome (CLL). In some embodiments, the human is not a candidate for hematopoietic stem cell transplantation (HSCT) prior to administration of the immunoconjugate, the rituximab, the gemcitabine, and the oxaliplatin. In some embodiments, the human is not a candidate for autologous hematopoietic stem cell transplantation (HSCT) prior to administration of the immunoconjugate, the rituximab, the gemcitabine, and the oxaliplatin. In some embodiments, the human has received at least two prior therapies for DLBCL. In some embodiments, the human has not received a prior therapy with polatuzumab vedotin-piiq for DLBCL. In some embodiments, the human is an adult. In some embodiments, the human adult has relapsed or refractory diffuse large B-cell lymphoma, not otherwise specified.

In some embodiments, diffuse large B-cell lymphoma (DLBCL), not otherwise specified (NOS) (e.g., relapsed or refractory diffuse large B-cell lymphoma, not otherwise specified) refers to DLBCL that does not fit the distinctive clinical presentation, tissue morphology, neoplastic cell phenotype, and/or pathogen-associated criteria of other DLBCL subtypes. DLBC NOS is generally an aggressive disease representing about 80-85% of all DLBCL cases. DLBCL NOS pateints reated with standard chemotherapy regimens have an overall long-term survival rate of about 65%. See, e.g., Grimm et al (2019) Annals of Diagnostic Pathology, 38:6-10.

In some embodiments, administration of the immunoconjugate, the rituximab, the gemcitabine, and the oxaliplatin does not result in peripheral neuropathy of Grade 3 or greater (e.g., any of Grade 3 or greater, Grade 4 or greater, or Grade 5 or greater) in the human that does not resolve to Grade 1 or lower within 14 days. In some embodiments, administration of the immunoconjugate, the rituximab, the gemcitabine, and the oxaliplatin does not result in peripheral neuropathy of Grade 4 or greater (e.g., any of Grade 4 or greater, or Grade 5 or greater) in the human. In some embodiments, administration of the immunoconjugate, the rituximab, the gemcitabine, and the oxaliplatin to a plurality of humans results in 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, administration of the immunoconjugate, the rituximab, the gemcitabine, and the oxaliplatin to a plurality of humans results in fewer than about 40% (e.g., any of fewer than about 40%, about 39% or fewer, about 38% or fewer, about 37% or fewer, about 36% or fewer, about 35% or fewer, about 34% or fewer, about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In certain embodiments, the immunoconjugate, the rituximab, the gemcitabine, and the oxaliplatin have been administered for at least four 21-day cycles.

In some embodiments, administration of the immunoconjugate, the rituximab, the gemcitabine, and the oxaliplatin to a plurality of humans results in less than about 8% (e.g., less than any of about 8%, 7%, 6%, 5%, 4%, 3%, 2%, or 1%) of the humans in the plurality experiencing peripheral neuropathy of Grade 3 or greater (e.g., any of Grade 3 or greater, Grade 4 or greater, or Grade 5 or greater). In some embodiments, administration of the immunoconjugate, the rituximab, the gemcitabine, and the oxaliplatin to a plurality of humans results in less than about 6% (e.g., less than any of about 6%, 5%, 4%, 3%, 2%, or 1%) of the humans in the plurality experiencing peripheral neuropathy that results in discontinuation of treatment with the immunoconjugate, the rituximab, the gemcitabine, and the oxaliplatin. In some embodiments, the immunoconjugate, the rituximab, the gemcitabine, and the oxaliplatin have been administered for at least four 21-day cycles.

In some embodiments, after administration of the immunoconjugate, the rituximab, the gemcitabine, and the oxaliplatin, the human does not experience peripheral neuropathy of Grade 4 or greater (e.g., any of Grade 4 or greater, or Grade 5 or greater). In some embodiments, administration of the immunoconjugate, the rituximab, the gemcitabine, and the oxaliplatin does not result peripheral neuropathy of Grade 4 or greater (e.g., any of Grade 4 or greater, or Grade 5 or greater) in the human. In some embodiments, after administration of the immunoconjugate, the rituximab, the gemcitabine, and the oxaliplatin, the human does not experience neurotoxicity of Grade 4 or greater (e.g., any of Grade 4 or greater, or Grade 5 or greater). In some embodiments, administration of the immunoconjugate, the rituximab, the gemcitabine, and the oxaliplatin does not result in neurotoxicity of Grade 4 or greater (e.g., any of Grade 4 or greater, or Grade 5 or greater) in the human. In some embodiments, neurotoxicity refers to sensory and/or motor peripheral neuropathy. In certain embodiments, the immunoconjugate, the rituximab, the gemcitabine, and the oxaliplatin have been administered for at least four 21-day cycles.

In some embodiments, administration of the immunoconjugate, the rituximab, the gemcitabine, and the oxaliplatin results in survival of the human for at least about 10 months or more, at least about 11 months or more, at least about 12 months or more, at least about 13 months or more, at least about 14 months or more, or at least about 15 months or more after the start of treatment with the immunoconjugate, the rituximab, the gemcitabine, and the oxaliplatin. In some embodiments, administration of the immunoconjugate, the rituximab, the gemcitabine, and the oxaliplatin to a plurality of humans results in a median overall survival of the humans in the plurality of at least about 10 months or more, at least about 11 months or more, at least about 12 months or more, at least about 13 months or more, at least about 14 months or more, or at least about 15 months or more after the start of treatment with the immunoconjugate, the rituximab, the gemcitabine, and the oxaliplatin. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in overall survival (OS) time, e.g., as compared to administration of an anti-CD20 antibody (e.g., rituximab) and one or more chemotherapeutic agents (e.g., gemcitabine and oxaliplatin). OS is measured from the first administration of the immunoconjugate, the anti-CD20 antibody, and the one or more chemotherapeutic agents to the time of an occurrence of death from any cause. In some embodiments, OS, e.g., median OS, is measured in days, weeks, months, or years. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in OS, e.g., median OS, of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). OS, e.g., the median OS, can be estimated according to any method known in the art. In some cases, OS, e.g., the median OS, is estimated using the Kaplan-Meier methodology. In some embodiments, estimates of treatment effect are expressed as hazard ratios for death using a stratified Cox proportional-hazards analysis (e.g., including 95% confidence limits). In some embodiments, the Brookmeyer-Crowley methodology is used to construct the 95% confidence interval for the median OS. In some embodiments, administration of the immunoconjugate, the rituximab, the gemcitabine, and the oxaliplatin results in an increase in survival of the human, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in overall survival of the humans in the plurality, e.g., median OS, of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, which may be combined with any of the preceding embodiments, administration of the immunoconjugate, the rituximab, the gemcitabine, and the oxaliplatin to a plurality of humans results in a 1-year overall survival rate of at least about 42% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 2-year overall survival rate of at least about 67% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 3.5-year overall survival rate of at least about 38% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 5-year overall survival rate of at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, in the 1-year, 2-year, 3.5-year, or 5-year overall survival rate compared to a corresponding plurality of humans administered rituximab, gemcitabine, and oxaliplatin. In some embodiments, a 1-year overall survival rate refers to the proportion of humans in a plurality of humans administered the immunoconjugate, the rituximab, the gemcitabine, and the oxaliplatin that do not have an occurrence of death from any cause at 1 year after the start of treatment with the immunoconjugate, the rituximab, the gemcitabine, and the oxaliplatin. In some embodiments, a 2-year overall survival rate refers to the proportion of humans in a plurality of humans administered the immunoconjugate, the rituximab, the gemcitabine, and the oxaliplatin that do not have an occurrence of death from any cause at 2 years after the start of treatment with the immunoconjugate, the rituximab, the gemcitabine, and the oxaliplatin. In some embodiments, a 3.5-year overall survival rate refers to the proportion of humans in a plurality of humans administered the immunoconjugate, the rituximab, the gemcitabine, and the oxaliplatin that do not have an occurrence of death from any cause at 3.5 years after the start of treatment with the immunoconjugate, the rituximab, the gemcitabine, and the oxaliplatin. In some embodiments, a 5-year overall survival rate refers to the proportion of humans in a plurality of humans administered the immunoconjugate, the rituximab, the gemcitabine, and the oxaliplatin that do not have an occurrence of death from any cause at 5 years after the start of treatment with the immunoconjugate, the rituximab, the gemcitabine, and the oxaliplatin.

In some embodiments, administration of the immunoconjugate, the rituximab, the gemcitabine, and the oxaliplatin results in progression-free survival of the human. In some embodiments, administration of the immunoconjugate, the rituximab, the gemcitabine, and the oxaliplatin results in progression-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 4 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 5 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 6 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 9.5 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 11 months after the start of treatment with the immunoconjugate, the rituximab, the gemcitabine, and the oxaliplatin. In some embodiments, administration of the immunoconjugate, the rituximab, the gemcitabine, and the oxaliplatin results in progression-free survival of the human for at least about 14 months after the start of treatment with the immunoconjugate, the rituximab, the gemcitabine, and the oxaliplatin. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the time of progression free survival (PFS), e.g., as compared to administration of an anti-CD20 antibody (e.g., rituximab) and one or more chemotherapeutic agents (e.g., gemcitabine and oxaliplatin). In some embodiments, PFS is measured from the first administration of the immunoconjugate (e.g., polatuzumab vedotin-piiq), the anti-CD20 antibody (e.g., rituximab), and the one or more chemotherapeutic agents (e.g., gemcitabine and oxaliplatin) to the time of a first occurrence of disease progression according to the Lugano 2014 response criteria (Cheson et al., (2014) J Clin Oncol 32:3059-3068), or death from any cause. In some embodiments, PFS is measured in days, weeks, months, or years. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in PFS of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, PFS is calculated using the Kaplan-Meier methodology. In some embodiments, estimates of treatment effect are expressed as hazard ratios using a stratified Cox proportional-hazards analysis (e.g., including 95% confidence limits). In some embodiments, administration of the immunoconjugate, the rituximab, the gemcitabine, and the oxaliplatin results in an increase in progression-free survival, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in progression-free survival (e.g., median progression-free survival) of the humans in the plurality, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to the progression-free survival (e.g., median progression-free survival) of a corresponding plurality of humans administered rituximab, gemcitabine, and oxaliplatin. In some embodiments, progression-free survival is measured from the first administration of the immunoconjugate, the rituximab, the gemcitabine, and the oxaliplatin to the first occurrence of disease progression (based on response including PET CT data or not including any PET data) according to Lugano 2014 response criteria, or death from any cause. In some embodiments, administration of the immunoconjugate, the rituximab, the gemcitabine, and the oxaliplatin to a plurality of humans results in a 1-year progression-free survival rate of at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 2-year progression-free survival rate of at least about 63% or greater, at least about 65% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in a 5-year progression-free survival rate of at least about 14% or greater, at least about 15% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, in the 1-year, 2-year, 3.5-year, or 5-year progression-free survival rate compared to a corresponding plurality of humans administered rituximab, gemcitabine, and oxaliplatin. In some embodiments, a 1-year progression-free survival rate refers to the proportion of humans in a plurality of humans administered the immunoconjugate, the rituximab, the gemcitabine, and the oxaliplatin that do not have an occurrence of disease progression (based on response including PET CT data or not including any PET data) according to Lugano 2014 response criteria, or death from any cause, at 1 year after the start of treatment with the immunoconjugate, the rituximab, the gemcitabine, and the oxaliplatin. In some embodiments, a 2-year progression-free survival rate refers to the proportion of humans in a plurality of humans administered the immunoconjugate, the rituximab, the gemcitabine, and the oxaliplatin that do not have an occurrence of disease progression (based on response including PET CT data or not including any PET data) according to Lugano 2014 response criteria, or death from any cause, at 2 years after the start of treatment with the immunoconjugate, the rituximab, the gemcitabine, and the oxaliplatin. In some embodiments, a 3.5-year progression-free survival rate refers to the proportion of humans in a plurality of humans administered the immunoconjugate, the rituximab, the gemcitabine, and the oxaliplatin that do not have an occurrence of disease progression (based on response including PET CT data or not including any PET data) according to Lugano 2014 response criteria, or death from any cause, at 3.5 years after the start of treatment with the immunoconjugate, the rituximab, the gemcitabine, and the oxaliplatin. In some embodiments, a 5-year progression-free survival rate refers to the proportion of humans in a plurality of humans administered the immunoconjugate, the rituximab, the gemcitabine, and the oxaliplatin that do not have an occurrence of disease progression (based on response including PET CT data or not including any PET data) according to Lugano 2014 response criteria, or death from any cause, at 5 years after the start of treatment with the immunoconjugate, the rituximab, the gemcitabine, and the oxaliplatin.

In some embodiments, OS and PFS are assessed in patients who undergo HSCT.

In some embodiments, the response to administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein is measured by any method known in the art. In some embodiments, the response to administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein is measured by positron emission tomography (PET) and/or computed tomography (CT) scans. In some embodiments, the response to treatment according to the methods provided herein is assessed using the 2014 Lugano Criteria (Cheson et al., (2014) J Clin Oncol 32:3059-3068). In some embodiments, the response to treatment according to the methods provided herein is assessed during treatment or at the end of treatment. In some embodiments, the response to treatment according to the methods provided herein is assessed in a patient in comparison to assessments prior to administration of treatment according to the methods provided herein. In some embodiments, CT scans are performed every 6 months. In some embodiments, PET scans are performed before and at the end of administration of treatment according to the methods provided herein. In some embodiments, PET and/or CT (e.g., PET-CT) scans include the skull-base to mid-thigh. In some embodiments, full-body PET-CT scans are performed. In some embodiments, CT scans include intravenous contrast, which can include, without limitation, chest, neck, abdomen, and pelvic scans. In some embodiments, radiographic assessments of tumors are performed, for example, if disease progression or relapse is suspected. In some embodiments, bone response to treatment according to the methods provided herein is assessed using bone marrow biopsies, which can be performed according to any method known in the art. In some cases, bone marrow biopsies are performed in patients with negative bone signal on PET-CT. In some embodiments, PET-CT and/or CT scans are obtained prior to administration of treatment according to the methods provided herein, and as clinically indicated during and after administration of treatment according to the methods provided herein. In some embodiments, PET-CT and/or CT scans are performed for up to two years after administration of treatment according to the methods provided herein.

In some embodiments, administration of the immunoconjugate, the rituximab, the gemcitabine, and the oxaliplatin results in a complete response in the human. In some embodiments, a complete response comprises a complete metabolic response based on PET-CT, according to Lugano 2014 response criteria (Cheson et al., (2014) J Clin Oncol 32:3059-3068). In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the complete response rate (CRR), e.g., as compared to administration of an anti-CD20 antibody (e.g., rituximab) and one or more chemotherapeutic agents (e.g., gemcitabine and oxaliplatin). In some embodiments, CRR refers to the proportion of patients who achieve complete metabolic response at the end of treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., based on positron emission tomography and computed tomography (PET-CT) analyses according to the Lugano 2014 response criteria. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the CRR of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, the CRR is determined without including PET data. In some embodiments, administration of the immunoconjugate, the rituximab, the gemcitabine, and the oxaliplatin to a plurality of humans results in an increase in the complete response rate (CRR), e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, which may be combined with any of the preceding embodiments, administration of the immunoconjugate, the rituximab, the gemcitabine, and the oxaliplatin to a plurality of humans results in a complete response rate of at least about 35% or greater, at least about 40% or greater, at least about 50% or greater, at least about 60% or greater, at least about 70% or greater, at least about 80% or greater, at least about 90% or greater, at least about 95% or greater, or about 100% of the humans in the plurality.

In some embodiments, administration of the immunoconjugate, the rituximab, the gemcitabine, and the oxaliplatin results in a partial response in the human. In some embodiments, a partial response comprises a partial metabolic response including PET CT data, according to Lugano 2014 response criteria. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the objective response rate (ORR), e.g., as compared to administration of an anti-CD20 antibody (e.g., rituximab) and one or more chemotherapeutic agents (e.g., gemcitabine and oxaliplatin). In some embodiments, ORR refers to the proportion of patients who achieve complete or partial metabolic responses at the end of treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., based on positron emission tomography and computed tomography (PET-CT) analyses according to the Lugano 2014 response criteria. In some embodiments, ORR refers to the proportion of patients who achieve complete or partial metabolic responses at the end of treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., according to the Lugano 2014 response criteria. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the ORR of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, ORR is determined without including PET data. In some embodiments, administration of the immunoconjugate, the rituximab, the gemcitabine, and the oxaliplatin to a plurality of humans results in an increase in the objective response rate (ORR), e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, which may be combined with any of the preceding embodiments, administration of the immunoconjugate, the rituximab, the gemcitabine, and the oxaliplatin to a plurality of humans results in an objective response rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, which may be combined with any of the preceding embodiments, administration of the immunoconjugate, the rituximab, the gemcitabine, and the oxaliplatin to a plurality of humans results in a partial response rate of at least about 10% or greater, at least about 20% or greater, at least about 30% or greater, at least about 40% or greater, at least about 50% or greater, at least about 60% or greater, at least about 70% or greater, at least about 80% or greater, at least about 90% or greater, at least about 95% or greater, or about 100% of the humans in the plurality. In some embodiments, which may be combined with any of the preceding embodiments, administration of the immunoconjugate, the rituximab, the gemcitabine, and the oxaliplatin to a plurality of humans results in an increase in the partial response rate, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, the partial response rate refers to the proportion of patients who achieve partial metabolic responses at the end of treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., based on positron emission tomography and computed tomography (PET-CT) analyses according to the Lugano 2014 response criteria. In some embodiments, the partial response rate refers to the proportion of patients who achieve partial metabolic responses at the end of treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., according to the Lugano 2014 response criteria.

In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the rate of best overall response (BOR), e.g., as compared to administration of an anti-CD20 antibody (e.g., rituximab) and one or more chemotherapeutic agents (e.g., gemcitabine and oxaliplatin). In some embodiments, BOR refers to the best response during treatment with the immunoconjugate (e.g., polatuzumab vedotin-piiq), the anti-CD20 antibody (e.g., rituximab), and the one or more chemotherapeutic agents (e.g., gemcitabine and oxaliplatin), e.g., based on positron emission tomography and computed tomography (PET-CT) or CT analyses according to the Lugano 2014 response criteria. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the rate of BOR of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, administration of the immunoconjugate, the rituximab, the gemcitabine, and the oxaliplatin to a plurality of humans results in an increase in the best overall response rate (BOR), e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin.

In some embodiments, administration of the immunoconjugate, the rituximab, the gemcitabine, and the oxaliplatin results in event-free survival of the human. In some embodiments, administration of the immunoconjugate, the rituximab, the gemcitabine, and the oxaliplatin results in event-free survival of the human for at least about 4 months, at least about 5 months, at least about 6 months, at least about 7 months, at least about 8 months, at least 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, or at least about 25 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 4 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 5 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 6 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 9.5 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 11 months after the start of treatment with the immunoconjugate, the rituximab, the gemcitabine, and the oxaliplatin. In some embodiments, administration of the immunoconjugate, the rituximab, the gemcitabine, and the oxaliplatin results in event-free survival of the human for at least about 14 months after the start of treatment with the immunoconjugate, the rituximab, the gemcitabine, and the oxaliplatin. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the event-free survival (EFS_{eff}) time, e.g., as compared to administration of an anti-CD20 antibody (e.g., rituximab) and one or more chemotherapeutic agents (e.g., gemcitabine and oxaliplatin). In some embodiments, EFS_{eff} refers to the time from the first administration of the immunoconjugate (e.g., polatuzumab vedotin-piiq), the anti-CD20 antibody (e.g., rituximab), and the one or more chemotherapeutic agents (e.g., gemcitabine and oxaliplatin) to the time of an occurrence of any of disease progression or relapse, death due to any cause, or initiation of another anti-lymphoma treatment. In some embodiments, EFS_{eff} is measured in days, weeks, months, or years. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in EFS_{eff} of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, the methods used for analysis of PFS (e.g., as described above) are used to analyze EFS_{eff}. In some embodiments, event-free survival is measured from the time of first administration of the immunoconjugate, the rituximab, the gemcitabine, and the oxaliplatin to the first occurrence of any of (i) Disease progression or relapse (based on response including PET CT data or not including any PET data); (ii) Death due to any cause; or (iii) Initiation of a new anti-lymphoma treatment (NALT). In some embodiments, administration of the immunoconjugate, the rituximab, the gemcitabine, and the oxaliplatin results in an increase in event-free survival of the human, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to administration of rituximab, gemcitabine, and oxaliplatin to a corresponding human. In some embodiments, administration of the immunoconjugate, the rituximab, the gemcitabine, and the oxaliplatin to a plurality of humans results in an increase in event-free survival of the humans in the plurality, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, which may be combined with any of the preceding embodiments, administration of the immunoconjugate, the rituximab, the gemcitabine, and the oxaliplatin to a plurality of humans results in a 2-year event-free survival rate of at least about 44% or greater, at least about 45% or greater, at least about 50% or greater, at least about 60% or greater, at least about 70% or greater, at least about 80% or greater, at least about 90% or greater, at least about 95% or greater, or about 100% of the humans in the plurality. In some embodiments, administration of the immunoconjugate, the rituximab, the gemcitabine, and the oxaliplatin to a plurality of humans results in an increase in the 1-year, 2-year, 3.5-year, or 5-year event-free survival rate, e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin. In some embodiments, a 1-year event-free survival rate refers to the proportion of humans in a plurality of humans administered the immunoconjugate, the rituximab, the gemcitabine, and the oxaliplatin that do not have an occurrence of any of (i) Disease progression or relapse (based on response including PET CT data or not including any PET data), (ii) Death due to any cause, or (iii) Initiation of a new anti-lymphoma treatment (NALT) at 1 year after the start of treatment with the immunoconjugate, the rituximab, the gemcitabine, and the oxaliplatin. In some embodiments, a 2-year event-free survival rate refers to the proportion of humans in a plurality of humans administered the immunoconjugate, the rituximab, the gemcitabine, and the oxaliplatin that do not have an occurrence of any of (i) Disease progression or relapse (based on response including PET CT data or not including any PET data), (ii) Death due to any cause, or (iii) Initiation of a new anti-lymphoma treatment (NALT) at 2 years after the start of treatment with the immunoconjugate, the rituximab, the gemcitabine, and the oxaliplatin. In some embodiments, a 3.5-year event-free survival rate refers to the proportion of humans in a plurality of humans administered the immunoconjugate, the rituximab, the gemcitabine, and the oxaliplatin that do not have an occurrence of any of (i) Disease progression or relapse (based on response including PET CT data or not including any PET data), (ii) Death due to any cause, or (iii) Initiation of a new anti-lymphoma treatment (NALT) at 3.5 years after the start of treatment with the immunoconjugate, the rituximab, the gemcitabine, and the oxaliplatin. In some embodiments, a 5-year event-free survival rate refers to the proportion of humans in a plurality of humans administered the immunoconjugate, the rituximab, the gemcitabine, and the oxaliplatin that do not have an occurrence of any of (i) Disease progression or relapse (based on response including PET CT data or not including any PET data), (ii) Death due to any cause, or (iii) Initiation of a new anti-lymphoma treatment (NALT) at 5 years after the start of treatment with the immunoconjugate, the rituximab, the gemcitabine, and the oxaliplatin.

In some embodiments, event-free survival is measured from the time of first administration of the immunoconjugate, the rituximab, the gemcitabine, and the oxaliplatin, or from the start of treatment with the immunoconjugate, the rituximab, the gemcitabine, and the oxaliplatin, to the first occurrence of any of (i) Disease progression or relapse (based on response including PET CT data or not including any PET data); (ii) Death due to any cause; or (iii) Initiation of a new anti-lymphoma treatment (NALT).

In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in the time of duration of response (DOR), e.g., as compared to administration of an anti-CD20 antibody (e.g., rituximab) and one or more chemotherapeutic agents (e.g., gemcitabine and oxaliplatin). In some embodiments, DOR is measured in patients administered treatment according to the methods provided herein, who have an objective response using the Lugano 2014 Criteria from the date of the first occurrence of a complete or partial response until the first date that progressive disease or death occur. In some embodiments, DOR is measured in days, weeks, months, or years. In some embodiments, administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein results in an increase in DOR of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, e.g., as compared to administration of an anti-CD20 antibody (e.g., rituximab) and one or more chemotherapeutic agents (e.g., gemcitabine and oxaliplatin). In some embodiments, the methods used for analysis of PFS (e.g., as described above) are used to analyze DOR, except that DOR analysis is not stratified. In some embodiments, administration of the immunoconjugate, the rituximab, the gemcitabine, and the oxaliplatin to a plurality of humans results in an increase in the duration of response (DOR), e.g., of any of at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, or at least about 6 fold, or more, compared to a corresponding plurality of humans administered rituximab, gemcitabine, and oxaliplatin.

In some embodiments, response to treatment with the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein is assessed using patient-reported outcome (PRO) instruments. Examples of PRO instruments include, without limitation, FACT/GOG-Ntx12 neuropathy (Kopec et al., (2006) J Supportive Oncol, 4:W1-W8; Calhoun et al., (2003) Int J Gynecol Cancer, 13:741-748), EQ-5D-5L (EuroQol (1990) Health Policy, 16:199-208; Brooks (1996) Health Policy, 37:53-72; Herdman et al., (2011) 20:1727-1736; Janssen et al., (2013) Qual Life Res, 22:1717-1727; http://www(dot)euroqol(dot)org/about-eq-5d/valuation-of-eq-5d; Devlin et al., (2017) Health Economics, 1-16), EORTC QLQ-C30 (Aaronson et al., (1993) J Natl Cancer Inst, 85:365-376; Fitzsimmons et al., (1999) 35:939-941), and FACT/Lym (Cella et al., (1993) J Clin ONcol, 11:570-579; Cella et al., (2005) Blood, 106:750; Carter et al., (2008) Blood, 112:2376; Hlubocky et al., (2013) Lymphoma, ID147176; Webster et al., (2003) Health Qual Life Outcomes, 1:79).

In some embodiments, adverse events after administration of the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein are assessed according to the National Cancer Institute Common Terminology Criteria for Adverse Events, Version 5 (NCI CTCAE v5.0), Functional Assessment of Cancer Therapy/Gynecologic Oncology Group-Neurotoxicity 12-Item Scale (FACT/GOG-Ntx12), clinical laboratory test results, electrocardiograms (ECG), and/or vital signs. In some embodiments, adverse events leading to withdrawal of treatment, adverse events leading to dose reduction or interruption, Grade ≥3 adverse events, adverse events leading to death, serious adverse events, and/or adverse events of special interest are analyzed. In some embodiments, adverse events as provided herein are analyzed with respect to exposure to the immunoconjugate (e.g., polatuzumab vedotin-piiq), the anti-CD20 antibody (e.g., rituximab), and/or the one or more chemotherapeutic agents (e.g., gemcitabine and oxaliplatin).

In some embodiments, occurrences of peripheral neuropathy in patients administered the immunoconjugate (e.g., polatuzumab vedotin-piiq), the anti-CD20 antibody (e.g., rituximab), and the one or more chemotherapeutic agents (e.g., gemcitabine and oxaliplatin), according to the methods provided herein, are assessed by FACT/GOG-Ntx12 score and/or by NCI CTCAE v5.0. Symptoms of peripheral neuropathy (sensory and/or motor) include, but are not limited to hypoesthesia, hyperesthesia, paresthesia, dysesthesia, discomfort, a burning sensation, weakness, gait disturbance, or neuropathic pain. In some embodiments, the rate of occurrences of peripheral neuropathy, e.g., based on adverse event analysis, is calculated. In some embodiments, all patients with an occurrence of peripheral neuropathy are followed up after termination of treatment according to the methods provided herein until resolution or stabilization. In some embodiments, less than 50% (e.g., less than any of about 50%, 45%, 40%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0.5%) of patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein experience a ≥Grade 3 (e.g., any of Grade 3 or greater, Grade 4 or greater, or Grade 5 or greater) peripheral neuropathy that does not resolve to ≤Grade 1 within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, less than about 40% of patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein experience a ≥Grade 3 (e.g., any of Grade 3 or greater, Grade 4 or greater, or Grade 5 or greater) peripheral neuropathy that does not resolve to ≤Grade 1 within 14 days. In some embodiments, less than about 33% of patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein experience a ≥Grade 3 (e.g., any of Grade 3 or greater, Grade 4 or greater, or Grade 5 or greater) peripheral neuropathy that does not resolve to ≤Grade 1 within 14 days. In some embodiments, less than about 30% of patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein experience a ≥Grade 3 (e.g., any of Grade 3 or greater, Grade 4 or greater, or Grade 5 or greater) peripheral neuropathy that does not resolve to ≤Grade 1 within 14 days. In some embodiments, administration of the immunoconjugate, the rituximab, the gemcitabine, and the oxaliplatin does not result in peripheral neuropathy of Grade 4 or greater (e.g., any of Grade 4 or greater, or Grade 5 or greater) in the human. In some embodiments, after administration of the immunoconjugate, the rituximab, the gemcitabine, and the oxaliplatin, the human does not experience peripheral neuropathy of Grade 4 or greater (e.g., any of Grade 4 or greater, or Grade 5 or greater). In some embodiments, after administration of the immunoconjugate, the rituximab, the gemcitabine, and the oxaliplatin, the human does not experience neurotoxicity of Grade 4 or greater (e.g., any of Grade 4 or greater, or Grade 5 or greater). In some embodiments, neurotoxicity refers to sensory and/or motor peripheral neuropathy. In some embodiments, administration of the immunoconjugate, the rituximab, the gemcitabine, and the oxaliplatin to a plurality of humans results in 33% or fewer (e.g., any of about 33% or fewer, about 32% or fewer, about 31% or fewer, about 30% or fewer, about 29% or fewer, about 28% or fewer, about 27% or fewer, about 26% or fewer, about 25% or fewer, about 24% or fewer, about 23% or fewer, about 22% or fewer, about 21% or fewer, about 20% or fewer, about 19% or fewer, about 18% or fewer, about 17% or fewer, about 16% or fewer, about 15% or fewer, about 14% or fewer, about 13% or fewer, about 12% or fewer, about 11% or fewer, about 10% or fewer, about 9% or fewer, about 8% or fewer, about 7% or fewer, about 6% or fewer, about 5% or fewer, about 4% or fewer, about 3% or fewer, about 2% or fewer, about 1% or fewer, or about 0.5% or fewer) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In some embodiments, administration of the immunoconjugate, the rituximab, the gemcitabine, and the oxaliplatin to a plurality of humans results in less than about 40% (e.g., less than any of about 40%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0.5%) of the humans in the plurality experiencing peripheral neuropathy of Grade 3 or greater (e.g., any of Grade 3 or greater, Grade 4 or greater, or Grade 5 or greater) that does not resolve to Grade 1 or lower within 14 days (e.g., within any of 14 days, 13 days, 12 days, 11 days, 10 days, 9 days, 8 days, 7 days, 6 days, 5 days, 4 days, 3 days, 2 days, or 1 day). In certain embodiments, the immunoconjugate, the rituximab, the gemcitabine, and the oxaliplatin have been administered for at least four 21-day cycles.

In some embodiments, occurrences of drug-induced liver injury are assessed.

In some embodiments, the immunogenicity of the immunoconjugate (e.g., polatuzumab vedotin-piiq) when administered according to the methods provided herein is assessed. In some embodiments, the immunogenicity of the immunoconjugate (e.g., polatuzumab vedotin-piiq) when administered according to the methods provided herein is assessed by measuring anti-drug antibodies (ADA) against the immunoconjugate. In some embodiments, ADAs are measured using a validated antibody-bridging enzyme-linked immunosorbent assay (ELISA) in patient serum samples.

In some embodiments, the incidence of adverse events in patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein is assessed after completion of at least one 21-day cycle of treatment. In some embodiments, the incidence of adverse events in patients administered the immunoconjugate (e.g., polatuzumab vedotin-piiq), the anti-CD20 antibody (e.g., rituximab), and the one or more chemotherapeutic agents (e.g., gemcitabine and oxaliplatin) according to the methods provided herein is assessed after completion of at least two 21-day cycles of treatment.

In some embodiments, the doses of the immunoconjugate (e.g., polatuzumab vedotin-piiq), the anti-CD20 antibody (e.g., rituximab), and/or the one or more chemotherapeutic agents (e.g., gemcitabine and oxaliplatin) are modified according to the occurrence of adverse events. In some embodiments, adverse events are based on laboratory test results obtained within 72 hours before infusion of Day 1 of a cycle. In certain embodiments, symptoms are graded according to the National Cancer Institute Common Terminology Criteria for Adverse Events, Version 5 (NCI CTCAE v5.0).

In some embodiments, administration of the immunoconjugate, the rituximab, the gemcitabine, and the oxaliplatin does not result in neutropenia in the human. In some embodiments, the methods provided herein further comprise administering a prophylactic treatment for neutropenia to the human before, during, and/or after administration of the immunoconjugate, the rituximab, the gemcitabine, and the oxaliplatin to the human. In some embodiments, the prophylactic treatment for neutropenia comprises administration of G-CSF, e.g., in each cycle of treatment. In some embodiments, if Grade 3 or Grade 4 neutropenia occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is held until absolute neutrophil count (ANC) recovers to >1000/μL. In some embodiments, growth factors (e.g., G-CSF) are administered if necessary to manage neutropenia. See, e.g., Smith et al (2016) J Clin Oncol, 24:3187-205. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is resumed without any additional dose reductions if ANC recovers to >1000/μL in the human on or before Day 7. In some embodiments, the dose of polatuzumab vedotin-piiq is reduced to 1.4 mg/kg if ANC recovers to >1000/μL in the human after Day 7. In some embodiments, if a prior dose reduction of polatuzumab vedotin-piiq has occurred, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, if Grade 3 or Grade 4 thrombocytopenia occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is held until platelets recover to >75,000/μL. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is resumed without any additional dose reductions if platelets recover to >75,000/μL on or before Day 7. In some embodiments, the dose of polatuzumab vedotin-piiq is reduced to 1.4 mg/kg if platelets recover to >75,000/μL after Day 7. In some embodiments, if a prior dose reduction of polatuzumab vedotin-piiq has occurred, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, if Grade 2 or Grade 3 peripheral neuropathy occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is delayed until improvement to ≤Grade 1. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is restarted with polatuzumab vedotin-piiq at a permanently reduced dose of 1.4 mg/kg and oxaliplatin at a dose of 75 mg/m² if the Grade 2 or Grade 3 peripheral neuropathy recovers to Grade ≤1 within ≤14 days. In some embodiments, treatment with polatuzumab vedotin-piiq alone is discontinued if the Grade 2 or Grade 3 peripheral neuropathy does not recover to Grade ≤1 on or before Day 14 if the patient previously had Grade 2 peripheral neuropathy and/or if a prior dose reduction to 1.4 mg/kg for polatuzumab vedotin-piiq or 75 mg/m² for oxaliplatin occurred. In some embodiments, treatment is discontinued if the subject had a previous Grade 3 peripheral neuropathy. In some embodiments, oxaliplatin and polatuzumab vedotin-piiq are permanently discontinued if the Grade 2 or Grade 3 peripheral neuropathy does not recover to ≤Grade 1 until >14 days or after the scheduled date for the next cycle. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued if a Grade 4 peripheral neuropathy occurs.

In some embodiments, if pharyngolaryngeal dysesthesia occurs, oxaliplatin infusion is prolonged to 6 hours.

In some embodiments, if a Grade 1-2 infusion related reaction (IRR) occurs, infusion is slowed or held. In some embodiments, subjects are given supportive treatment. In some embodiments, supportive treatment includes acetaminophen/paracetamol and an antihistamine such as diphenhydramine and/or intravenous saline. In some embodiments, supportive treatment for bronchospasm, urticaria, or dyspnea includes antihistamines, oxygen, corticosteroids (e.g., 100 mg IV prednisolone or equivalent), and/or bronchodilators. In some embodiments, upon resolution of symptoms, infusion-rate escalation is resumed. In some embodiments, for infusion rate escalation after re-initiation upon complete resolution of symptoms, the infusion is resumed at 50% of the rate achieved prior to interruption. In some embodiments, in the absence of infusion-related symptoms, the rate of infusion is escalated in increments of 50 mg/hour every 30 minutes. In some embodiments, if a Grade 1-2 IRR occurs, polatuzumab vedotin is infused over 90 minutes in the next cycle. In some embodiments, if no infusion-related reaction occurs, subsequent infusions of polatuzumab vedotin are administered over 30 minutes. In some embodiments, premedication is administered for all cycles. In some embodiments, if wheezing or urticaria symptoms recur, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, if a Grade 3 IRR occurs, infusion is discontinued. In some embodiments, subjects are given supportive treatmnet. In some embodiments, supportive treatment includes acetaminophen/paracetamol and an antihistamine such as diphenhydramine and/or intravenous saline. In some embodiments, supportive treatment for bronchospasm, urticaria, or dyspnea includes antihistamines, oxygen, corticosteroids (e.g., 100 mg IV prednisolone or equivalent), and/or bronchodilators. In some embodiments, upon resolution of symptoms, infusion-rate escalation is resumed. In some embodiments, for infusion rate escalation after re-initiation upon complete resolution of symptoms, the infusion is resumed at 50% of the rate achieved prior to interruption. In some embodiments, in the absence of infusion-related symptoms, the rate of infusion is escalated in increments of 50 mg/hour every 30 minutes. In some embodiments, if the same adverse event recurs with the same severity, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, if a Grade 4 IRR occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, if total bilirubin >3.0 mg/dL is observed, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is delayed until resolution to ≤1.5 mg/dL within ≤14 days. In some embodiments, if an increase in hepatic transaminase >3×baseline and an increase in direct bilirubin >2×ULN without any findings of cholestasis or jaundice or signs of hepatic dysfunction and in the absence of other contributory factors (e.g., worsening of metastatic disease or concomitant exposure to known hepatotoxic agent or of a documented infectious etiology) are observed, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, administration of the immunoconjugate, the rituximab, the gemcitabine, and the oxaliplatin does not result in tumor lysis syndrome in the human. In some embodiments, the methods provided herein further comprise administering a prophylactic treatment for tumor lysis syndrome to the human before, during, and/or after administration of the immunoconjugate, the rituximab, the gemcitabine, and the oxaliplatin to the human. In some embodiments, the prophylactic treatment comprises hydration, e.g., 3 liters of fluids per day, e.g., starting at about 1 or 2 days prior to administration of the immunoconjugate, the rituximab, the gemcitabine, and the oxaliplatin. In some embodiments, the prophylactic treatment comprises allopurinol (e.g., 300 mg/day orally) or a suitable alternative treatment (e.g., rasburicase), starting at about 48 to about 72 hours prior to administration of the immunoconjugate, the rituximab, the gemcitabine, and the oxaliplatin. In some embodiments, the prophylactic treatment comprises hydration, e.g., 3 liters of fluids per day, e.g., starting at about 1 or 2 days prior to administration of the immunoconjugate, the rituximab, the gemcitabine, and the oxaliplatin; and allopurinol (e.g., 300 mg/day orally) or a suitable alternative treatment (e.g., rasburicase), starting at about 48 to about 72 hours prior to administration of the immunoconjugate, the rituximab, the gemcitabine, and the oxaliplatin. In some embodiments, the prophylactic treatment for tumor lysis syndrome is administered to the human if the human has a risk of tumor lysis syndrome, e.g., if the human has a high tumor burden (e.g., lymphocyte count ≥25×10⁹/L or bulky lymphadenopathy). In some embodiments, the prophylactic treatment for tumor lysis syndrome is administered prior to each administration of the immunoconjugate, the rituximab, the gemcitabine, and the oxaliplatin. In some embodiments, if Grade 3 or Grade 4 tumor lysis syndrome (TLS) occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is held. In some embodiments, the next dose is delayed for up to 14 days. In some embodiments, upon complete resolution of TLS, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is resumed at the full dose during the next scheduled infusion in conjuction with prophylactic therapy.

In some embodiments, administration of the immunoconjugate, the rituximab, the gemcitabine, and the oxaliplatin does not result in an infection in the human. In some embodiments, the infection is a pneumocystis infection or a herpesvirus infection. In some embodiments, the methods provided herein further comprise administering a prophylactic treatment for infections to the human before, during, and/or after administration of the immunoconjugate, the rituximab, the gemcitabine, and the oxaliplatin to the human. In some embodiments, the prophylactic treatment for infections comprises one or more suitable anti-viral medications. In some embodiments, the human is administered a prophylactic treatment for hepatitis B reactivation, e.g., as described in Flowers et al., 2013; National Comprehensive Cancer Network (NCCN) 2017.

In some embodiments, if Grade 3 or 4 non-hematologic toxicity (excluding alopecia, nausea, and vomiting) occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is delayed for up to 14 days. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is resumed at a reduced dose or at the full dose if improvement to Grade ≤1 or baseline is observed.

In some embodiments, if Grade 2 non-hematologic toxicity occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is delayed for up to 14 days. In some embodiments, previous doses of treatment with the immunoconjugate (e.g., polatuzumab vedotin-piiq), the anti-CD20 antibody (e.g., rituximab), and the one or more chemotherapeutic agents (e.g., gemcitabine and oxaliplatin) are administered if improvement to Grade ≤1 or baseline is observed.

In some embodiments, if Grade 1 non-hematologic toxicity occurs, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is not modified.

In some embodiments, administration of the immunoconjugate, the rituximab, the gemcitabine, and the oxaliplatin does not result in Hepatitis B reactivation in the human. In some embodiments, the methods provided herein further comprise administering a prophylactic treatment for Hepatitis B reactivation to the human before, during, and/or after administration of the immunoconjugate, the rituximab, the gemcitabine, and the oxaliplatin to the human. In some embodiments, the prophylactic treatment for Hepatitis B reactivation comprises an anti-viral medication, e.g., as described in in Flowers et al., 2013; National Comprehensive Cancer Network (NCCN) 2017. In some embodiments, the methods provided herein further comprise administering anti-viral medication to the human if Hepatitis B reactivation is detected in the human, e.g., a suitable nucleoside analogue. In some embodiments, Hepatitis B reactivation is determined by new detectable HBV-DNA levels. In some embodiments, if HBV-DNA levels are between the World Health Organization (WHO)-recommended range of 29 IU/mL and 100 IU/mL, HBV-DNA levels are retested within 2 weeks. In some embodiments, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is held and the subject is treated with a nucleoside analogue if HBV-DNA levels are positive. In some embodiments, if HBV-DNA levels at the WHO-recommended cutoff of >100 IU/mL are observed, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is held and a nucleoside analogue is administered. In some embodiments, if rising HBV-DNA viral load (exceeding 100 IU/mL) while on an appropriate anti-viral therapy is observed, treatment with the immunoconjugate (e.g., polatuzumab vedotin-piiq), the anti-CD20 antibody (e.g., rituximab), and the one or more chemotherapeutic agents (e.g., gemcitabine and oxaliplatin) is discontinued.

In some embodiments, administration of the immunoconjugate, the rituximab, the gemcitabine, and the oxaliplatin does not result in one or more adverse events selected from the group consisting of drug-induced liver injury, progressive multifocal leukoencephalopathy, systemic hypersensitivity reaction, anaphylactic reaction, anaphylactoid reaction, and second malignancy. In some embodiments, drug-induced liver injury comprises elevated ALT or AST in combination with either an elevated bilirubin or clinical jaundice, as defined by Hy's Law, in the human. In some embodiments, drug-induced liver injury comprises treatment-emergent ALT or AST >3×baseline value in combination with total bilirubin >2×ULN (of which ≥35% is direct bilirubin) in the human. In some embodiments, drug-induced liver injury comprises treatment-emergent ALT or AST >3×baseline value in combination with clinical jaundice in the human. In some embodiments, systemic hypersensitivity reactions, anaphylactic reactions, and anaphylactoid reactions are assessed using Sampson's criteria.

Provided is an immunoconjugate comprising the formula

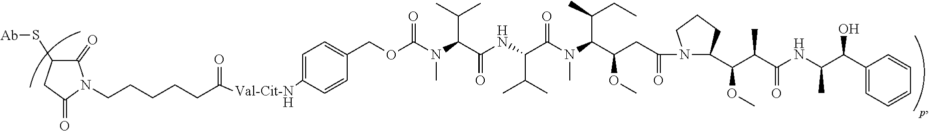

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL, e.g., relapsed/refractory DLBCL), in an individual (a human individual) in need thereof. In some embodiments, the immunoconjugate is for use according to any of the methods provided herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin-piiq.

Also provided is the use of an immunoconjugate comprising the formula

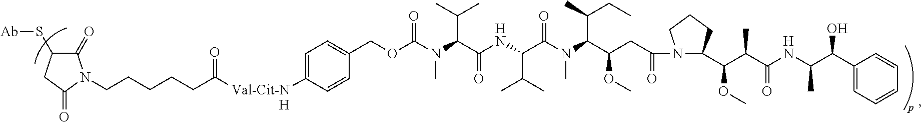

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 in the manufacture of a medicament for treating diffuse large B-cell lymphoma (DLBCL, e.g., relapsed/refractory DLBCL), in an individual (a human individual) in need thereof, wherein the medicament is for (e.g., formulated for) administration in combination with an anti-CD20 antibody (e.g., rituximab), and one or more chemotherapeutic agents (e.g., gemcitabine and oxaliplatin). In some embodiments, the medicament (i.e., the medicament comprising the immunoconjugate) is for use in a method described herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin-piiq.

Provided is an immunoconjugate comprising the formula

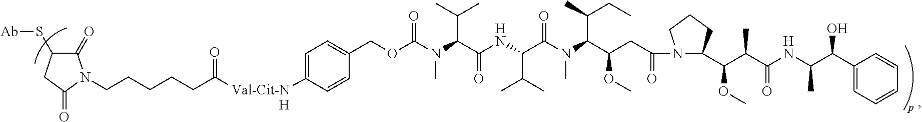

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in a method of treating diffuse large B-cell lymphoma (DLBCL, e.g., relapsed/refractory DLBCL), in an individual (a human individual) in need thereof, the method comprising administering to the individual an effective amount of (a) the immunoconjugate, (b) rituximab (c) one or more chemotherapeutic agents (e.g., gemcitabine and oxaliplatin), wherein the immunoconjugate is administered at a dose between about 1.4 and about 1.8 mg/kg, the rituximab is administered at a dose 375 mg/m², and the one or more chemotherapeutic agents are administered at a dose of between 50-2000 mg/m² (e.g., gemcitabine administered at a dose of 1000 mg/m² and oxaliplatin administered at a dose of 100 mg/m²). In some embodiments, the immunoconjugate is for use according to a method described herein. In some embodiments, p is between 3 and 4. In some embodiments, p is 3.5. In some embodiments, the immunoconjugate comprises an anti-CD79b antibody comprising a heavy chain comprising the amino acid sequence of SEQ ID NO: 36 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin-piiq.

Also provided is an immunoconjugate comprising the formula

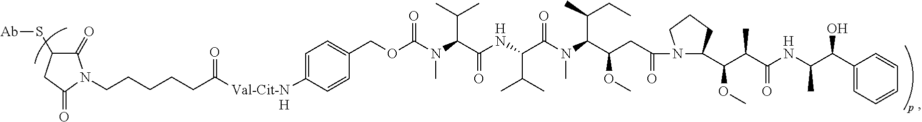

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in the manufacture of a medicament for treating diffuse large B-cell lymphoma (DLBCL, e.g., relapsed/refractory DLBCL), in an individual (a human individual) in need thereof, wherein the medicament is for (e.g., formulated for) administration in combination with rituximab, and one or more chemotherapeutic agents (e.g., gemcitabine and oxaliplatin) wherein the medicament is formulated for administration of the immunoconjugate at a dose between about 1.4 and about 1.8 mg/kg, the rituximab is for administration at a dose of 375 mg/m², and the one or more chemotherapeutic agents are for administration at a dose of 50-2000 mg/m² (e.g., gemcitabine administered at a dose of 1000 mg/m² and oxaliplatin administered at a dose of 100 mg/m²). In some embodiments, the medicament (i.e., the medicament comprising the immunoconjugate) is for use according to a method described herein. In some embodiments, p is between 3 and 4. In some embodiments, p is 3.5. In some embodiments, the immunoconjugate comprises an anti-CD79b antibody comprising a heavy chain comprising the amino acid sequence of SEQ ID NO: 36 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin-piiq.

### IV. Immunoconjugates Comprising an Anti-CD79b Antibody and a Drug/Cytotoxic Agent (“Anti-CD79b Immunoconjugates”)

In some embodiments, the anti-CD79b immunoconjugate comprises an anti-CD79b antibody (Ab) which targets a cancer cell (such as a diffuse large B-cell lymphoma (DLBCL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some embodiments, the immunoconjugate comprises the formula Ab-(L-D)p, wherein: (a) Ab is the anti-CD79b antibody; (b) L is a linker; (c) D is a cytotoxic agent; and (d) p ranges from 1-8.

An exemplary anti-CD79b immunoconjugate comprises Formula I:

Ab−(L−D)p (I)

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

A. Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugate can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, a-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

In some embodiments, the linker is a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker (L) has the following Formula II:

-Aₐ-W_{w}—Y_{y}- (II)

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

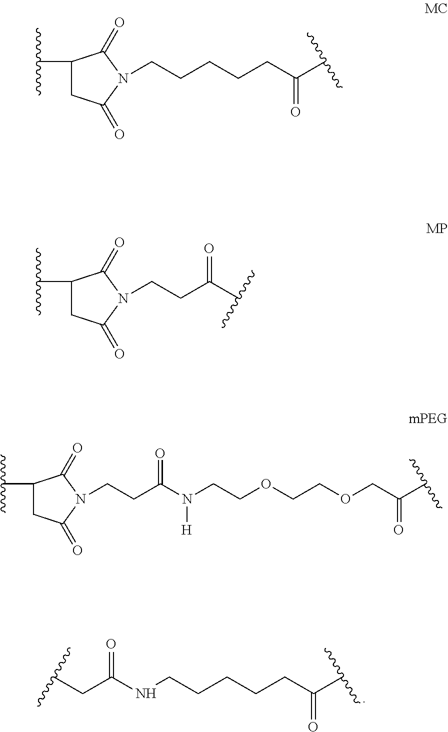 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

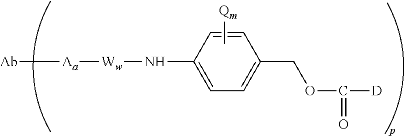

wherein Q is —C₁-C₈ alkyl, —O—(C₁-C₈ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the a-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an anti-CD79 immunoconjugates of Formulas III, IV, V:

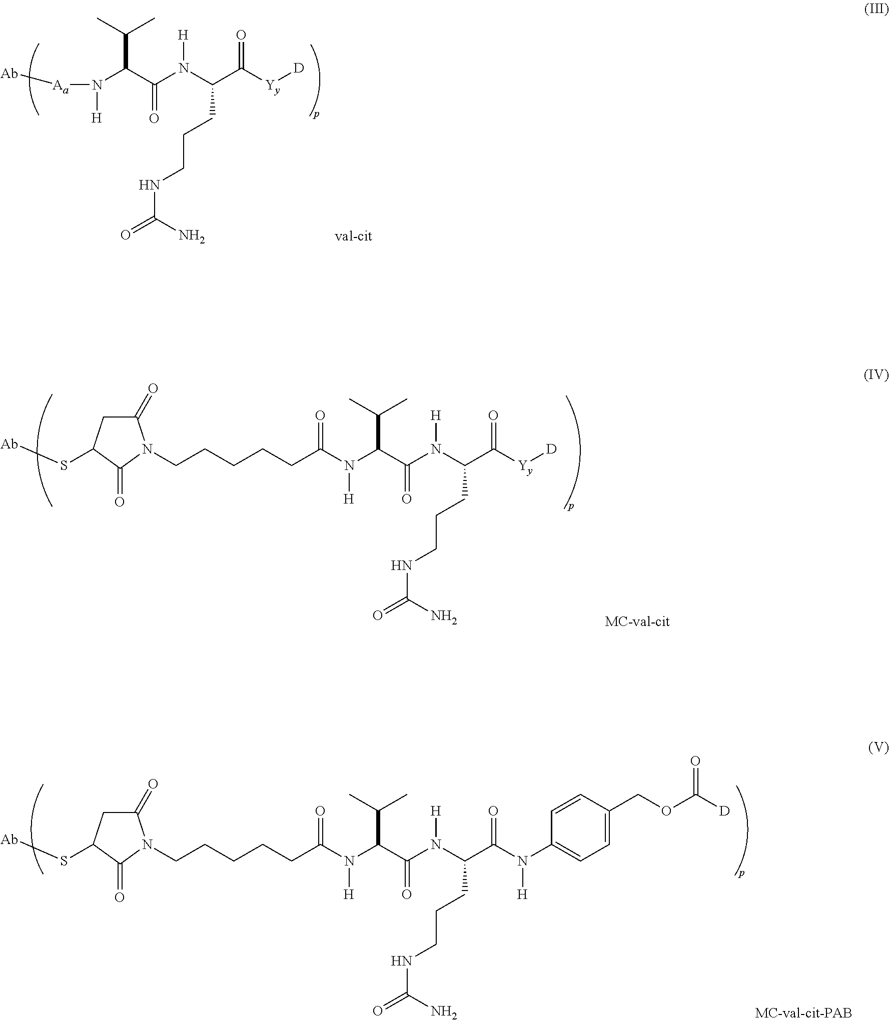

wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15, 1 to 10, 1 to 8, 2 to 5, or 3 to 4).

In some embodiments, the anti-CD79b immunoconjugate comprises a structure of any one of formulas VI-V below:

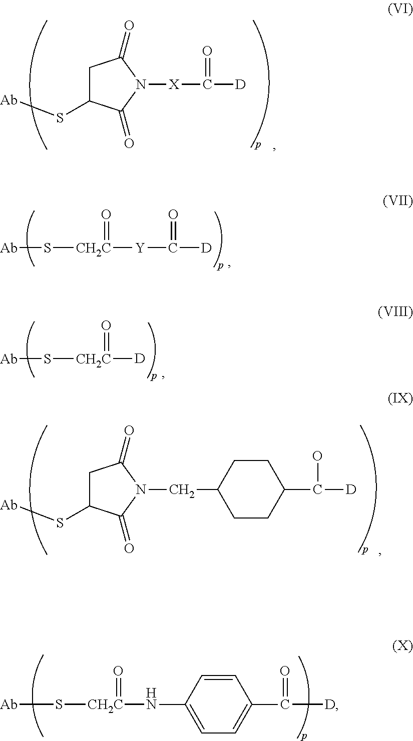

where X is:

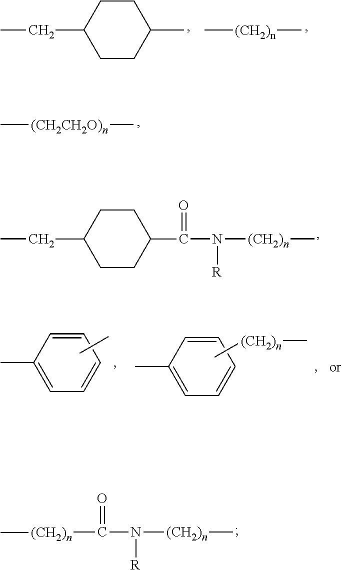

Y is:

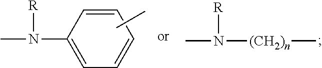

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a nonlimiting example, a charged substituent such as sulfonate (—SO₃-) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion)^{a} is coupled to drug/cytotoxic agent-(linker portion)^{b} to form the anti-CD79b immunoconjugate of Formula I. In some such embodiments, the anti-CD79b antibody comprises more than one (linker portion)^{a} substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

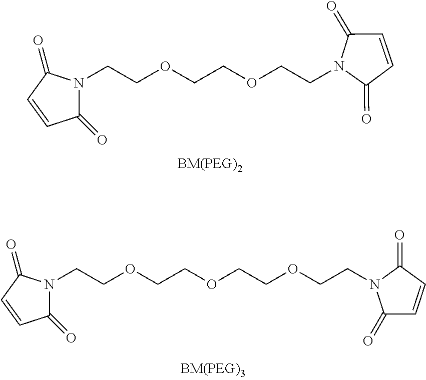 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, Ill.), Molecular Biosciences Inc. (Boulder, Colo.), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

B. Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some such embodiments, the immunoconjugate comprises an anti-CD79b antibody comprising at least one of: (i) HVR-H3 comprising the amino acid sequence of SEQ ID NO: 23, and/or (ii) HVR-L1 comprising an amino acid sequence of SEQ ID NO: 24. In some embodiments, the immunoconjugate comprises an anti-CD79b antibody comprising at least one of: (i) HVR-H3 comprising the amino acid sequence of SEQ ID NO: 23, and/or (ii) HVR-L1 comprising the amino acid sequence of SEQ ID NO: 24. In some embodiments, the immunoconjugate comprises an anti-CD79b antibody comprising at least one, at least two, or all three VH HVR sequences selected from (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23 and an HVR-L3 comprising the amino acid sequence of SEQ ID NO: 26. In some embodiments, the immunoconjugate comprises an anti-CD79b antibody that comprises an HVR-H3 comprising the amino acid sequence of SEQ ID NO: 23, an HVR-L3 comprising the amino acid sequence of SEQ ID NO: 26, and an HVR-H2 comprising the amino acid sequence of SEQ ID NO: 22. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23.

In some embodiments, the immunoconjugate comprises an anti-CD79b antibody comprising at least one, at least two, or all three VL HVR sequences selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises an HVR-L1 comprising the amino acid sequence of SEQ ID NO: 24 In some embodiments, the immunoconjugate comprises an anti-CD79b antibody that comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 21, (ii) HVR-H2 comprising the amino acid sequence of SEQ ID NO: 22, and (iii) HVR-H3 comprising an amino acid sequence selected from SEQ ID NO:23; and (b) a VL domain comprising at least one, at least two, or all three VL HVR sequences selected from (i) HVR-L1 comprising an amino acid sequence of SEQ ID NO: 24, (ii) HVR-L2 comprising the amino acid sequence of SEQ ID NO: 25, and (iii) HVR-L3 comprising the amino acid sequence of SEQ ID NO: 26. In some embodiments, the immunoconjugate comprises an anti-CD79b antibody that comprises at least one of: (i) HVR-H3 comprising the amino acid sequence of SEQ ID NO: 23, and/or (ii) HVR-L1 comprising the amino acid sequence of SEQ ID NO: 24.

In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26 (See, Table A). In some embodiments, the immunoconjugate comprises at least one of: HVR-H3 comprising the amino acid sequence of SEQ ID NO: 23 and/or HVR-L1 comprising an amino acid sequence of SEQ ID NO: 24 (See, Table A). In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26 (See, Table A).

TABLE A
HVR Amino Acid Sequences.

| | | SEQ ID |
|---|---|---|
| Name | Sequence | NO |
| huMA79bv28 HVR H1 | GYTESSYWIE | 21 |
| huMA79bv28 HVR H2 | GEILPGGGDTNYNEIFKG | 22 |
| huMA79bv28 HVR H3 | TRRVPIRLDY | 23 |
| huMA79bv28 HVR L1 | KASQSVDYEGDSFLN | 24 |
| huMA79bv28 HVR L2 | AASNLES | 25 |
| huMA79bv28 HVR L3 | QQSNEDPLT | 26 |

In some embodiments, the immunoconjugate comprises an anti-CD79b antibody that comprises, according to numbering in Kabat et al., the CDR-H, CDR-H2, CDR-H3, CDR-L1, CDR-L2, and CDR-L3 of the anti-CD79b antibody in polatuzumab vedotin-piiq.

In some embodiments, the anti-CD79b immunoconjugate comprises a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VL_{KI}) framework and/or the VH framework VH_{III}. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the immunoconjugate comprises an anti-CD79b antibody that comprises at least one, two, three, four, five, six, seven, or eight framework regions (FRs) selected from (a) a heavy chain FR (HC FR) 1 comprising the amino acid sequence of SEQ ID NO: 27; (b) an HC FR2 comprising the amino acid sequence of SEQ ID NO: 28; (c) an HC FR3 comprising the amino acid sequence of SEQ ID NO: 29; (d) an HC FR4 comprising an amino acid sequence of SEQ ID NO: 30; (e) a light chain FR (LC FR) 1 comprising the amino acid sequence of SEQ ID NO: 31; (f) an LC FR2 comprising the amino acid sequence of SEQ ID NO: 32; (g) an LC FR3 comprising the amino acid sequence of SEQ ID NO: 33; and (h) an LC FR4 comprising the amino acid sequence of SEQ ID NO: 34 (See, Table B).

In some embodiments, the immunoconjugate comprises an anti-CD79b antibody that comprises at least one, at least two, at least three, or all four HC FR sequences selected from (a) an HC FR1 comprising the amino acid sequence of SEQ ID NO: 27; (b) an HC FR2 comprising the amino acid sequence of SEQ ID NO: 28; (c) an HC FR3 comprising the amino acid sequence of SEQ ID NO: 29; and (d) an HC FR4 comprising an amino acid sequence of SEQ ID NO: 30. In some embodiments, the immunoconjugate comprises an anti-CD79b antibody that comprises at least one, at least two, at least three, or all four LC FR sequences selected from (a) an LC FR1 comprising the amino acid sequence of SEQ ID NO: 31; (b) an LC FR2 comprising the amino acid sequence of SEQ ID NO: 32; (c) an LC FR3 comprising the amino acid sequence of SEQ ID NO: 33; and (d) an LC FR4 comprising the amino acid sequence of SEQ ID NO: 34.

In some embodiments, the immunoconjugate comprises an anti-CD79b antibody that comprises (a) an HC FR 1 comprising the amino acid sequence of SEQ ID NO: 27; (b) an HC FR2 comprising the amino acid sequence of SEQ ID NO: 28; (c) an HC FR3 comprising the amino acid sequence of SEQ ID NO: 29; and (d) an HC FR4 comprising the amino acid sequence of SEQ ID NO: 30. In some embodiments, the immunoconjugate comprises an anti-CD79b antibody that comprises (a) an LC FR1 comprising the amino acid sequence of SEQ ID NO: 31; (b) an LC FR2 comprising the amino acid sequence of SEQ ID NO: 32; (c) an LC FR3 comprising the amino acid sequence of SEQ ID NO: 33; and (d) an LC FR4 comprising the amino acid sequence of SEQ ID NO: 34.

In some embodiments, the immunoconjugate comprises an anti-CD79b antibody that comprises (a) a VH domain comprising at least one, at least two, at least three, or all four HC FR sequences selected from (i) an HC FR1 comprising the amino acid sequence of SEQ ID NO: 27; (ii) an HC FR2 comprising the amino acid sequence of SEQ ID NO: 28; (iii) an HC FR3 comprising the amino acid sequence of SEQ ID NO: 29; and (iv) an HC FR4 comprising an amino acid sequence of SEQ ID NO: 30; and (b) a VL domain comprising at least one, at least two, at least three, or all four LC FR sequences selected from (i) an LC FR1 comprising the amino acid sequence of SEQ ID NO: 31; (ii) an LC FR2 comprising the amino acid sequence of SEQ ID NO: 32; (iii) an LC FR3 comprising the amino acid sequence of SEQ ID NO: 33; and (iv) an LC FR4 comprising the amino acid sequence of SEQ ID NO: 34.

In some embodiments, the immunoconjugate comprises an anti-CD79b antibody that comprises (a) an HC FR1 comprising the amino acid sequence of SEQ ID NO: 27; (b) an HVR-H1 comprising the amino acid sequence of SEQ ID NO: 21; (c) an HC FR2 comprising the amino acid sequence of SEQ ID NO: 28; (d) HVR-H2 comprising the amino acid sequence of SEQ ID NO: 22; (e) an HC FR3 comprising the amino acid sequence of SEQ ID NO: 29; (f) an HVR-H3 comprising the amino acid sequence of SEQ ID NO: 23; (g) an HC FR4 comprising the amino acid sequence of SEQ ID NO: 30; (h) an LC FR1 comprising the amino acid sequence of SEQ ID NO: 31; (i) an HVR-L1 comprising an amino acid sequence of SEQ ID NO: 24; (j) an LC FR2 comprising the amino acid sequence of SEQ ID NO: 32; (k) an HVR-L2 comprising the amino acid sequence of SEQ ID NO: 25; (1) an LC FR3 comprising the amino acid sequence of SEQ ID NO: 33; (m) an HVR-L3 comprising the amino acid sequence of SEQ ID NO: 26; and (n) an LC FR4 comprising the amino acid sequence of SEQ ID NO: 34.

TABLE B
Heavy Chain and Light Chain Framework
Region Amino Acid Sequences.

| | | SEQ |
|---|---|---|
| | | ID |
| Name | Sequence | NO |
| huMA79bv28 heavy | EVQLVESGGGLVQPGGSLRLS | 27 |
| chain (HC) | CAAS | |
| framework | | |
| region (FR) 1 | | |
| huMA79bv28 HC FR2 | WVRQAPGKGLEWI | 28 |
| huMA79bv28 HC FR3 | RATFSADTSKNTAYLQMNSLR | 29 |
| | AEDTAVYYC | |
| huMA79bv28 HC FR4 | WGQGTLVTVSS | 30 |
| huMA79bv28 light | DIQLTQSPSSLSASVGDRVTI | 31 |
| chain (LC) FR1 | TC | |
| huMA79bv28 LC FR2 | WYQQKPGKAPKLLTY | 32 |
| huMA79bv28 LC FR3 | GVPSRFSGSGSGTDFTLTISS | 33 |
| | LQPEDFATYYC | |
| huMA79bv28 LC FR4 | FGQGTKVEIKR | 34 |

In some embodiments, the immunoconjugate (e.g., the anti-CD79b immunoconjugate) comprises an anti-CD79b antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In some embodiments, a VH sequence having at least 90%, 91%, 92%, 93%, 94%, 95%, 96%, 97%, 98%, or 99% identity to the amino acid sequence of SEQ ID NO: 19 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 19. In some embodiments, a total of 1 to 5 amino acids have been substituted, inserted and/or deleted in SEQ ID NO: 19. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 19, including post-translational modifications of that sequence. In some embodiments, the VH comprises one, two or three HVRs selected from: (a) HVR-H1 comprising the amino acid sequence of SEQ ID NO: 21, (b) HVR-H2 comprising the amino acid sequence of SEQ ID NO: 22, and (c) HVR-H3 comprising the amino acid sequence of SEQ ID NO: 17 or SEQ ID NO: 23.

(SEQ ID NO: 19)
EVQLVESGGG LVQPGGSLRL SCAASGYTFS SYWIEWVRQA
PGKGLEWIGE ILPGGGDTNY NEIFKGRATF SADTSKNTAY
LQMNSLRAED TAVYYCTRRV PIRLDYWGQG TLVTVSS

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity to the amino acid sequence of SEQ ID NO: 20 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 20. In certain embodiments, a total of 1 to 5 amino acids have been substituted, inserted and/or deleted in SEQ ID NO: 20. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 20, including post-translational modifications of that sequence. In some embodiments, the VL comprises one, two or three HVRs selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

(SEQ ID NO: 20)
DIQLTQSPSS LSASVGDRVT ITCKASQSVD YEGDSFLNWY
QQKPGKAPKL LIYAASNLES GVPSRFSGSG SGTDFTLTIS
SLQPEDFATY YCQQSNEDPL TFGQGTKVEI KR

In some embodiments, the immunoconjugate (e.g., the anti-CD79b immunoconjugate) comprises an anti-CD79b antibody that comprises VH as in any of the embodiments provided herein, and a VL as in any of the embodiments provided herein. In some embodiments, the immunoconjugate comprises an anti-CD79b antibody that comprises the VH and VL sequences in SEQ ID NO: 19 and SEQ ID NO: 20, respectively, including post-translational modifications of those sequences.

In some embodiments, the immunoconjugate comprises an anti-CD79b antibody that comprises the VH and the VL of the anti-CD79b antibody in polatuzumab vedotin-piiq.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 19 and a VL sequence of SEQ ID NO: 20.

In some embodiments, the immunoconjugate comprises an anti-CD79b antibody that is a monoclonal antibody, a chimeric antibody, humanized antibody, or human antibody. In some embodiments, the immunoconjugate comprises an antigen-binding fragment of an anti-CD79b antibody described herein, e.g., a Fv, Fab, Fab′, scFv, diabody, or F(ab′)₂ fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody that comprises a heavy chain comprising the amino acid sequence of SEQ ID NO: 36, and/or a light chain comprising the amino acid sequence of SEQ ID NO: 35. In some embodiments, the immunoconjugate comprises an anti-CD79b antibody that comprises a heavy chain comprising the amino acid sequence of SEQ ID NO: 37 and/or a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and/or a light chain comprising the amino acid sequence of SEQ ID NO: 38. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38.

In some embodiments, the immunoconjugate comprises an anti-CD79b antibody that comprises the heavy chain and the light chain of the anti-CD79b antibody in polatuzumab vedotin-piiq.

In some embodiments, the immunoconjugate comprises an anti-CD79b antibody that comprises the heavy chain and the light chain of the anti-CD79b antibody in iladatuzumab vedotin. In some embodiments, the anti-CD79b antibody comprises a heavy chain comprising the amino acid sequence of SEQ ID NO: 37 and a light chain comprising the amino acid sequence of SEQ ID NO: 35.

C. Drugs/Cytotoxic Agents

Anti-CD79 immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. That is, the anti-CD79 immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79 immunoconjugates used in the methods provided herein include those with anticancer activity. In some embodiments, the anti-CD79 immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of the anti-CD79 immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

(i) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see, e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamitocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/−dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9-SH (U.S. Pat. No. 4,424,219) (prepared, for example, by the reaction of maytansinol with H₂S or P₂S₅); C-14-alkoxymethyl (demethoxy/CH₂ OR)(U.S. Pat. No. 4,331,598); C-14-hydroxymethyl or acyloxymethyl (CH₂OH or CH₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

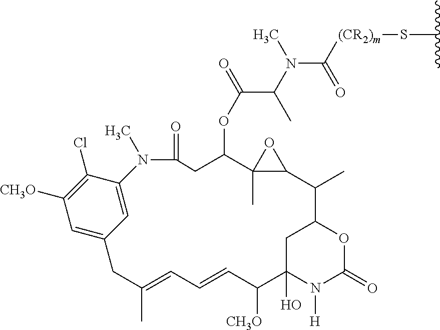

wherein the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. No. 5,208,020; Chari et al (1992) Cancer Res. 52:127-131; Liu et al (1996) Proc. Nat. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; 633,410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

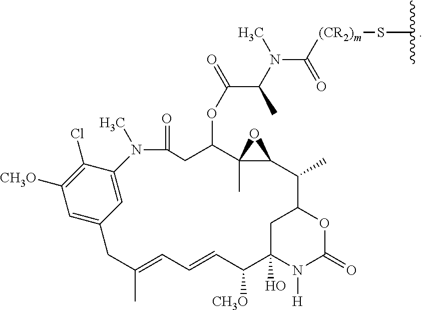

Exemplary embodiments of maytansinoid drug moieties include, but are not limited to, DM1; DM3; and DM4, having the structures:

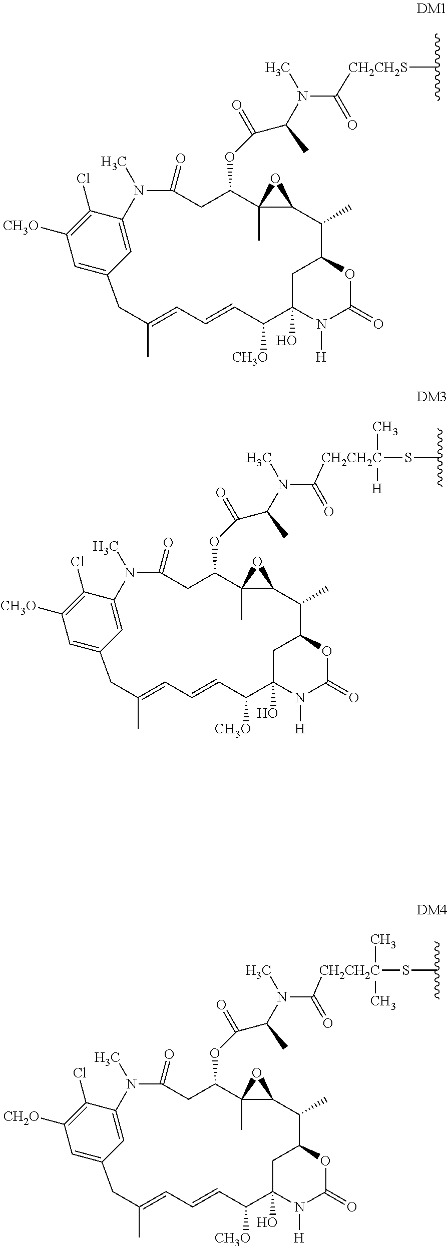

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

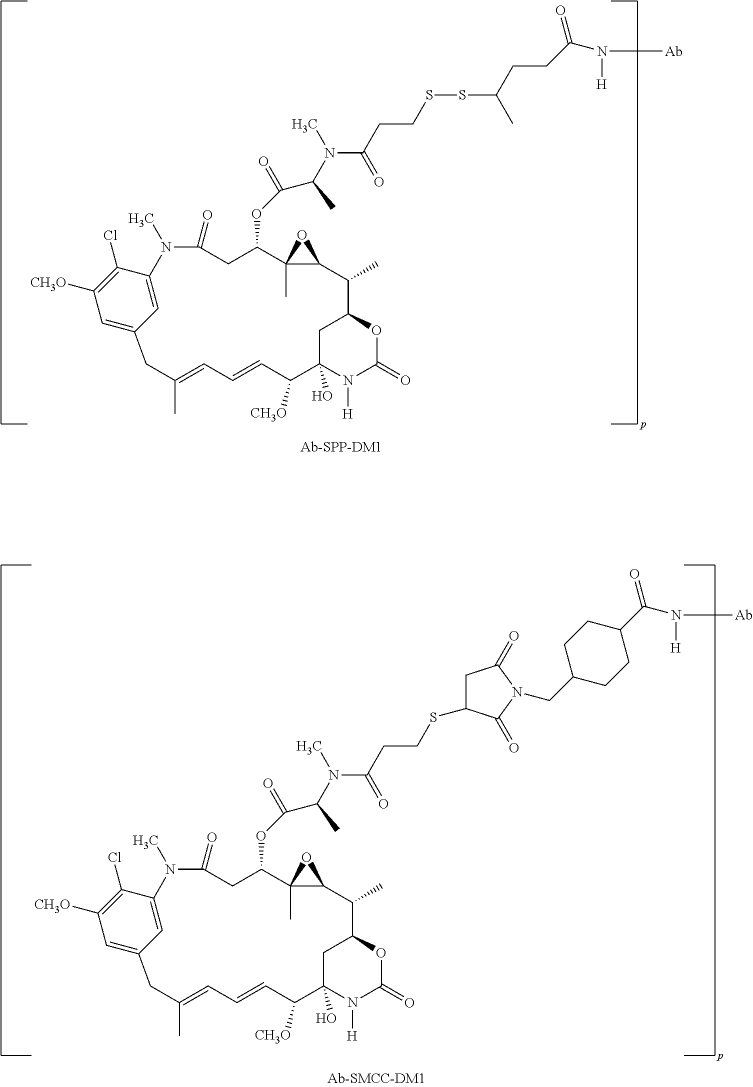

Exemplary antibody-drug conjugates where DM1 is linked through a BMPEO linker to a thiol group of the antibody have the structure and abbreviation:

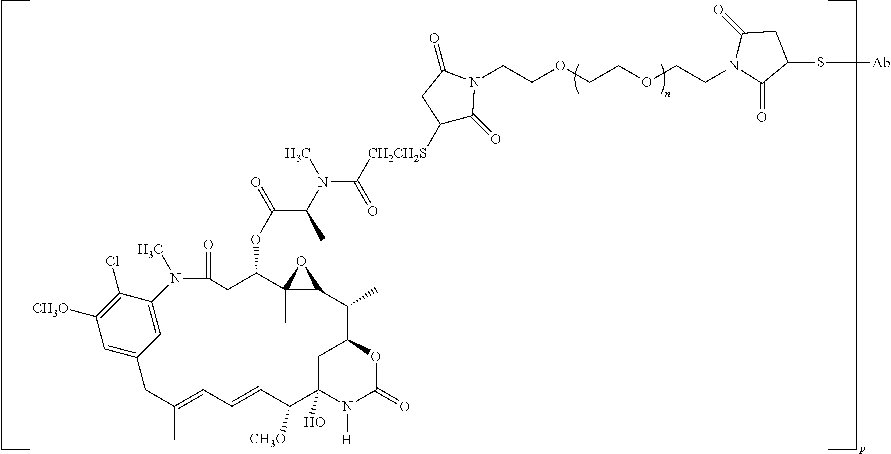

wherein Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Nat. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

(2) Auristatins and dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

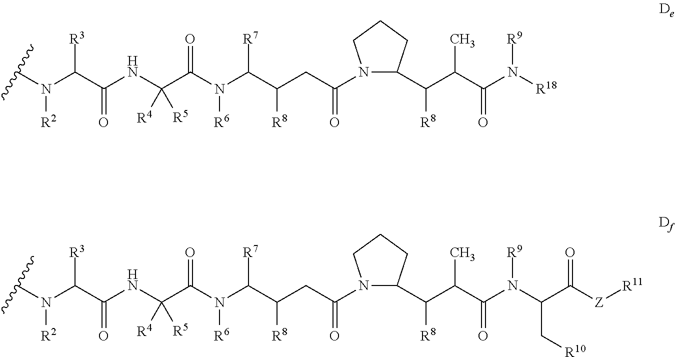

wherein the wavy line of D_{E} and D_{F} indicates the covalent attachment site to an antibody or antibody-linker component, and independently at each location:

R² is selected from H and C₁-C₈ alkyl;

R³ is selected from H, C₁—C alkyl, C₃—C carbocycle, aryl, C₁—C alkyl-aryl, C₁-C₈ alkyl-(C₃-C₈ carbocycle), C₃-C₈ heterocycle and C₁-C₈ alkyl-(C₃-C₈ heterocycle);

R⁴ is selected from H, C₁—C alkyl, C₃—C carbocycle, aryl, C₁—C alkyl-aryl, C₁-C₈ alkyl-(C₃-C₈ carbocycle), C₃-C₈ heterocycle and C₁-C₈ alkyl-(C₃-C₈ heterocycle);

R⁵ is selected from H and methyl;

or R⁴ and R⁵ jointly form a carbocyclic ring and have the formula —(CR^{a}R^{b})ₙ— wherein Ra and R^{b} are independently selected from H, C₁-C₈ alkyl and C₃-C₈ carbocycle and n is selected from 2, 3, 4, 5 and 6;

R⁶ is selected from H and C₁-C₈ alkyl;

R⁷ is selected from H, C₁—C alkyl, C₃—C carbocycle, aryl, C₁—C alkyl-aryl, C₁-C₈ alkyl-(C₃-C₈ carbocycle), C₃-C₈ heterocycle and C₁-C₈ alkyl-(C₃-C₈ heterocycle);

each R⁸ is independently selected from H, OH, C₁—C alkyl, C₃—C carbocycle and O—(C₁-C₈ alkyl);

R⁹ is selected from H and C₁-C₈ alkyl;

R¹⁰ is selected from aryl or C₃-C₈ heterocycle;

Z is O, S, NH, or NR¹², wherein R¹² is C₁-C₈ alkyl;

R¹¹ is selected from H, C₁-C₂₀ alkyl, aryl, C₃—C heterocycle, —(R¹³O)ₘ—R¹⁴, or —(R¹³O)ₘ—CH(R¹⁵)₂;

m is an integer ranging from 1-1000;

R¹³ is C₂-C₈ alkyl;

R¹⁴ is H or C₁-C₈ alkyl;

each occurrence of R¹⁵ is independently H, COOH, —(CH₂)—N(R¹⁶)₂, —(CH₂)—SO₃H, or —(CH₂)—SO₃—C₁-C₈ alkyl;

each occurrence of R¹⁶ is independently H, C₁-C₈ alkyl, or —(CH₂)—COOH;

R¹⁸ is selected from —C(R)₂—C(R)₂-aryl, —C(R⁸)₂—C(R⁸)₂—(C₃-C₈ heterocycle), and —C(R⁸)₂—C(R⁸)₂—(C₃-C₈ carbocycle); and

n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)n-N(R¹⁶)₂, and R¹⁶ is —C₁-C₈ alkyl or —(CH₂)n-COOH.

In another embodiment, when Z is —NH, R¹¹ is —CH(R¹⁵)₂, wherein R¹⁵ is —(CH₂)—SO₃H.

An exemplary auristatin embodiment of formula D_{E} is MMAE, wherein the wavy line indicates the covalent attachment to a linker (L) of an anti-CD79b immunoconjugate:

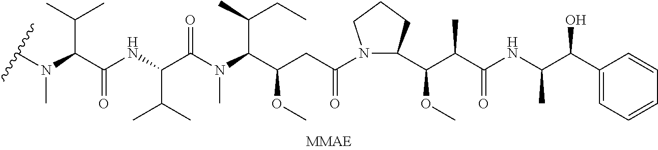 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an anti-CD79b immunoconjugate:

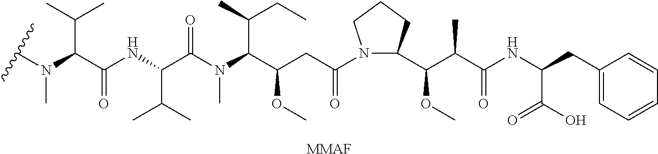

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

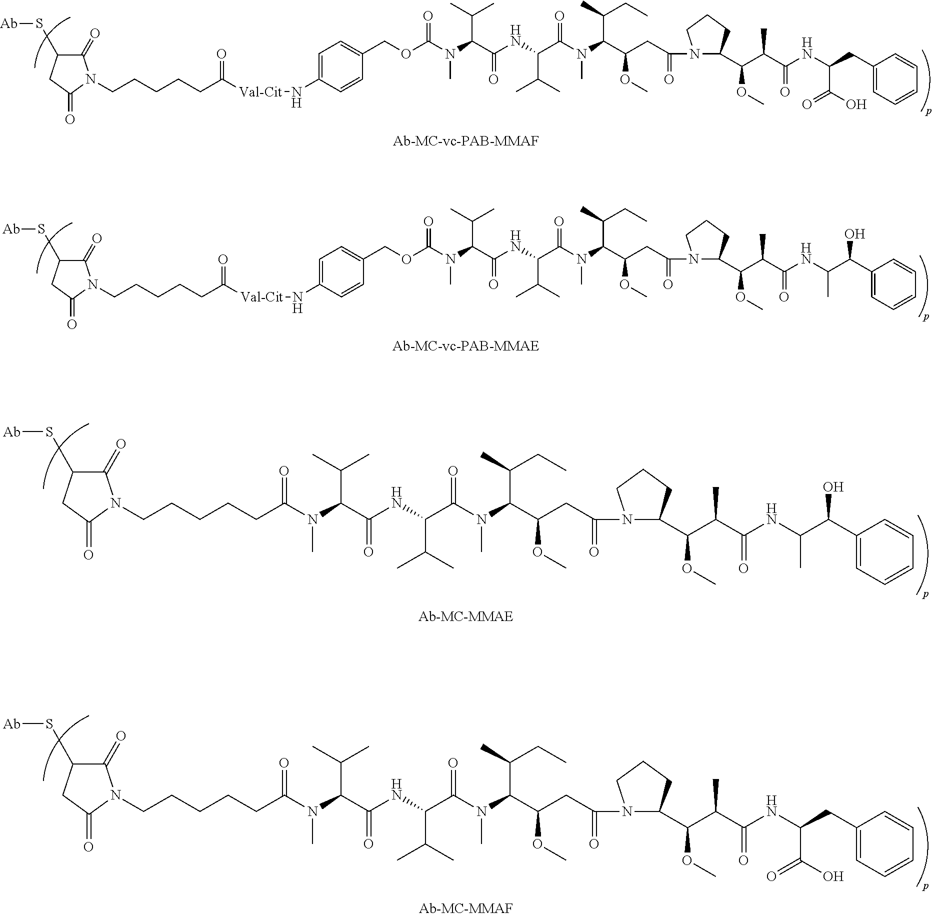

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of Ab-MC-vc-PAB-MMAE, wherein p is, e.g., about 1 to about 8; about 2 to about 7; about 3 to about 5; about 3 to about 4; or about 3.5, wherein the anti-CD79b antibody (Ab) comprises a heavy chain comprising the amino acid sequence of SEQ ID NO: 36, and a light chain comprising the amino acid sequence of SEQ ID NO: 35. In some embodiments, the anti-CD79b immunoconjugate is polatuzumab vedotin-piiq. Polatuzumab vedotin-piiq has the IUPHAR/BPS Number 8404, the KEGG Number D10761, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Nonlimiting exemplary embodiments of anti-CD79b immunoconjugates of Formula I comprising MMAE and various linker components further include Ab-MC-PAB-MMAE and Ab-PAB-MMAE.

Typically, peptide-based drug moieties can be prepared by forming a peptide bond between two or more amino acids and/or peptide fragments. Such peptide bonds can be prepared, for example, according to a liquid phase synthesis method (see, e.g., E. Schröder and K. Lübke, “The Peptides”, volume 1, pp 76-136, 1965, Academic Press). Auristatin/dolastatin drug moieties may, in some embodiments, be prepared according to the methods of: U.S. Pat. Nos. 7,498,298; 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

In some embodiments, auristatin/dolastatin drug moieties of formulas D_{E} such as MMAE, and D_{F}, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784 and then conjugated to an antibody of interest.

(3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

(4) Other Drug Moieties

In some embodiments, an anti-CD79b immunoconjugate comprises geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹ I¹²⁵ Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³ Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as 0-galactosidase and neuraminidase, which are useful for converting glycosylated prodrugs into free drugs; P-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature 312:604-608 (1984).

D. Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p >5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6; from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anti-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

E. Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i)N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

### V. Anti-CD20 Agents

Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table C.

TABLE C
Properties of type I and type 11 anti-CD20 antibodies

| Type I anti-CD20 antibodies | Type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type I anti-CD20 antibodies include e.g., rituximab, HI47 IgG3 (ECACC, hybridoma), 2C6 IgG1 (as disclosed in WO 2005/103081), 2F² IgG1 (as disclosed and WO 2004/035607 and WO 2005/103081) and 2H7 IgG1 (as disclosed in WO 2004/056312).

In some embodiments, the anti-CD20 antibody used a method of treatment provided herein is rituximab. In some embodiments, the rituximab (reference antibody; example of a type I anti-CD20 antibody) is a genetically engineered chimeric human gamma 1 murine constant domain containing monoclonal antibody directed against the human CD20 antigen. However, this antibody is not glycoengineered and not afucosylated and thus has an amount of fucose of at least 85%. This chimeric antibody comprises human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with diffuse large B-cell lymphoma (DLBCL), relapsed or refractory low-grade or follicular, CD20 positive, B-cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC).

In some embodiments, the anti-CD20 antibody used in a method of treatment provided herein comprises, according to numbering in Kabat et al., the CDR-H1, CDR-H2, CDR-H3, CDR-L1, CDR-L2, and CDR-L3 of rituximab. In some embodiments, anti-CD20 antibody used in a method of treatment provided herein comprises the VH and the VL of rituximab. In some embodiments, anti-CD20 antibody used in a method of treatment provided herein comprises the heavy chain and the light chain of rituximab. As used herein, the term “rituximab” refers to an anti-CD20 antibody having the CAS Registry Number 174722-31-7.

In some embodiments, the anti-CD20 antibody used in a method of treatment provided herein is an afucosylated anti-CD20 antibody.

Examples of type II anti-CD20 antibodies include e.g., humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype. In some embodiments, the type II anti-CD20 antibody, e.g., a GA101 antibody, has increased antibody dependent cellular cytotoxicity (ADCC). In some embodiments, the type II anti-CD20 antibodies, more preferably an afucosylated humanized B-Ly1 antibody as described in WO 2005/044859 and WO 2007/031875.

In some embodiments, the anti-CD20 antibody used in a method of treatment provided herein is GA101 antibody. In some embodiments, the GA101 antibody as used herein refers to any one of the following antibodies that bind human CD20: (1) an antibody comprising an HVR-H1 comprising the amino acid sequence of SEQ ID NO:5, an HVR-H2 comprising the amino acid sequence of SEQ ID NO:6, an HVR-H3 comprising the amino acid sequence of SEQ ID NO:7, an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10; (2) an antibody comprising a VH domain comprising the amino acid sequence of SEQ ID NO:11 and a VL domain comprising the amino acid sequence of SEQ ID NO:12, (3) an antibody comprising an amino acid sequence of SEQ ID NO:13 and an amino acid sequence of SEQ ID NO: 14; (4) an antibody known as obinutuzumab, or (5) an antibody that comprises an amino acid sequence that has at least 95%, 96%, 97%, 98% or 99% sequence identity with amino acid sequence of SEQ ID NO:13 and that comprises an amino acid sequence that has at least 95%, 96%, 97%, 98% or 99% sequence identity with an amino acid sequence of SEQ ID NO: 14. In one embodiment, the GA101 antibody is an IgG1 isotype antibody.

In some embodiments, the anti-CD20 antibody used in a method of treatment provided herein is a humanized B-Ly1 antibody. In some embodiments, the humanized B-Ly1 antibody refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 3; variable region of the murine light chain (VL): SEQ ID NO: 4-see Poppema, S. and Visser, L., Biotest Bulletin 3 (1987) 131-139) by chimerization with a human constant domain from IgG1 and following humanization (see WO 2005/044859 and WO 2007/031875). The humanized B-Ly1 antibodies are disclosed in detail in WO 2005/044859 and WO 2007/031875.

In some embodiments, the humanized B-Ly1 antibody has variable region of the heavy chain (VH) selected from group of SEQ ID NO:15-16 and 40-55 (corresponding to B-HH2 to B-HH9 and B-HL8 to B-HL17 of WO 2005/044859 and WO 2007/031875). In some embodiments, the variable domain is selected from the group consisting of SEQ ID NO: 15, 16, 42, 44, 46, 48 and 50 (corresponding to B-HH2, BHH-3, B-HH6, B-HH8, B-HL8, B-HL11 and B-HL13 of WO 2005/044859 and WO 2007/031875). In some embodiments, the humanized B-Ly1 antibody has variable region of the light chain (VL) of SEQ ID NO:55 (corresponding to B-KV1 of WO 2005/044859 and WO 2007/031875). In some embodiments, the humanized B-Ly1 antibody has a variable region of the heavy chain (VH) of SEQ ID NO:42 (corresponding to B-HH6 of WO 2005/044859 and WO 2007/031875) and a variable region of the light chain (VL) of SEQ ID NO:55 (corresponding to B-KV1 of WO 2005/044859 and WO 2007/031875). In some embodiments, the humanized B-Ly1 antibody is an IgG1 antibody. Such afucosylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In some embodiments, the afucosylated glyco-engineered humanized B-Ly1 is B-HH6-B-KV1 GE. In some embodiments, the anti-CD20 antibody is obinutuzumab (recommended INN, WHO Drug Information, Vol. 26, No. 4, 2012, p. 453). As used herein, obinutuzumab is synonymous for GA101 or RO5072759. It is commercially available for therapeutic use under the trade name GAZYVA®, and is provided as a 1000 mg/40 mL (25 mg/mL) single-dose vial. This replaces all previous versions (e.g., Vol. 25, No. 1, 2011, p.75-76), and is formerly known as afutuzumab (recommended INN, WHO Drug Information, Vol. 23, No. 2, 2009, p. 176; Vol. 22, No. 2, 2008, p. 124). In some embodiments, the humanized B-Ly1 antibody is an antibody comprising a heavy chain comprising the amino acid sequence of SEQ ID NO:17 and a light chain comprising the amino acid sequence of SEQ ID NO:18, or an antigen-binding fragment thereof such antibody. In some embodiments, the humanized B-Ly1 antibody comprises a heavy chain variable region comprising the three heavy chain CDRs of SEQ ID NO:17 and a light chain variable region comprising the three light chain CDRs of SEQ ID NO:18.

In some embodiments, the anti-CD20 antibody used in a method of treatment provided herein comprises, according to numbering in Kabat et al., the CDR-H1, CDR-H2, CDR-H3, CDR-L1, CDR-L2, and CDR-L3 of obinutuzumab. In some embodiments, anti-CD20 antibody used in a method of treatment provided herein comprises the VH and the VL of obinutuzumab. In some embodiments, anti-CD20 antibody used in a method of treatment provided herein comprises the heavy chain and the light chain of obinutuzumab.

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. In some embodiments, the amount of fucose is about 60% or less of the total amount of oligosaccharides at Asn297 (in one embodiment the amount of fucose is between about 40% and about 60%, in another embodiment the amount of fucose is about 50% or less, and in still another embodiment the amount of fucose is about 30% or less). In some embodiments, the oligosaccharides of the Fc region are bisected. These glycoengineered humanized B-Ly1 antibodies have an increased ADCC.

The “ratio of the binding capacities to CD20 on Raji cells (ATCC-No. CCL-86) of an anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), as described in Example No. 2, and calculated as follows:

$$Ratio of the binding capacities to CD 20 on Raji cells ( ATCC \text{-} No . CCL \text{-} 86 ) = \frac{MFI ( Cy 5 \text{-} anti \text{-} CD 20 antibody )}{MFI ( Cy 5 \text{-} rituximab )} \times \frac{Cy 5 \text{-} labeling ratio ( Cy 5 \text{-} rituximab )}{Cy 5 \text{-} labeling ratio ( Cy 5 \text{-} anti \text{-} CD 20 antibody )}$$

MFI is the mean fluorescent intensity. The “Cy5-labeling ratio” as used herein means the number of Cy5-label molecules per molecule antibody.

Typically said type II anti-CD20 antibody has a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

By “antibody having increased antibody dependent cellular cytotoxicity (ADCC)”, it is meant an antibody, as that term is defined herein, having increased ADCC as determined by any suitable method known to those of ordinary skill in the art.

An exemplary accepted in vitro ADCC assay is described below:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

In some embodiments, the “increased ADCC” can be obtained by, for example, mutating and/or glycoengineering of said antibodies. In some embodiments, the anti-CD20 antibody is glycoengineered to have a biantennary oligosaccharide attached to the Fc region of the antibody that is bisected by GlcNAc. In some embodiments, the anti-CD20 antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down). In some embodiments, the anti-CD20 antibody sequence has been engineered in its Fc region to enhance ADCC. In some embodiments, such engineered anti-CD20 antibody variant comprises an Fc region with one or more amino acid substitutions at positions 298, 333, and/or 334 of the Fc region (EU numbering of residues)).

In some embodiments, the term “complement-dependent cytotoxicity (CDC)” refers to lysis of human cancer target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In some embodiments, the assay is performed with ⁵¹Cr or Eu labeled tumor cells and measurement of released ⁵¹Cr or Eu. Controls include the incubation of the tumor target cells with complement but without the antibody.

In some embodiments, the anti-CD20 antibody is a monoclonal antibody, e.g., a human antibody. In some embodiments, the anti-CD20 antibody is an antibody fragment, e.g., a Fv, Fab, Fab′, scFv, diabody, or F(ab′)₂ fragment. In some embodiments, the anti-CD20 antibody is a substantially full length antibody, e.g., an IgG1 antibody, IgG2a antibody or other antibody class or isotype as defined herein.

In some embodiments, the anti-CD20 antibody is any of ABP 798 (Amgen, USA), Zytux (AryoGen Pharmed, Iran), AcellBia/Usmal (Biocad, Russia), BI 695500 (Boehringer Ingelheim, Germany), Truxima (Celltrion, South Korea), Blitzima (Celltrion, South Korea), Ritemvia (Celltrion, South Korea), Rituzena/Tuxella (Celltrion, South Korea), CT-P10 (Celltrion, South Korea), Reditux (Dr Reddy's Laboratories, India), Maball (Hetero Group, India), MabTas (Intas Biopharmaceuticals, India), JHL1101 (JHL Biotech, Taiwan), Novex (RTXM83) (mAbxience/Laboratorio Elea, Spain/Argentina), MabionCD20 (Mabion, Poland; Mylan, India), PF-05280586 (Pfizer, USA), Kikuzubam (Probiomed, Mexico), Rituximab (Zenotech Laboratories), RituxiRel (Reliance Life Sciences, India), SAIT101 (Samsung BioLogics, South Korea), Rixathon/Riximyo (GP2013) (Sandoz, Switzerland), HLX01 (Shanghai Henlius Biotech, China), TLO11 (Teva Pharmaceutical Industries, Israel; Lonza, Switzerland), or Redditux (TRPharma, Turkey).

### VI. Antibodies

In some embodiments, an antibody (e.g., an anti-CD79b antibody or an anti-CD20 antibody) used in a method of treatment provided herein may incorporate any of the features, singly or in combination, as described in below.

A. Antibody Affinity

In certain embodiments, an antibody (e.g., an anti-CD79b antibody or an anti-CD20 antibody) used in a method of treatment provided herein has a dissociation constant (Kd) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g., 10⁻⁸ M or less, e.g., from 10⁻⁸ M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [I²]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE©-2000 or a BIACORE ©-3000 (BIAcore, Inc., Piscataway, N.J.) at 25° C. with immobilized antigen CM5 chips at -10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20TM) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

B. Antibody Fragments

In certain embodiments, an antibody (e.g., an anti-CD79b antibody or an anti-CD20 antibody) used in a method of treatment provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

C. Chimeric and Humanized Antibodies

In certain embodiments, an antibody a (e.g., an anti-CD79b antibody or an anti-CD20 antibody) used in a method of treatment provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

D. Human Antibodies

In certain embodiments, an antibody (e.g., an anti-CD79b antibody or an anti-CD20 antibody) used in a method of treatment provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

E. Library-Derived Antibodies

In some embodiments, an antibody (e.g., an anti-CD79b antibody or an anti-CD20 antibody) used in a method of treatment provided herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

F. Multispecific Antibodies

In certain embodiments, an antibody (e.g., an anti-CD79b antibody or an anti-CD20 antibody) used in a method of treatment provided herein is a multispecific antibody, e.g., a bispecific antibody. Multispecific antibodies are monoclonal antibodies that have binding specificities for at least two different sites. In certain embodiments, one of the binding specificities is for one antigen (e.g., CD79b or CD20) and the other is for any other antigen. In certain embodiments, one of the binding specificities is for one antigen (e.g., CD79b or CD20) and the other is for CD3. See, e.g., U.S. Pat. No. 5,821,337. In certain embodiments, bispecific antibodies may bind to two different epitopes of a single antigen (e.g., CD79b or CD20). Bispecific antibodies may also be used to localize cytotoxic agents to cells which express the antigen (e.g., CD79b or CD20). Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Nat. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to CD79b as well as another, different antigen (see, US 2008/0069820, for example).

G. Antibody Variants

In certain embodiments, amino acid sequence variants of an antibody (e.g., an anti-CD79b antibody or an anti-CD20 antibody) used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

(i) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table D under the heading of “preferred substitutions.” More substantial changes are provided in Table D under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE D

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

(ii) Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96© non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

(iv) Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. See, e.g., WO 2009/012268, for exemplary cysteine engineered anti-CD79b antibodies for use in the methods described herein. Cysteine engineered antibodies may be generated as described, e.g., in U.S. Pat. No. 7,521,541.

(v) Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Nat. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

H. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

I. Assays

An antibody (e.g., an anti-CD79b antibody or an anti-CD20 antibody) used in a method of treatment provided herein may be identified, screened for, or characterized for their physical/chemical properties and/or biological activities by various assays known in the art.

In one aspect, an antibody (e.g., an anti-CD79b antibody or an anti-CD20 antibody) used in a method of treatment provided herein is tested for its antigen binding activity, e.g., by known methods such as ELISA, BIACore®, FACS, or Western blot.

In another aspect, competition assays may be used to identify an antibody that competes with any of the antibodies described herein for binding to the target antigen. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized antigen is incubated in a solution comprising a first labeled antibody that binds to antigen (e.g., any of the antibodies described herein) and a second unlabeled antibody that is being tested for its ability to compete with the first antibody for binding to antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch. 14 (Cold Spring Harbor Laboratory, Cold Spring Harbor, N.Y.).

### VII. Chemotherapeutic Agents

In some embodiments, the one or more chemotherapeutic agents comprise chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, everolimus, sotrataurin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantronesom; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional examples include of chemotherapeutic agents include bendamustine (or bendamustine-HCl) (TREANDA®), ibrutinib, lenalidomide, and/or idelalisib (GS-1101).

In some embodiments, the one or more chemotherapeutic agents comprise anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine.

In some embodiments, the one or more chemotherapeutic agents comprise a topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

In some embodiments, the one or more chemotherapeutic agents comprise antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ublituximab, ofatumumab, ibritumomab tiuxetan, pertuzumab (OMNITARG®, 2C4, Genentech), trastuzumab (HERCEPTIN, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

In some embodiments, the one or more chemotherapeutic agents comprise alkylating agents. Alkylating agents are a class of antineoplastic or anticancer drugs which act by inhibiting the transcription of DNA into RNA and thereby stopping the protein synthesis. Alkylating agents substitute alkyl groups (CₙH2ₙ₊₁) for hydrogen atoms on DNA, resulting in the formation of cross links within the DNA chain, thereby causing DNA strand breaks, which lead to abnormal base pairing, inhibition of cell division, and, eventually, cell death. This action occurs in all cells, but rapidly dividing cells, such as cancer cells, are typically most sensitive to the effects of alkylating agents

Alkylating agents are generally separated into six classes: (1) nitrogen mustards which include, without limitation, e.g., mechlorethamine, cyclophosphamide, ifosfamide, bendamustine, melphalan and chlorambucil; (2) ethylenamine and methylenamine derivatives which include, without limitation, e.g., altretamine and thiotepa; (3) alkyl sulfonates which include, without limitation, e.g., busulfan; (4) nitrosoureas which include, without limitation, e.g., carmustine and lomustine; (5) triazenes which include, without limitation, e.g., dacarbazine and procarbazine, temozolomide; and (6) platinum-containing antineoplastic agents, which include, without limitation, e.g., cisplatin, carboplatin, and oxaliplatin. Any known alkylating agent (including, but not limited to those listed above) can be used in a method of treatment provided herein. Bendamustine is an exemplary alkylating agent used in the methods described herein. The chemical name for bendamustine is 4-(5-(Bis(2-chloroethyl)amino)-1-methyl-H-benzo[d]imidazol-2-yl)butanoic acid, and has the molecular formula of C₁₆H₂₁Cl₂N₃O₂ and a molecular weight of 358.263 g/mol. Bendamustine (CAS Registry #16506-27-7) is a bifunctional mechlorethamine derivative that contains a purine-like benzimidazole ring. Bendamustine is available as powder for solution and solution dosage forms. In some embodiments, the alkylating agent used in the methods described herein is a salt or solvate of bendamustine. In some embodiments, the bendamustine salt is bendamustine-HCl (CAS #3543-75-7), which has the molecular formula of C₁₆H2₁Cl₂N₃O₂.HCl and a molecular weight of 394.72 g/mol. Bendamustine-HCl is commercially available as BENDEKA, TREANDA, TREAKISYM, RIBOMUSTIN, LEVACT, MUSTIN, and others.

In some embodiments, the one or more chemotherapeutic agents comprise gemcitabine (e.g., GEMZAR®). Gemcitabine is an antimetabolite nucleoside analogue (2′,2′-difluorodeoxycytidine). As only its di- and tri-phosphate forms possess cytotoxic activity, it becomes active after intracellular phosphorylation by deoxycytidine kinase. Specifically, the triphosphate form competes with deoxycytidine triphosphate for incorporation into DNA as an inactive base, and the diphosphate form inhibits ribonucleotide reductase, an enzyme that is essential for normal DNA synthesis. Gemcitabine can also be referred to as “2′-deoxy-2′,2′-difluorocytidine monohydrochloride” (0-isomer) in accordance with IUPAC nomenclature and has the following structure:

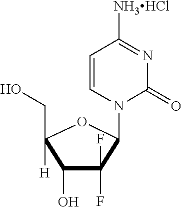

Use of the terms “2′-deoxy-2′,2′-difluorocytidine monohydrochloride (0-isomer)” or “gemcitabine” encompass (unless otherwise indicated) pharmaceutically acceptable solvates (including hydrates) and polymorphic forms or pharmaceutically acceptable salts thereof. Pharmaceutical compositions of 2′-deoxy-2′,2′-difluorocytidine monohydrochloride (0-isomer) can include one or more diluents, vehicles and/or excipients. One example of a pharmaceutical composition comprising 2′-deoxy-2′,2′-difluorocytidine monohydrochloride (β-isomer) is GEMZAR® (gemcitabine HCl). GEMZAR® comprises 2′-deoxy-2′,2′-difluorocytidine monohydrochloride (β-isomer) as the active ingredient and other inactive ingredients in a sterile form for intravenous use only. Vials of GEMZAR® contain either 200 mg or 1 g of gemcitabine HCl (expressed as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

In some embodiments, the one or more chemotherapeutic agents comprise oxaliplatin (e.g., ELOXATIN®). Oxaliplatin is a chemotherapeutic agent with the molecular formula C₈H₁₄N₂O₄Pt and the chemical name of cis-[(1R,2R)-1,2-cyclohexanediamine-N,N][oxalato(2-)-0,0] platinum. Its chemical structure is shown below:

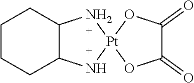

Use of the terms “cis-[(1R,2R)-1,2-cyclohexanediamine-N,N][oxalato(2-)-O,O] platinum” or “oxaliplatin” encompass (unless otherwise indicated) pharmaceutically acceptable solvates (including hydrates) and polymorphic forms or pharmaceutically acceptable salts thereof. The platinum atom of oxaliplatin forms 1,2-intrastrand crosslinks between two adjacent guanosine residues bending the double helix by approximately 30 degrees toward the major groove. Oxaliplatin has a non-hydrolyzable diaminocyclohexane (DACH) carrier ligand that is maintained in the final cytotoxic metabolites of the drug. Its reaction with DNA and other macromolecules proceeds by hydrolysis of one or both carboxylester groups of oxalate leaving a DACH platinum monoadduct or a bifuctional DACH-platinum crosslink. The intrinsic chemical and steric characteristics of the DACH-platinum adducts appear to contribute to the lack of cross-resistance with cisplatin (reviewed in Di Francesco et al., (2002) Cell Mol Life Sci, 59(11):1914-27). Alkaline hydrolysis of oxaliplatin gives the oxalato monodentate complex (pKa 7.23) and the dihydrated oxaliplatin complex in two consecutive steps. The monodentate intermediate is assumed to rapidly react with endogenous compounds (Jerremalm et al., (2003) J Pharm Sci, 92(2):436-438). The crystal structures of oxaliplatin bound to a DNA dodecamer duplex with the sequence 5′-d(CCTCTGGTCTCC) has been reported; the platinum atom forms a 1,2-intrastrand cross-link between two adjacent guanosine residues bending the double helix by approximately 30 degrees towards the major groove. The crystallography provided structural evidence for the importance of chirality in mediating the interaction between oxaliplatin and duplex DNA (Spingler et al., (2001) Inorg Chem, 40(22):5596-602). Thus, the success of oxaliplatin lies in its ability to induce DNA damage resulting from bulky adducts as well as intra- and inter-strand crosslinks (Takahara et al., (1995) Nature, 377(6550):649-52), as well as its ability to induce apoptosis (Boulikas and Vougiouka, (2003) Oncol Rep, 10(6):1663-82).

In some embodiments, the one or more chemotherapeutic agents comprise gemcitabine and oxaliplatin (e.g., GEMZAR® and ELOXATIN®). In some embodiments, the one or more chemotherapeutic agents are gemcitabine and oxaliplatin (e.g., GEMZAR® and ELOXATIN®).

### VIII. Pharmaceutical Formulations

Pharmaceutical formulations of any of the agents described herein (e.g., anti-CD79b immunoconjugates, anti-CD20 agents, and one or more chemotherapeutic agents) for use in any of the methods as described herein are prepared by mixing such agent(s) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

Additional details regarding pharmaceutical formulations comprising an anti-CD79 immunoconjugate are provided in WO 2009/099728 the contents of which are expressly incorporated by reference herein in their entirety.

### IX. Kits and Articles of Manufacture

In another embodiment, an article of manufacture or a kit is provided comprising an anti-CD79b immunoconjugate (such as described herein) and at least one additional agent. In some embodiments the at least one additional agent is an anti-CD20 antibody (such as rituximab) and one or more chemotherapeutic agents (e.g., gemcitabine and oxaliplatin). In some embodiments, the article of manufacture or kit further comprises package insert comprising instructions for using the anti-CD79b immunoconjugate in conjunction at least one additional agent, such as an anti-CD20 antibody (e.g., rituximab) and one or more chemotherapeutic agents (e.g., gemcitabine and oxaliplatin) to treat or delay progression of a B-cell proliferative disorder (e.g., DLBCL, such as relapsed/refractory DLBCL) in an individual. Any of the anti-CD79b immunoconjugates and anti-cancer agents known in the art may be included in the article of manufacture or kits. In some embodiments, the kit comprises an immunoconjugate comprising the formula

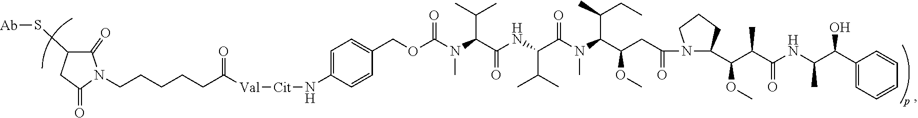

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with an anti-CD20 antibody (such as rituximab) and one or more chemotherapeutic agents (e.g., gemcitabine and oxaliplatin) for treating a human in need thereof having diffuse large B-cell lymphoma (DLBCL). In some embodiments, the kit is for use according to any of the methods provided herein.

In some embodiments, the kit comprises an immunoconjugate comprising the formula

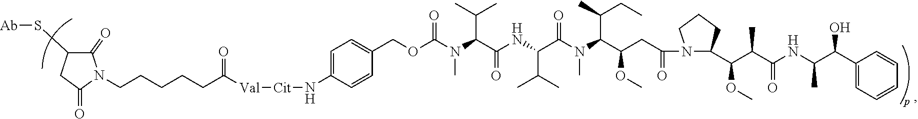

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with rituximab, gemcitabine and oxaliplatin for treating a human in need thereof having diffuse large B-cell lymphoma (DLBCL). In some embodiments, the kit is for use according to any of the methods provided herein.

In some embodiments, the kit comprises an immunoconjugate comprising the formula

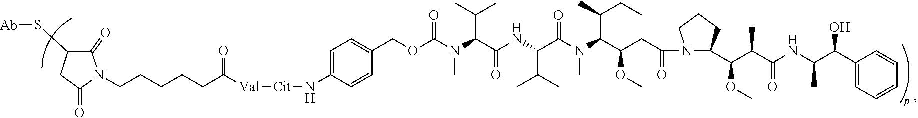

wherein Ab is an anti-CD79b antibody that comprises (i) a heavy chain comprising a VH that comprises the amino acid sequence of SEQ ID NO: 19 and (ii) a light chain comprising a VL that comprises the amino acid sequence of SEQ ID NO: 20, and wherein p is between 2 and 5. In some embodiments, p is between 3 and 4, e.g., 3.5. In some embodiments, the immunoconjugate comprises anti-CD79b antibody comprising a heavy chain comprising the amino acid sequence of SEQ ID NO: 36, and a light chain comprising the amino acid sequence of SEQ ID NO: 35. In certain embodiments, the anti-CD79b immunoconjugate comprises the structure of Ab-MC-vc-PAB-MMAE. In some embodiments, the anti-CD79b immunoconjugate is polatuzumab vedotin-piiq. In some embodiments, the at least one additional agent is an anti-CD20 antibody (such as rituximab) and one or more chemotherapeutic agents (e.g., gemcitabine and oxaliplatin). In some embodiments, the kit is for use in the treatment of DLBCL in an individual (e.g., an individual having one or more characteristics described herein) according to a method provided herein.

In some embodiments, the kit comprises an immunoconjugate comprising the formula

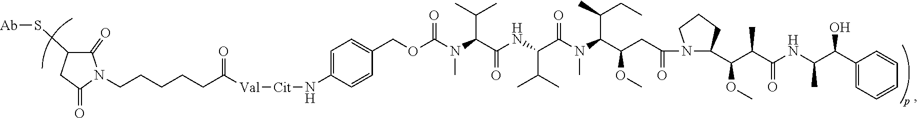

wherein Ab is an anti-CD79b antibody that comprises (i) a heavy chain comprising a VH that comprises the amino acid sequence of SEQ ID NO: 19 and (ii) a light chain comprising a VL that comprises the amino acid sequence of SEQ ID NO: 20, and wherein p is between 2 and 5. In some embodiments, p is between 3 and 4, e.g., 3.5. In some embodiments, the immunoconjugate comprises anti-CD79b antibody comprising a heavy chain comprising the amino acid sequence of SEQ ID NO: 36, and a light chain comprising the amino acid sequence of SEQ ID NO: 35. In certain embodiments, the anti-CD79b immunoconjugate comprises the structure of Ab-MC-vc-PAB-MMAE. In some embodiments, the anti-CD79b immunoconjugate is polatuzumab vedotin-piiq. In some embodiments, the at least one additional agent is rituximab, gemcitabine and oxaliplatin. In some embodiments, the kit is for use in the treatment of DLBCL in an individual (e.g., an individual having one or more characteristics described herein) according to a method provided herein.

In some embodiments, the anti-CD79b immunoconjugate, the anti-CD20 antibody (such as rituximab), and the one or more chemotherapeutic agents (e.g., gemcitabine and oxaliplatin) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

TABLE E
Amino Acid Sequences.

| | | SEQ ID |
|---|---|---|
| NAME | SEQUENCE | NO |
| Human CD79b | RFIARKRGFT VKMHCYMNSA SGNVSWLWKQ | 1 |
| precursor; Acc. | EMDENPQQLK LEKGRMEESQ NESLATLTIQ | |
| No. NP_000617.1; | GIRFEDNGIY FCQQKCNNTS EVYQGCGTEL | |
| signal sequence = | RVMGFSTLAQ LKQRNTLKDG IIMIQTLLII | |
| amino acids 1 | LFIIVPIFLL LDKDDSKAGM EEDHTYEGLD | |
| to 28 | IDQTATYEDI VTLRTGEVKW SVGEHPGQE | |
| Human mature | AR SEDRYRNPKG SACSRIWQSP RFIARKRGFT | 2 |
| CD79b, without | VKMHCYMNSA SGNVSWLWKQ EMDENPQQLK | |
| signal sequence; | LEKGRMEESQ NESLATLTIQ GIRFEDNGIY | |
| amino acids 29 | FCQQKCNNTS EVYQGCGTEL RVMGFSTLAQ | |
| to 229 | LKQRNTLKDG IIMIQTLLII LFIIVPIFLL | |
| | LDKDDSKAGM EEDHTYEGLD IDQTATYEDI | |
| | VTLRTGEVKW SVGEHPGQE | |
| VH of mMAb anti- | Gly Pro Glu Leu Val Lys Pro Gly Ala Ser | |
| CD20 antibody B- | Val Lys Ile Ser Cys Lys Ala Ser Gly Tyr | |
| Ly1 | Ala Phe Ser Tyr Ser Trp Met Asn Trp Val | |
| | Lys Leu Arg Pro Gly Gln Gly Leu Glu Trp | |
| | Ile Gly Arg Ile Phe Pro Gly Asp Gly Asp | |
| | Thr Asp Tyr Asn Gly Lys Phe Lys Gly Lys | |
| | Ala Thr Leu Thr Ala Asp Lys Ser Ser Asn | 3 |
| | Thr Ala Tyr Met Gln Leu Thr Ser Leu Thr | |
| | Ser Val Asp Ser Ala Val Tyr Leu Cys Ala | |
| | Arg Asn Val Phe Asp Gly Tyr Trp Leu Val | |
| | Tyr Trp Gly Gln Gly Thr Leu Val Thr Val | |
| | Ser Ala | |
| VL of mMAb anti- | Asn Pro Val Thr Leu Gly Thr Ser Ala Ser | 4 |
| CD20 antibody B- | Ile Ser Cys Arg Ser Ser Lys Ser Leu Leu | |
| Lyl | His Ser Asn Gly Ile Thr Tyr Leu Tyr Trp | |
| | Tyr Leu Gln Lys Pro Gly Gln Ser Pro Gln | |
| | Leu Leu Ile Tyr Gln Met Ser Asn Leu Val | |
| | Ser Gly Val Pro Asp Arg Phe Ser Ser Ser | |
| | Gly Ser Gly Thr Asp Phe Thr Leu Arg Ile | |
| | Ser Arg Val Glu Ala Glu Asp Val Gly Val | |
| | Tyr Tyr Cys Ala Gln Asn Leu Glu Leu Pro | |
| | Tyr Thr Phe Gly Gly Gly Thr Lys Leu Glu | |
| | Ile Lys Arg | |
| GA101 HVR-H1 | Gly Tyr Ala Phe Ser Tyr | 5 |
| GA101 HVR-H2 | Phe Pro Gly Asp Gly Asp Thr Asp | 6 |
| GA101 HVR-H3 | Asn Val Phe Asp Gly Tyr Trp Leu Val Tyr | 7 |
| GA101 HVR-L1 | Arg Ser Ser Lys Ser Leu Leu His Ser Asn | 8 |
| | Gly Ile Thr Tyr Leu Tyr | |
| GA101 HVR-L2 | Gln Met Ser Asn Leu Val Ser | 9 |
| GA101 HVR-L3 | Ala Gln Asn Leu Glu Leu Pro Tyr Thr | 10 |
| GA101 VH | Gln Val Gln Leu Val Gln Ser Gly Ala Glu | 11 |
| | Val Lys Lys Pro Gly Ser Ser Val Lys Val | |
| | Ser Cys Lys Ala Ser Gly Tyr Ala Phe Ser | |
| | Tyr Ser Trp Ile Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| GA101 VL | Asp Ile Val Met Thr Gln Thr Pro Leu Ser | 12 |
| | Leu Pro Val Thr Pro Gly Glu Pro Ala Ser | |
| | Ile Ser Cys Arg Ser Ser Lys Ser Leu Leu | |
| | His Ser Asn Gly Ile Thr Tyr Leu Tyr Trp | |
| | Tyr Leu Gln Lys Pro Gly Gln Ser Pro Gln | |
| | Leu Leu Ile Tyr Gln Met Ser Asn Leu Val | |
| | Ser Gly Val Pro Asp Arg Phe Ser Gly Ser | |
| | Gly Ser Gly Thr Asp Phe Thr Leu Lys Ile | |
| | Ser Arg Val Glu Ala Glu Asp Val Gly Val | |
| | Tyr Tyr Cys Ala Gln Asn Leu Glu Leu Pro | |
| | Tyr Thr Phe Gly Gly Gly Thr Lys Val Glu | |
| | Ile Lys Arg Thr Val | |
| GA101 Heavy | Gln Val Gln Leu Val Gln Ser Gly Ala Glu | 13 |
| Chain | Val Lys Lys Pro Gly Ser Ser Val Lys Val | |
| | Ser Cys Lys Ala Ser Gly Tyr Ala Phe Ser | |
| | Tyr Ser Trp Ile Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser Ala | |
| | Ser Thr Lys Gly Pro Ser Val Phe Pro Leu | |
| | Ala Pro Ser Ser Lys Ser Thr Ser Gly Gly | |
| | Thr Ala Ala Leu Gly Cys Leu Val Lys Asp | |
| | Tyr Phe Pro Glu Pro Val Thr Val Ser Trp | |
| | Asn Ser Gly Ala Leu Thr Ser Gly Val His | |
| | Thr Phe Pro Ala Val Leu | |
| | Gln Ser Ser Gly Leu Tyr Ser Leu Ser Ser | |
| | Val Val Thr Val Pro Ser Ser Ser Leu Gly | |
| | Thr Gln Thr Tyr Ile Cys Asn Val Asn His | |
| | Lys Pro Ser Asn Thr Lys Val Asp Lys Lys | |
| | Val Glu Pro Lys Ser Cys Asp Lys Thr His | |
| | Thr Cys Pro Pro Cys Pro Ala Pro Glu Leu | |
| | Leu Gly Gly Pro Ser Val Phe Leu Phe Pro | |
| | Pro Lys Pro Lys Asp Thr Leu Met Ile Ser | |
| | Arg Thr Pro Glu Val Thr Cys Val Val Val | |
| | Asp Val Ser His Glu Asp Pro Glu Val Lys | |
| | Phe Asn Trp Tyr Val Asp Gly Val Glu Val | |
| | His Asn Ala Lys Thr Lys Pro Arg Glu Glu | |
| | Gln Tyr Asn Ser Thr Tyr Arg Val Val Ser | |
| | Val Leu Thr Val Leu His Gln Asp Trp Leu | |
| | Asn Gly Lys Glu Tyr Lys Cys Lys Val Ser | |
| | Asn Lys Ala Leu Pro Ala Pro Ile Glu Lys | |
| | Thr Ile Ser Lys Ala Lys Gly Gln Pro Arg | |
| | Glu Pro Gln Val Tyr Thr Leu Pro Pro Ser | |
| | Arg Asp Glu Leu Thr Lys Asn Gln Val Ser | |
| | Leu Thr Cys Leu Val Lys Gly Phe Tyr Pro | |
| | Ser Asp Ile Ala Val Glu Trp Glu Ser Asn | |
| | Gly Gln Pro Glu Asn Asn Tyr Lys Thr Thr | |
| | Pro Pro Val Leu Asp Ser Asp Gly Ser Phe | |
| | Phe Leu Tyr Ser Lys Leu Thr Val Asp Lys | |
| | Ser Arg Trp Gln Gln Gly Asn Val Phe Ser | |
| | Cys Ser Val Met His Glu Ala Leu His Asn | |
| | His Tyr Thr Gln Lys Ser Leu Ser Leu Ser | |
| | Pro Gly | |
| GA101 Light | Asp Ile Val Met Thr Gln Thr Pro Leu Ser | 14 |
| Chain | Leu Pro Val Thr Pro Gly Glu Pro Ala Ser | |
| | Ile Ser Cys Arg Ser Ser Lys Ser Leu Leu | |
| | His Ser Asn Gly Ile Thr Tyr Leu Tyr Trp | |
| | Tyr Leu Gln Lys Pro Gly Gln Ser Pro Gln | |
| | Leu Leu Ile Tyr Gln Met Ser Asn Leu Val | |
| | Ser Gly Val Pro Asp Arg Phe Ser Gly Ser | |
| | Gly Ser Gly Thr Asp Phe Thr Leu Lys Ile | |
| | Ser Arg Val Glu Ala Glu Asp Val Gly Val | |
| | Tyr Tyr Cys Ala Gln Asn Leu Glu Leu Pro | |
| | Tyr Thr Phe Gly Gly Gly Thr Lys Val Glu | |
| | Ile Lys Arg Thr Val Ala Ala Pro Ser Val | |
| | Phe Ile Phe Pro Pro Ser Asp Glu Gln Leu | |
| | Lys Ser Gly Thr Ala Ser Val Val Cys Leu | |
| | Leu Asn Asn Phe Tyr Pro Arg Glu Ala Lys | |
| | Val Gln Trp Lys Val Asp Asn Ala Leu Gln | |
| | Ser Gly Asn Ser Gln Glu Ser Val Thr Glu | |
| | Gln Asp Ser Lys Asp Ser Thr Tyr Ser Leu | |
| | Ser Ser Thr Leu Thr Leu Ser Lys Ala Asp | |
| | Tyr Glu Lys His Lys Val Tyr Ala Cys Glu | |
| | Val Thr His Gln Gly Leu Ser Ser Pro Val | |
| | Thr Lys Ser Phe Asn Arg Gly Glu Cys | |
| VH of humanized | Gln Val Gln Leu Val Gln Ser Gly Ala Glu | 15 |
| B-Ly1 antibody | Val Lys Lys Pro Gly Ser Ser Val Lys Val | |
| (B-HH2) | Ser Cys Lys Ala Ser Gly Tyr Ala Phe Ser | |
| | Tyr Ser Trp Met Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 16 |
| B-Ly1 antibody | Val Lys Lys Pro Gly Ser Ser Val Lys Val | |
| (B-HH3) | Ser Cys Lys Ala Ser Gly Tyr Ala Phe Ser | |
| | Tyr Ser Trp Met Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Leu Cys Ala Arg Asn Val | |
| | Phe Asp Gly Tyr Trp Leu Val Tyr Trp Gly | |
| | Gln Gly Thr Leu Val Thr Val Ser Ser | |
| humanized B-Ly1 | QVQLVQSGAE VKKPGSSVKV SCKASGYAFS | 17 |
| Heavy Chain | YSWINWVRQA PGQGLEWMGR IFPGDGDTDY | |
| | NGKFKGRVTI TADKSTSTAY MELSSLRSED | |
| | TAVYYCARNV FDGYWLVYWG QGTLVTVSSA | |
| | STKGPSVFPL APSSKSTSGG TAALGCLVKD | |
| | YFPEPVTVSW NSGALTSGVH TFPAVLQSSG | |
| | LYSLSSVVTV PSSSLGTQTY ICNVNHKPSN | |
| | TKVDKKVEPK SCDKTHTCPP CPAPELLGGP | |
| | SVFLFPPKPK DTLMISRTPE VTCVVVDVSH | |
| | EDPEVKFNWY VDGVEVHNAK TKPREEQYNS | |
| | TYRVVSVLTV LHQDWLNGKE YKCKVSNKAL | |
| | PAPIEKTISK AKGQPREPQV YTLPPSRDEL | |
| | TKNQVSLTCL VKGFYPSDIA VEWESNGQPE | |
| | NNYKTTPPVL DSDGSFFLYS KLTVDKSRWQ | |
| | QGNVFSCSVM HEALHNHYTQ KSLSLSPG | |
| humanized B-Ly1 | DIVMTQTPLS LPVTPGEPAS ISCRSSKSLL | 18 |
| Light Chain | HSNGITYLYW YLQKPGQSPQ LLIYQMSNLV | |
| | SGVPDRFSGS GSGTDFTLKI SRVEAEDVGV | |
| | YYCAQNLELP YTFGGGTKVE IKRTVAAPSV | |
| | FIFPPSDEQL KSGTASVVCL LNNFYPREAK | |
| | VQWKVDNALQ SGNSQESVTE QDSKDSTYSL | |
| | SSTLTLSKAD YEKHKVYACE VTHQGLSSPV | |
| | TKSFNRGEC | |
| huMA79bv28 heavy | EVQLVESGGG LVQPGGSLRL SCAASGYTFS | 19 |
| chain variable | SYWIEWVRQA PGKGLEWIGE ILPGGGDTNY | |
| region | NEIFKGRATF SADTSKNTAY LQMNSLRAED | |
| | TAVYYCTRRV PIRLDYWGQG TLVTVSS | |
| huMA79bv28 light | DIQLTQSPSS LSASVGDRVT ITCKASQSVD | 20 |
| chain variable | YEGDSFLNWY QQKPGKAPKL LIYAASNLES | |
| region | GVPSRFSGSG SGTDFTLTIS SLQPEDFATY | |
| | YCQQSNEDPL TFGQGTKVEI KR | |
| huMA79bv28 HVR | GYTFSSYWIE | 21 |
| H1 | | |
| huMA79bv28 HVR | GEILPGGGDTNYNEIFKG | 22 |
| H2 | | |
| huMA79bv28 HVR | TRRVPIRLDY | 23 |
| H3 | | |
| huMA79bv28 HVR | KASQSVDYEGDSFLN | 24 |
| L1 | | |
| huMA79bv28 HVR | AASNLES | 25 |
| L2 | | |
| huMA79bv28 HVR | QQSNEDPLT | 26 |
| L3 | | |
| huMA79bv28 heavy | EVQLVESGGGLVQPGGSLRLSCAAS | 27 |
| chain (HC) | | |
| framework region | | |
| (FR) 1 | | |
| huMA79bv28 HC | WVRQAPGKGLEWI | 28 |
| FR2 | | |
| huMA79bv28 HC | RATFSADTSKNTAYLQMNSLRAEDTAVYYC | 29 |
| FR3 | | |
| huMA79bv28 HC | WGQGTLVTVSS | 30 |
| FR4 | | |
| huMA79bv28 light | DIQLTQSPSSLSASVGDRVTITC | 31 |
| chain (LC) FR1 | | |
| huMA79bv28 LC | WYQQKPGKAPKLLIY | 32 |
| FR2 | | |
| huMA79bv28 LC | GVPSRFSGSGSGTDFTLTISSLQPEDFATYYC | 33 |
| FR3 | | |
| huMA79bv28 LC | FGQGTKVEIKR | 34 |
| FR4 | | |
| huMA79bv28 light | DIQLTQSPSS LSASVGDRVT ITCKASQSVD | 35 |
| chain (Igκ) | YEGDSFLNWY QQKPGKAPKL LIYAASNLES | |
| | GVPSRFSGSG SGTDFTLTIS SLQPEDFATY | |
| | YCQQSNEDPL TFGQGTKVEI KRTVAAPSVF | |
| | IFPPSDEQLK SGTASVVCLL NNFYPREAKV | |
| | QWKVDNALQS GNSQESVTEQ DSKDSTYSLS | |
| | STLTLSKADY EKHKVYACEV THQGLSSPVT | |
| | KSFNRGEC | |
| huMA79bv28 heavy | EVQLVESGGG LVQPGGSLRL SCAASGYTFS | 36 |
| chain (IgG1) | SYWIEWVRQA PGKGLEWIGE ILPGGGDTNY | |
| | NEIFKGRATF SADTSKNTAY LQMNSLRAED | |
| | TAVYYCTRRV PIRLDYWGQG TLVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKKVEPKSC DKTHTCPPCP APELLGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| huMA79bv28 A118C | EVQLVESGGG LVQPGGSLRL SCAASGYTFS | 37 |
| cysteine | SYWIEWVRQA PGKGLEWIGE ILPGGGDTNY | |
| engineered heavy | NEIFKGRATF SADTSKNTAY LQMNSLRAED | |
| chain (IgG1) | TAVYYCTRRV PIRLDYWGQG TLVTVSSCST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKKVEPKSC DKTHTCPPCP APELLGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| huMA79bv28 V205C | DIQLTQSPSS LSASVGDRVT ITCKASQSVD | 38 |
| cysteine | YEGDSFLNWY QQKPGKAPKL LIYAASNLES | |
| engineered light | GVPSRFSGSG SGTDFTLTIS SLQPEDFATY | |
| chain (Igκ) | YCQQSNEDPL TFGQGTKVEI KRTVAAPSVF | |
| | IFPPSDEQLK SGTASVVCLL NNFYPREAKV | |
| | QWKVDNALQS GNSQESVTEQ DSKDSTYSLS | |
| | STLTLSKADY EKHKVYACEV THQGLSSPCT | |
| | KSFNRGEC | |
| huMA79bv28 S400C | EVQLVESGGG LVQPGGSLRL SCAASGYTFS | 39 |
| cysteine | SYWIEWVRQA PGKGLEWIGE ILPGGGDTNY | |
| engineered heavy | NEIFKGRATF SADTSKNTAY LQMNSLRAED | |
| chain (IgG1) | TAVYYCTRRV PIRLDYWGQG TLVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKKVEPKSC DKTHTCPPCP APELLGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDC DGSFFLYSKL TVDKSRWQQG | |
| | NVFSCSVMHE ALHNHYTQKS LSLSPGK | |
| VH of humanized | Gln Val Gln Leu Val Gln Ser Gly Ala Glu | 40 |
| B-Ly1 antibody | Val Lys Lys Pro Gly Ala Ser Val Lys Val | |
| (B-HH4) | Ser Cys Lys Val Ser Gly Tyr Ala Phe Ser | |
| | Tyr Ser Trp Met Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 41 |
| B-Ly1 antibody | Val Lys Lys Pro Gly Ser Ser Val Lys Val | |
| (B-HH5) | Ser Cys Lys Ala Ser Gly Tyr Ala Phe Ser | |
| | Tyr Ser Trp Met Ser Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 42 |
| B-Ly1 antibody | Val Lys Lys Pro Gly Ser Ser Val Lys Val | |
| (B-HH6) | Ser Cys Lys Ala Ser Gly Tyr Ala Phe Ser | |
| | Tyr Ser Trp Ile Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 43 |
| B-Ly1 antibody | Val Lys Lys Pro Gly Ser Ser Val Lys Val | |
| (B-HH7) | Ser Cys Lys Ala Ser Gly Tyr Ala Phe Ser | |
| | Tyr Ser Trp Ile Ser Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 44 |
| B-Ly1 antibody | Val Lys Lys Pro Gly Ala Ser Val Lys Val | |
| (B-HH8) | Ser Cys Lys Ala Ser Gly Tyr Thr Phe Thr | |
| | Tyr Ser Trp Met Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Gln Ser Gly Ala Glu | 45 |
| B-Ly1 antibody | Val Lys Lys Pro Gly Ala Ser Val Lys Val | |
| (B-HH9) | Ser Cys Lys Ala Ser Gly Tyr Thr Phe Ser | |
| | Tyr Ser Trp Met Asn Trp Val Arg Gln Ala | |
| | Pro Gly Gln Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 46 |
| B-Ly1 antibody | Leu Val Lys Pro Gly Gly Ser Leu Arg Leu | |
| (B-HL8) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Val Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 47 |
| B-Ly1 antibody | Leu Val Lys Pro Gly Gly Ser Leu Arg Leu | |
| (B-HL10) | Ser Cys Ala Ala Ser Gly Phe Ala Phe Ser | |
| | Tyr Ser Trp Met Asn Trp Val Arg Gln Ala | |
| | Pro Gly Lys Gly Leu Glu Trp Val Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Gln Val Gln Leu Val Glu Ser Gly Gly Gly | 48 |
| B-Ly1 antibody | Leu Val Lys Pro Gly Gly Ser Leu Arg Leu | |
| (B-HL11) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Val Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Ala Gly | 49 |
| B-Ly1 antibody | Leu Val Lys Pro Gly Gly Ser Leu Arg Leu | |
| (B-HL12) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 50 |
| B-Ly1 antibody | Val Val Lys Pro Gly Gly Ser Leu Arg Leu | |
| (B-HL13) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 51 |
| B-Ly1 antibody | Leu Lys Lys Pro Gly Gly Ser Leu Arg Leu | |
| (B-HL14) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 52 |
| B-Ly1 antibody | Leu Val Lys Pro Gly Ser Ser Leu Arg Leu | |
| (B-HL15) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 53 |
| B-Ly1 antibody | Leu Val Lys Pro Gly Gly Ser Leu Arg Val | |
| (B-HL16) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly Thr Leu Val Thr Val Ser Ser | |
| VH of humanized | Glu Val Gln Leu Val Glu Ser Gly Gly Gly | 54 |
| B-Ly1 antibody | Leu Val Lys Pro Gly Gly Ser Leu Arg Leu | |
| (B-HL17) | Ser Cys Ala Ala Ser Gly Phe Thr Phe Ser | |
| | Tyr Ser Trp Met Asn Trp Val Arg Gln Ala | |
| | Pro Gly Lys Gly Leu Glu Trp Met Gly Arg | |
| | Ile Phe Pro Gly Asp Gly Asp Thr Asp Tyr | |
| | Asn Gly Lys Phe Lys Gly Arg Val Thr Ile | |
| | Thr Ala Asp Lys Ser Thr Ser Thr Ala Tyr | |
| | Met Glu Leu Ser Ser Leu Arg Ser Glu Asp | |
| | Thr Ala Val Tyr Tyr Cys Ala Arg Asn Val | |
| | Phe Asp Gly Tyr Trp Leu Val Tyr Trp Gly | |
| | Gln Gly ThrLeu Val Thr Val Ser Ser | |
| VL of humanized | Asp Ile Val Met Thr Gln Thr Pro Leu Ser | 55 |
| B-Ly1 antibody | Leu Pro Val Thr Pro Gly Glu Pro Ala Ser | |
| (B-KVI) | Ile Ser Cys Arg Ser Ser Lys Ser Leu Leu | |
| | His Ser Asn Gly Ile Thr Tyr Leu Tyr Trp | |
| | Tyr Leu Gln Lys Pro Gly Gln Ser Pro Gln | |
| | Leu Leu Ile Tyr Gln Met Ser Asn Leu Val | |
| | Ser Gly Val Pro Asp Arg Phe Ser Gly Ser | |
| | Gly Ser Gly Thr Asp Phe Thr Leu Lys Ile | |
| | Ser Arg Val Glu Ala Glu Asp Val Gly Val | |
| | Tyr Tyr Cys Ala Gln Asn Leu Glu Leu Pro | |
| | Tyr Thr Phe Gly Gly Gly Thr Lys Val Glu | |
| | Ile Lys Arg Thr Val | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following are examples of methods and compositions of the disclosure. It is understood that various other embodiments may be practiced, given the general description provided above.

### Example 1: A Phase III, Open-Label, Multicenter, Randomized Study Evaluating the Safety and Efficacy of Polatuzumab Vedotin in Combination with Rituximab Plus Gemcitabine Plus Oxaliplatin (Pola-R-GemOx) Versus R-GemOx Alone in Patients with Relapsed/Refractory Diffuse Large B Cell Lymphoma

This example describes a Phase III, open-label, multicenter, randomized study evaluating the safety and efficacy of polatuzumab vedotin in combination with rituximab, gemcitabine and oxaliplatin (Pola-R-GemOx) compared to rituximab, gemcitabine and oxaliplatin alone (R-GemOx) in patients with Relapsed/Refractory (R/R) Diffuse Large B Cell Lymphoma (DLBCL). In this study, treatment occurs in two stages: 1) an initial safety run-in stage assessing the safety of the Pola-R-GemOx combination (Stage 1); and 2) a randomized controlled trial (RCT) stage comparing the safety and efficacy of Pola-R-GemOx versus R-GemOx (Stage 2).

### I. Study Objectives

### A. Stage 1: Safety Run-In

Safety Objectives and Endpoints

Primary Safety Objective

The primary safety objective of Stage 1 of this study is to evaluate the safety and tolerability of Pola-R-GemOx as a combination therapy.

Primary Safety Endpoints

The primary safety endpoints of Stage 1 of this study are the incidence, nature and severity of physical findings and adverse events (AEs), with a specific focus on peripheral neuropathy, according to the National Cancer Institute Common Terminology Criteria for Adverse Events, Version 5 (NCI CTCAE v5.0).

Secondary Safety Objectives

The secondary safety objectives of Stage 1 of this study are to evaluate the safety and tolerability of Pola-R-GemOx as a combination therapy and to assess the immunogenicity of polatuzumab vedotin.

Secondary Safety Endpoints

The secondary safety endpoints of Stage 1 of this study are the incidence and assessment of peripheral neuropathy, as measured by the Functional Assessment of Cancer Therapy/Gynecologic Oncology Group-Neurotoxicity 12-Item Scale (FACT-GOG/Ntx12); the tolerability of Pola-R-GemOx, as measured by dose interruptions, dose reductions and dose intensity; and the prevalence of anti-drug antibodies (ADAs) at baseline and incidence of ADAs during the study.

Efficacy Objectives and Endpoints

Secondary Efficacy Objective

The secondary efficacy objective of Stage 1 of this study is to evaluate the efficacy of Pola-R-GemOx.

Secondary Efficacy Endpoint

The secondary efficacy endpoints of Stage 1 of this study are as follows:
Complete response (CR), defined as the proportion of patients who achieve complete metabolic response based on PET-CT, according to Lugano 2014 response criteria (Cheson et al., (2014) J Clin Oncol 32:3059-3068), at the end of treatment.
Objective response rate (ORR), defined as the proportion of patients who achieve complete or partial metabolic responses (based on response including PET CT data), according to Lugano 2014 response criteria, at end of treatment.
Best overall response (BOR), defined as the best response recorded from the start of treatment until end of treatment, according to Lugano 2014 response criteria.
Progression-free survival (PFS), defined as the time from the first dose of study treatment to the first occurrence of disease progression (based on response including PET CT data or not including any PET data) according to Lugano 2014 response criteria, or death from any cause.
Overall Survival (OS), defined as the time from first dose of study treatment to death from any cause.
Event-free survival (EFS_{eff}), defined as time from the first dose of study treatment to the earliest occurrence of any of the following:
   Disease progression or relapse (based on response including PET CT data or not including any PET data).
   Death due to any cause.
   Initiation of any non-protocol-specified anti-lymphoma treatment (NALT).

Pharmacokinetic Objective and Endpoints

Pharmacokinetic Objective

The pharmacokinetic objective of Stage 1 of this study is to evaluate the pharmacokinetic (PK) profile of polatuzumab vedotin.

Pharmacokinetic Endpoints

The pharmacokinetic endpoints of Stage 1 of this study are the PK profile of polatuzumab vedotin in combination with R-GemOx in patients with relapsed or refractory DLBCL and potential PK interactions between polatuzumab vedotin and R-GemOx.

### B. Stage 2: Randomized Controlled Trial (RCT)

Efficacy Objectives and Endpoints

Primary and Secondary Efficacy Objective

The primary and secondary efficacy objective of Stage 2 of this study is to evaluate the efficacy of Pola-R-GemOx compared with R-GemOx alone.

Primary Efficacy Endpoint

The primary efficacy endpoint of Stage 2 of this study is overall survival (OS), defined as time from randomization to death from any cause.

Secondary Efficacy Endpoints

The secondary efficacy endpoints of Stage 2 of this study are as follows:
Key secondary endpoints included in the hierarchical testing procedure (See Section V, below):
   PFS, defined as the time from randomization to the first occurrence of disease progression, according to Lugano 2014 response criteria, or death from any cause.
   CRR, defined as the proportion of patients who achieve complete metabolic response based on PET-CT, according to Lugano 2014 response criteria, at the end of treatment.
   ORR, defined as the proportion of patients who achieve complete or partial metabolic responses, according to Lugano 2014 response criteria, at the end of treatment.
Secondary endpoints that are not adjusted for testing multiplicity procedure:
   BOR, defined as the best response recorded from randomization until end of treatment, according to Lugano 2014 response criteria.
   CRR, defined as the proportion of patients who achieve complete metabolic response based on PET-CT, according to Lugano 2014 response criteria, at the end of treatment.
   ORR, defined as the proportion of patients who achieve complete or partial metabolic responses (based on response including PET CT data), according to Lugano 2014 response criteria, at the end of treatment.
   Duration of objective response (DoR), defined as the time from the first occurrence of a documented objective response (based on response including PET CT data) to disease progression (based on response including PET CT data or not including any PET data), according to Lugano 2014 response criteria, or death from any cause, whichever occurs first.
   EFSeff, defined as time from randomization to the earliest occurrence of any of the following:
      Disease progression or relapse (based on response including PET CT data or not including any PET data).
      Death due to any cause.
      Initiation of any NALT.

Patient Reported Outcome (PRO) Objectives and Endpoints

Secondary and Exploratory PRO Objectives

The secondary PRO objectives of Stage 2 of this study are to evaluate the impact of treatment and disease on aspects of health-related quality of life.

Secondary PRO Endpoints

The secondary PRO endpoints of Stage 2 of this study are as follows:
Time to deterioration in physical functioning and fatigue as measured by the European Organisation for the Research and Treatment of Cancer Quality-of-Life Questionnaire, Core 30 (EORTC QLQ-C30).
Time to progression in lymphoma symptoms as measured by the Functional Assessment of Cancer Therapy-Lymphoma (FACT-Lym) subscale.
Change from baseline in peripheral neuropathy as measured by the FACT/GOG-NTX-12 subscale score.

Exploratory PRO Endpoints
The exploratory PRO endpoints of Stage 2 of this study are descriptive summary statistics and the change from baseline for all scales for the EORTC QLQ-C30, FACT-Lym subscale, FACT/GOG-NTX-12, and EQ-5D-5L.

Safety Objectives and Endpoints

Safety Objectives

The safety objectives of Stage 2 of this study are to evaluate the safety and tolerability of Pola-R-GemOx compared with R-GemOx and to assess the immunogenicity of polatuzumab vedotin.

Safety Endpoints

The safety endpoints of Stage 2 of this study are as follows:
Incidence, nature and severity of AEs (including peripheral neuropathy) according to NCI CTCAE v5.0 and physical findings.
Tolerability, as assessed by dose interruptions, dose reductions and dose intensity.
Incidence and assessment of peripheral neuropathy, as measured by the FACT-GOG/Ntx12.
Prevalence of anti-drug antibodies (ADAs) at baseline and incidence of ADAs during the study.

Biomarker Objectives and Endpoints Exploratory Biomarker Objectives

The exploratory biomarker endpoints of Stage 2 of this study are to identify biomarkers that meet the following criteria:
Are prognostic of response to polatuzumab vedotin (i.e., predictive biomarkers).
Are associated with progression to a more severe disease (i.e., prognostic biomarkers).
Provide evidence of polatuzumab vedotin activity, or increase the knowledge and understanding of disease biology.

Exploratory Biomarker Endpoints

The exploratory biomarker objectives of Stage 2 of this study are the associations between efficacy endpoints including overall survival (OS), progression-free survival (PFS), and complete response (CR) rate, and exploratory biomarkers, such as molecular DLBCL prognostic cell of origin subtypes, BCL2 and MYC double expresser, and key lymphoma mutations.

Pharmacokinetic Objectives and Endpoints

Pharmacokinetic Objective

The pharmacokinetic objective of Stage 2 of this study is to evaluate the pharmacokinetic (PK) profile of polatuzumab vedotin.

Pharmacokinetic Endpoints

The pharmacokinetic endpoints of Stage 2 of this study are as follows:
PK profile of polatuzumab vedotin in combination with R-GemOx in patients with relapsed or refractory DLBCL.
Potential PK interactions between polatuzumab vedotin and R-GemOx.

### II. Patients

Approximately 10 patients are enrolled in the safety run-in phase (Stage 1), and approximately 206 patients are enrolled in the randomized portion (Stage 2), for a total of approximately 216 patients.

### A. Inclusion Criteria

Patients are included in this study if they meet the following criteria:
Age ≥18 years.
Histologically confirmed diffuse large B-cell lymphoma, not otherwise specified (NOS), or history of transformation of indolent disease to DLBCL.
At least one (≥1) line of prior systemic therapy.
   Prior autologous hematopoietic stem cell transplantation (HSCT) is allowed; chemotherapy followed by consolidative autologous HSCT is counted as one line of therapy.
   Prior Allogeneic HSCT is allowed, so long as patients are off all immunosuppressive therapy and have no active graft versus host disease (GVHD); chemotherapy followed by allogeneic HSCT is counted as one line of therapy.
   Local therapies (e.g., radiotherapy) are not considered as lines of treatment.
Relapsed or refractory disease, defined as follows:
   Relapsed: Disease that recurs following a response that lasted ≥6 months from completion of the last line of therapy.
   Refractory: Disease that progresses during therapy or progresses within 6 months (<6 months) of prior therapy.
At least one bi-dimensionally measurable lesion, defined as >1.5 cm in its longest dimension as measured by CT or MRI.
Eastern Cooperative Oncology Group (ECOG) performance status of 0, 1 or 2.
   Patients with an ECOG performance status of 3 are considered for inclusion in in the RCT stage (Stage 2), but only if this status is DLBCL-related, and after a prephase of a 7-day steroid treatment during the screening phase (e.g., 1 mg/kg prednisone). A subsequent improvement in ECOG performance status to 2 or less must be observed to be eligible to be enrolled in the study.
Adequate hematological function, as defined by:
   Hemoglobin ≥8 g/dL.
   Absolute neutrophil count (ANC) ≥1.5×10⁹/L or ≥0.5×10⁹ if the neutropenia is attributable to underlying disease and before administration of steroids.
   Platelet count ≥75×10⁹/L or ≥50×10⁹ if the thrombocytopenia is attributable to underlying disease.
For women of childbearing potential: agreement to remain abstinent or use contraceptive measures, and agreement to refrain from donating eggs.
For men: agreement to remain abstinent or use contraceptive measures, and agreement to refrain from donating sperm.

### B. Exclusion Criteria

Patients are excluded from this study if they meet the following criteria:
History of severe allergic or anaphylactic reactions to humanized or murine monoclonal antibodies (or recombinant antibody-related fusion proteins), or known sensitivity or allergy to murine products.
Contraindication to rituximab, gemcitabine or oxaliplatin.
Peripheral neuropathy assessed to be ≥Grade 1 according to NCI CTCAE v5.0 at enrollment.
Prior use of polatuzumab vedotin or a gemcitabine plus a platinum-based agent combination.
Enrollment in any previous or ongoing polatuzumab vedotin trial.
Treatment with radiotherapy, chemotherapy, immunotherapy, immunosuppressive therapy, or any investigational agent for the purposes of treating cancer within 2 weeks prior to Cycle 1 Day 1.
   All acute, clinically significant treatment-related toxicities from prior therapy, except for alopecia, must have resolved to <Grade 2 prior to Cycle 1 Day 1.
Planned autologous or allogeneic stem cell transplantation at time of recruitment.
   Patients with only one prior therapy who are appropriate for stem cell transplantation are excluded from this trial. Reasons for transplant-ineligibility include age, performance status, comorbidities, transplant failure or failed procedure, insufficient response to salvage therapy, patient refusal, or logistical reasons.
Primary or secondary central nervous system (CNS) lymphoma at the time of recruitment.
Richter's transformation or prior capillary leak syndrome (CLL).
Any of the following abnormal laboratory values, unless abnormal laboratory values are due to underlying lymphoma:
   Creatinine >1.5×upper limit of normal (ULN) or a measured creatinine clearance <30 mL/min.
   Aspartate aminotransferase (AST) or alanine aminotransferase (ALT) >2.5×ULN.
   Total bilirubin ≥1.5×ULN; patients with documented Gilbert disease are enrolled if total bilirubin is ≤3×ULN.
   International normalized ratio (INR) or prothrombin time (PT) >1.5×ULN in the absence of therapeutic anticoagulation.
   Partial thromboplastin time (PTT) or activated partial thromboplastin time (aPTT) >1.5×ULN in the absence of a lupus anticoagulant.
History of other malignancy that could affect compliance with the protocol or interpretation of results. Exceptions include:
   Patients with a history of curatively treated basal or squamous cell carcinoma of the skin or in situ carcinoma of the cervix at any time prior to the study are eligible.
   A patient with any other malignancy appropriately treated with curative intent (and the malignancy has been in remission without treatment for ≥2 years prior to enrollment) is eligible.
   Patients with low-grade, early-stage prostate cancer (Gleason score 6 or below, Stage 1 or 2) with no requirement for therapy at any time prior to study are eligible.
Evidence of significant, uncontrolled concomitant diseases that could affect compliance with the protocol or interpretation of results, including significant cardiovascular disease (such as New York Heart Association [NYHA] Class III or IV cardiac disease, myocardial infarction within the last 6 months, unstable arrhythmias, or unstable angina) or significant pulmonary disease (including obstructive pulmonary disease and history of bronchospasm).
Known active bacterial, viral, fungal, mycobacterial, parasitic, or other infection (excluding fungal infections of nail beds) at study enrollment, or any major episode of infection within 4 weeks prior to Cycle 1 Day 1.
Patients with suspected or latent tuberculosis; latent tuberculosis is confirmed by positive interferon-gamma release assay.
Positive test results for chronic hepatitis B virus (HBV) infection (defined as positive hepatitis B surface antigen [HBsAg] serology).
   Patients with occult or prior HBV infection (defined as negative HBsAg and positive hepatitis B core antibody [HBcAb]) are included if HBV DNA is undetectable, provided that they undergo DNA testing on day 1 of every cycle and monthly for at least 12 months after the last cycle of study treatment.
Positive test results for hepatitis C virus (HCV) antibody.
   Patients who are positive for HCV antibody are eligible only if polymerase chain reaction (PCR) is negative for HCV RNA.
Known history of human immunodeficiency virus (HIV) seropositive status.

For patients with unknown HIV status, HIV testing is performed at Screening if required by local regulations.
Vaccination with a live vaccine within 4 weeks prior to treatment.
Recent major surgery (within 6 weeks before the start of Cycle 1 Day 1) other than for diagnosis.
Any other diseases, metabolic dysfunction, physical examination finding, or clinical laboratory finding giving reasonable suspicion of a disease or condition that contraindicates the use of an investigational drug or that may affect the interpretation of the results or render the patient at high risk from treatment complications.
Pregnant or breastfeeding, or intending to become pregnant during the study or within 12 months after the last dose of study drug.

### III. Study Design

This study is a Phase III, multicenter, open-label, randomized study in patients with relapsed or refractory DLBCL. The study consists of a Screening Period, a Treatment Period (Stage 1 and Stage 2), and a Post-Treatment Period. The Post-Treatment Period includes an End of Treatment Visit occurring 28 days after the last dose of study treatment and a Follow-Up Period. Adverse events, serious adverse events and adverse events of special interest are reported until 90 days after the last dose of study drug or until initiation of a non-protocol-specified anti-lymphoma treatment (NALT).

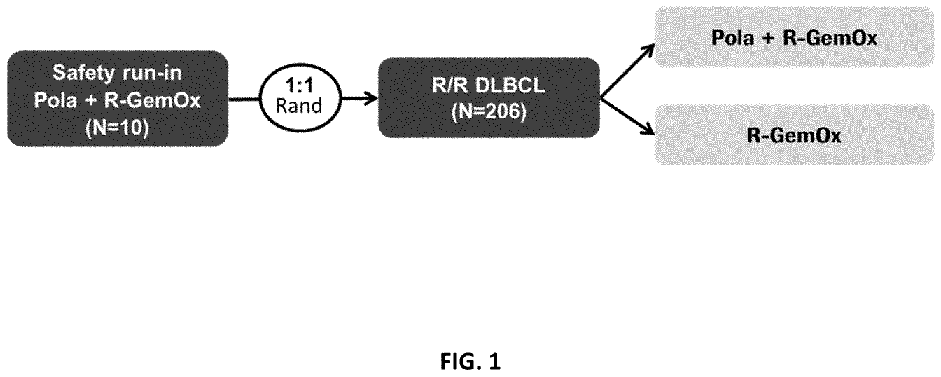

The overall design of the study is presented in FIG. 1.

### A. Screening Period

To be eligible for this study, patients must have histologically confirmed, relapsed or refractory DLBCL.

Patients may have undergone autologous hematopoietic stem cell transplantation (HSCT) prior to recruitment; chemotherapy followed by consolidative autologous HSCT is counted as one line of therapy. Patients with a prior allogeneic HSCT are enrolled so long as they are off all immunosuppressive therapy and have no active graft versus host disease (GVHD); chemotherapy followed by allogeneic HSCT is counted as one line of therapy. Local therapies (e.g., radiotherapy) are not considered as treatment lines. Patients with peripheral neuropathy assessed to be greater than 1 according to NCI CTCAE v5.0 are excluded from enrollment. Other inclusion and exclusion criteria are provided in Section II, above.

### B. Treatment Period

The Treatment Period occurs in two stages, a safety run-in stage (Stage 1) and a randomized controlled trial (Stage 2).

In Stage 1, the safety run-in, 10 patients receive experimental study treatment with Pola-R-GemOx.

In Stage 2, the randomized controlled trial (RCT), patients are randomly assigned in a 1:1 ratio to receive either experimental study treatment with Pola-R-GemOx or control study treatment with R-GemOx.

The primary objective of the randomized part of this study (Stage 2) is to evaluate the efficacy of Pola-R-GemOx versus R-GemOx in patients with relapsed or refractory DLBCL as measured by OS. Assuming a median OS of 9.5 months in the R-GemOx arm (Mounier et al., (2013) Haematologica, 98:1726-1731) and a randomization ratio of 1:1, 121 events are required to detect a between-group difference of 6.3 months in the median OS (hazard ratio [HR]=0.60) with 80% power and a 2-sided a of 0.05. Based on the above statistical assumptions, and anticipating a recruitment period of approximately 11 months (18 patients per month) and a follow-up of 14 months after the last patient is randomized, a total of approximately 206 patients are included in Stage 2 of this study, taking into account an estimated drop-out rate of 10%. The patients are randomized in a ratio of 1:1 into the Pola-R-GemOx and R-GemOx treatments.

In both stages of the Treatment Period, patients receive up to 8 cycles of Pola-R-GemOx or 8 cycles of R-GemOx, each administered on 21-day cycles.

Stage 1: Safety Run-in

A total of 10 patients are treated with Pola-R-GemOx. The 10 patients are staggered across three cohorts:
Cohort 1: First 3 patients.
Cohort 2: Second 3 patients.
Cohort 3: Final 4 patients.

Within each cohort, safety is evaluated at the end of the fourth cycle in all subjects, with a focus on acute peripheral neuropathy toxicities. Once all safety evaluations are conducted within a cohort and further subject accrual is cleared, the next cohort opens to recruitment. This procedure continues until all three cohorts have been fully recruited.

Cohort 2 and Cohort 3 are not enrolled until all safety evaluations have been completed in Cohort 1 and Cohort 2, respectively, and recruitment of the next cohort is approved. If a patient within a cohort discontinues prior to Cycle 4 of treatment due to disease progression, death or any other reason that is not directly attributable to peripheral neuropathy, then the patient is replaced.

Once the entire population of a cohort completes at least four cycles, the total safety profile of Pola-R-GemOx, with a particular focus on peripheral neuropathy, is evaluated and a recommendation of whether the next cohort opens is provided (go/no-go decision). Potential grounds for stopping further enrollment into the next cohort are if >33.33% of patients within a cohort have ≥Grade 3 peripheral neuropathy that does not resolve to ≤Grade 1 within 14 days. The potential beneficial effects of dose modification, the time course of resolution of the peripheral neuropathy events, etc., are considered in the go/no-go decision. In addition, at the safety evaluations for Cohort 2 and Cohort 3, all patients from previous cohorts who proceed beyond the fourth cycle of therapy are re-evaluated to assess the potential of cumulative neurotoxicity with Pola-R-GemOx, as well as the course of peripheral neuropathy resolution.

Stage 1 also assesses ADAs and the sparse PK profile of polatuzumab vedotin in combination with R-GemOx.

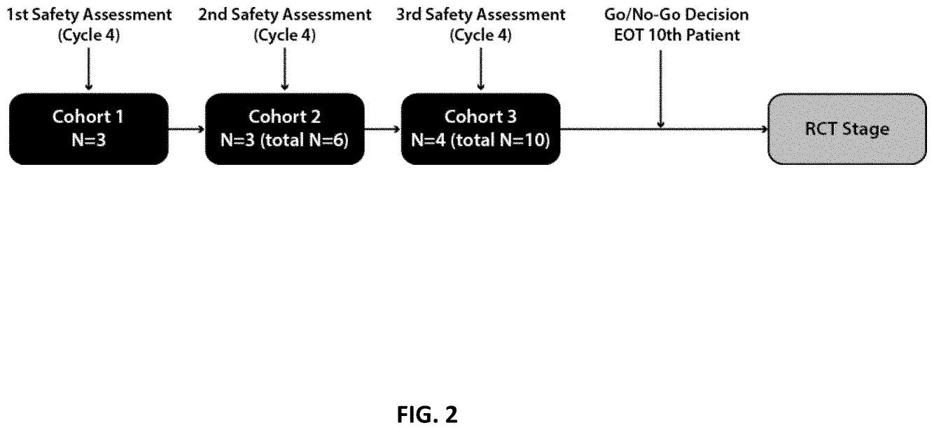

A summary of the Safety Run-In (Stage 1) of this study is provided in FIG. 2.

After the tenth subject completes the entire Treatment period (FIG. 2), the safety and tolerability of the Pola-R-GemOx regimen is assessed, and a decision (Go/No-Go Decision) (FIG. 2) is made whether to continue into Stage 2 (RCT stage) of the study at the end of treatment for the tenth patient. The formal Go/No-Go decision at the end of Stage 1 is determined as follows:
When the last patient remaining in the Treatment Period of Stage 1 has received the last dose of Pola-R-GemOx, the entire safety profile of the Stage 1 study population is reviewed, focusing in particular on the frequency, course and reversibility of Grade ≥3 peripheral neuropathy events:
   If 3 or fewer patients in Stage 1 (n=10) have ≥Grade 3 peripheral neuropathy during the Treatment Period that fails to resolve to ≤Grade 1 within 14 days, the study continues into the Stage 2, the randomized controlled trial (RCT) stage.
   If 4 or more patients in Stage 1 (n=10) have ≥Grade 3 peripheral neuropathy during the Treatment Period that fails to resolve to ≤Grade 1 within 14 days, the study is put on hold.
   Whether the risk of peripheral neuropathy at original study treatment doses is found to be satisfactorily mitigated following dose modification is assessed.

Based on the preceding information, a recommendation of whether to continue into Stage 2, the RCT Stage, is provided.

Stage 2: Randomized Controlled Trial

If Pola-R-GemOx combination therapy is deemed tolerable in the safety run-in (Stage 1), enrollment into the RCT stage begins. A total of 206 eligible patients are randomized in a 1:1 ratio to receive either Pola-R-GemOx (Experimental Treatment) or R-GemOx (Control).

Randomization is performed is by IxRS using stratified permuted blocks. Stratification factors include: 1) Number of previous lines of systemic treatment or therapy (1 vs. ≥2); 2) Outcome of last systemic treatment or therapy (relapsed vs. refractory); and 3) Age (≤70 years vs. >70 years). For stratification based on the number of previous lines of systemic treatment or therapy: chemotherapy followed by consolidative autologous HSCT is counted as one line of therapy; chemotherapy followed by allogeneic HSCT is counted as one line of therapy; local therapies (e.g., radiotherapy) are not considered as lines of treatment. For stratification based on the outcome of the last systemic therapy or treatment: relapsed disease is defined as disease that recurs ≥6 months after completion of the last line of treatment; refractory disease is defined as disease that progresses during therapy or progresses within 6 months (<6 months) of prior therapy.

The stratification produces treatment groups that are roughly equivalent in terms of disease progression and disease status, thereby ensuring comparability between the experimental (Pola-R-GemOx) and control (R-GemOx) arms.

Interim safety analyses are performed during the RCT stage. First and second safety interim analyses are performed after the first 10 and 20 patients, respectively, are randomized in each arm and complete at least 2 cycles of treatment. Thereafter, the frequency of interim safety assessments depends on the number of ≥Grade 3 peripheral neuropathy events observed during the second safety interim assessment. Peripheral neuropathy ≥Grade 3 that recovers to ≤Grade 1 within 14 days is not taken into account for the stopping rules.

Safety is assessed by adverse events per NCI CTCAE v5.0, FACT/GOG-Ntx12 score, clinical laboratory test results, electrocardiogram (ECG) and vital signs. Response assessments are according to Lugano 2014 criteria, based on positron emission tomography-computed tomography (PET-CT) scans at the end of treatment.

Stage 2 assesses ADAs and the sparse PK profile of polatuzumab vedotin in combination with R-GemOx. In addition, Stage 2 assesses biomarkers and patient-reported outcomes.

### C. End of Study and Length of Study

After the initial safety-run in, recruitment for the randomized stage occurs over approximately 11 months.

This is an event-driven trial. The clinical cut-off date for the final OS analysis is confirmed when the targeted number of mortality events (121 deaths) occur, which is expected approximately 25 months after the first patient is randomized (first patient in [FPI]) in the RCT stage of the study.

The length of the study and the time for final analysis depends on the recruitment rate and the number of events that occur. Mortality events are monitored throughout the course of the study, and study timelines are updated.

### IV. Doses and Timing of Administration of Investigational Medicinal Products

The investigational medicinal product (IMP) for this study is Pola-R-GemOx. The control therapy is R-GemOx.

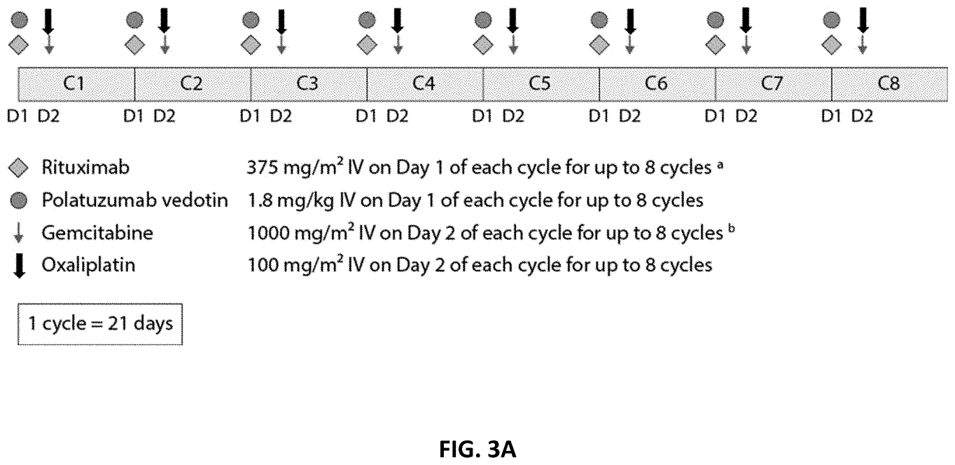

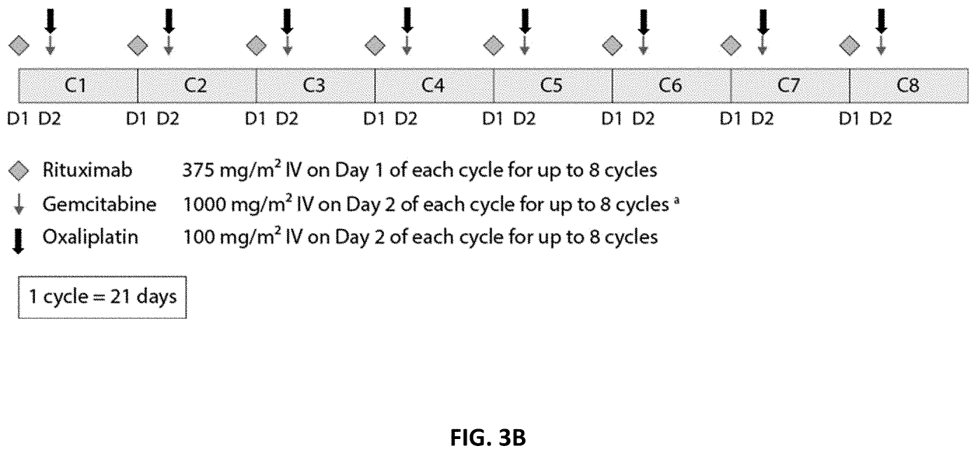

A summary of the Pola-R-GemOx experimental treatment regimen is provided in FIG. 3A.

A summary of the R-GemOx control treatment regimen is provided in FIG. 3B.

### A. Polatuzumab Vedotin

Polatuzumab vedotin 1.8 mg/kg is administered intravenously on Day 1 of each 21-day cycle for up to 8 cycles in the experimental treatment group only (Pola-R-GemOx).

Polatuzumab vedotin is administered after rituximab on the same day or, in the event of a dose delay for rituximab, the following day.

Patient weight obtained during screening (Day −28 to Day −1) is used for dose determination for all treatment cycles; if the patient's weight within 96 hours prior to Day 1 of a given treatment cycle is greater or less than 10% from the weight obtained during screening, the new weight is used to calculate the dose. The weight that triggered a dose adjustment is taken as the new reference weight for future dose adjustments. All subsequent doses are modified accordingly. Dose adjustments for body weight changes <10% are also acceptable should this be local standard of care.

The initial dose is administered to patients who are well hydrated (as per local guidelines) over 90±10 minutes. Premedication (e.g., 500-1000 mg of oral acetaminophen or paracetamol and 50-100 mg diphenhydramine as per institutional standard practice) may be administered to an individual patient ≥30 minutes before starting administration of polatuzumab vedotin. Administration of corticosteroids is permitted at the discretion of the treating physician. If infusion-related reactions (IRRs) are observed with the first infusion in the absence of premedication, premedications are administered before subsequent doses.

The polatuzumab vedotin infusion is slowed or interrupted for patients experiencing infusion-associated symptoms. Following the initial dose, patients are observed for 90 minutes for fever, chills, rigors, hypotension, nausea, or other infusion-associated symptoms. If prior infusions are well tolerated, subsequent doses of polatuzumab vedotin are administered over 30±10 minutes, followed by a 30-minute observation period after the infusion.

During administration of polatuzumab vedotin, vital signs are assessed before the start of the infusion, every 15±5 minutes during the infusion, at the end of the infusion, every 30±10 minutes for 90 minutes following completion of dosing at Cycle 1, and 30±10 minutes following completion of dosing in subsequent cycles.

Polatuzumab vedotin is as a lyophilized formulation (140 mg/vial). Prior to administration, the lyophilized powder is reconstituted with sterile water for injection to a volume of 7.2 mL.

### B. Rituximab

Rituximab (Mabthera/Rituxan®) 375 mg/m² is administered intravenously on Day 1 of each 21-day cycle for up to 8 cycles. Rituximab is administered before polatuzumab vedotin on the same day; in the event of a dose delay for rituximab, polatuzumab vedotin is administered on the following day.

During the administration of rituximab in Cycle 1, vital signs are obtained before infusion, then after the start of the infusion, approximately every 15±5 minutes for 90 minutes, and every 30±10 minutes until 1 hour after the end of the infusion. During administration of rituximab in subsequent cycles, vital signs are recorded before infusion, after the start of infusion, and approximately every 30±10 minutes until 1 hour after the end of infusion.

The patient's body surface area (BSA) calculated at screening is used to calculate the dose of rituximab throughout the study unless the patient's weight increases or decreases by >10% from screening, in which case BSA is recalculated and used for subsequent dosing. In obese patients, there is no BSA cap and actual body weight, not adjusted weight, is recommended. Empiric dose adjustment for obese patients (obesity defined as body mass index ≥30, as measured in kilograms divided by meters squared) is implemented per institutional guidelines.

The rituximab administration is completed at least 30 minutes before administration of other study treatments. The infusion of rituximab is split over 2 days if the patient is at increased risk for an IRR (high tumor burden, high peripheral lymphocyte count). Administration of rituximab is continued on the following day, if needed, for patients who experience an adverse event during the rituximab infusion. If a dose of rituximab is split over 2 days, both infusions occur with appropriate premedication and at the first infusion rate.

All rituximab infusions are administered to patients after premedication with oral acetaminophen (e.g., 650-1000 mg) and an antihistamine such as diphenhydramine hydrochloride (50-100 mg) ≥30 minutes before starting each infusion (unless contraindicated). An additional glucocorticoid (e.g., 100 mg IV prednisone or prednisolone or equivalent) is allowed. For patients who do not experience infusion-related symptoms with their previous infusion, premedication at subsequent infusions may be omitted.

Rituximab is administered as a slow IV infusion through a dedicated line. IV infusion pumps (such as the Braun Infusomat Space) are used to control the infusion rate of rituximab. Administration sets with polyvinyl chloride (PVC), polyurethane (PUR), or polyethylene (PE) as a product contact surface and IV bags with polyolefine, polypropylene (PP), PVC, or PE as a product contact surface are compatible. Additional in-line filters are not used because of potential adsorption. The in-line filter used for the administration of polatuzumab vedotin is not used for the administration of rituximab.

If a patient tolerates the first cycle of study treatment without significant infusion reactions, rituximab is administered as rapid infusion in accordance with local institutional guidelines.

A summary of the administration of the first and subsequent infusions of rituximab is provided in Table 1.

TABLE 1
Administration of first and subsequent infusions of rituximab.

| First Infusion (Cycle 1 Day 1) | Subsequent Infusions |
|---|---|
| Begin infusion at an initial rate of 50 mg/hr | If the patient experiences an |
| If no infusion-related or hypersensitivity | infusion-related or hypersensitivity |
| reaction occurs, increase the infusion rate in | reaction during the prior infusion, begin |
| 50-mg/hr increments every 30 minutes, to a | infusion at an initial rate of 50 mg/hr and |
| maximum of 400 mg/hr. | follow instructions for first infusion. |
| If a reaction develops, stop or slow the | If the patient tolerates the prior infusion |
| infusion. Administer medications and | well (defined by absence of Grade 2 |
| supportive care in accordance with | reactions during a final infusion rate of |
| institutional guidelines. If the reaction | ≥100 mg/hr), begin infusion at a rate of |
| resolves, resume the infusion at a | 100 mg/hr. |
| 50% reduction in rate (i.e., 50% of rate used | If no reaction occurs, increase the infusion |
| at the time the reaction occurred). | rate in 100 mg/hr increments every |
| | 30 minutes, to a maximum of 400 mg/hr. |
| | If a reaction develops, stop or slow the |
| | infusion. Administer medications and |
| | supportive care in accordance with |
| | institutional guidelines. If the reaction |
| | resolves, resume the infusion at a 50% |
| | reduction in rate (i.e., 50% of rate used at |
| | the time the reaction occurred). |

### C. Gemitabine

Gemcitabine 1000 mg/m² is administered intravenously on Day 2 of each 21-day cycle for up to 8 cycles. Gemcitabine is administered before oxaliplatin on the same day. In the event that rituximab dosing is split across two days, gemcitabine is administered on Day 2 (i.e., the same day rituximab dosing is completed) or on the following day.

Cycles are postponed in the event of hematologic toxicity.

The method for administering gemcitabine is described in El Gnaoui et al., (2007) Ann Oncol, 18:1363-1368. In brief, gemcitabine 1000 mg/m² (in 500 mL of normal saline) is administered at a fixed dose rate of 10 mg/m²/min. This prolonged administration schedule has been shown to achieve superior intracellular drug concentrations compared to the standard 30-minute IV schedule.

### D. Oxaliplatin

Oxaliplatin 100 mg/m² is administered intravenously on Day 2 of each 21-day cycle for up to 8 cycles. Oxaliplatin is administered after gemcitabine on the same day. In the event that rituximab dosing is split across two days, oxaliplatin is administered on Day 2 (i.e., the same day rituximab dosing is completed) or on the following day.

The method for administering oxaliplatin is described in local prescribing information.

### E. Dose Modifications

Dose modifications or reductions are carried out as set forth in Table 2. All adverse events are based on laboratory test results obtained within 72 hours before infusion of Day 1 of that cycle. Grading of symptoms is according to NCI-CTCAE v5.0. For supportive treatment, patients are treated with acetaminophen/paracetamol and an antihistamine such as diphenhydramine if they have not been received in the previous 4 hours. IV saline may be indicated. For bronchospasm, urticaria, or dyspnea, patients may require antihistamines, oxygen, corticosteroids (e.g., 100 mg IV prednisolone or equivalent), and/or bronchodilators. Patients with hypotension who require vasopressor support are permanently discontinued from study drug. For infusion rate escalation after re-initiation, upon complete resolution of symptoms, the infusion is resumed at 50% of the rate achieved prior to interruption. In the absence of infusion-related symptoms, the rate of infusion is escalated in increments of 50 mg/hour every 30 minutes.

TABLE 2
Guidelines for management of patients who experience adverse events.

| Event | Dose Delay or Modification |
|---|---|
| Grade 3 or 4 neutropenia | All treatment is held until ANC recovers to >1000/μL. |
| | Growth factors are administered if necessary (e.g., G-CSF). |
| | All treatment without any additional dose reductions is resumed |
| | if ANC recovers to >1000/μL on or before Day 7. |
| | The dose of polatuzumab vedotin is reduced to 1.4 mg/kg if |
| | after Day 7. If a prior dose reduction of polatuzumab vedotin has |
| | occurred, then treatment is discontinued. |
| Grade 3 or 4 | All treatment is held until platelets recover to >75,000/μL. |
| thrombocytopenia | All treatment is resumed without any additional dose reductions |
| | if platelets recover to >75,000/μL on or before Day 7. |
| | The dose of polatuzumab vedotin is reduced to 1.4 mg/kg if |
| | platelets recover to >75,000/μL after Day 7. If a prior dose |
| | reduction of polatuzumab vedotin has occurred, then treatment is |
| | discontinued. |
| Grade 2 or 3 peripheral | All study treatments are delayed until improvement to ≤Grade 1. |
| neuropathy | Study treatment is restarted with polatuzumab vedotin at a |
| | permanently reduced dose of 1.4 mg/kg and oxaliplatin at a dose |
| | of 75 mg/m² if recovered to Grade ≤1 within ≤14 days. |
| | Polatuzumab vedotin alone is discontinued if not recovered to |
| | Grade ≤1 on or before Day 14 if the patient previously had |
| | Grade 2 peripheral neuropathy and/or if a prior dose reduction to |
| | 1.4 mg/kg for polatuzumab vedotin or 75 mg/m² for oxaliplatin |
| | has occurred. Study treatment is discontinued if the patient |
| | previously had Grade 3 peripheral neuropathy. |
| | Oxaliplatin and polatuzumab vedotin are permanently |
| | discontinued if not recovered to ≤Grade 1 until >14 days or |
| | after the scheduled date for the next cycle. |
| Grade 4 peripheral | All treatment is discontinued. |
| neuropathy (including its | |
| signs and symptoms) | |
| Pharyngolaryngeal | Oxaliplatin infusion is prolonged to 6 hours. |
| dysesthesia | |
| Grade 1-2 IRR | Infusion is slowed or held. |
| | Patients are given supportive treatment. |
| | Upon symptom resolution, infusion-rate escalation is resumed. |
| | For Grade 1-2 IRR, polatuzumab vedotin is infused over 90 |
| | minutes in the next cycle. If no infusion-related reaction occurs, |
| | subsequent infusions are administered over 30 minutes. |
| | Premedication is administered for all cycles |
| | For Grade 2 wheezing or urticaria, if symptoms recur, the |
| | infusion is stopped immediately and study drug is permanently |
| | discontinued. |
| Grade 3 IRR | Infusion is discontinued. |
| | Supportive treatment is given. |
| | Upon symptom resolution, infusion-rate escalation is resumed. |
| | If the same adverse event recurs with same severity, treatment is |
| | permanently discontinued. |
| | If patient has Grade 3 wheezing, bronchospasm, or generalized |
| | urticaria at first occurrence, study drug is permanently |
| | discontinued. |
| Grade 4 IRR | Infusion is discontinued immediately, symptoms are treated |
| | aggressively, and study drug is permanently discontinued. |
| Total bilirubin >3.0 mg/dL | Treatment is delayed until resolution to ≤1.5 mg/dL within ≤14 |
| | days. Causality is evaluated. |
| | Any case involving an increase in hepatic transaminase >3 × |
| | baseline and an increase in direct bilirubin >2 × ULN, without |
| | any findings of cholestasis or jaundice or signs of hepatic |
| | dysfunction and in the absence of other contributory factors (e.g. |
| | worsening of metastatic disease or concomitant exposure to |
| | known hepatotoxic agent or of a documented infectious etiology) |
| | suggests potential drug-induced liver injury (DILI), and study |
| | treatment is discontinued. |
| Grade 3 or 4 tumor lysis | All study treatment is held. The patient's next dose is delayed for |
| syndrome | up to 14 days. |
| | Following complete resolution of TLS, study treatment is re- |
| | administered at the full dose during next scheduled infusion in |
| | conjunction with prophylactic therapy. |
| Grade 3 or 4 non-hematologic | Study treatment is delayed for a maximum of 14 days. |
| toxicity not specifically | Study therapy is continued at a reduced dose or at the full |
| described above (excluding | dose if improvement to Grade ≤1 or baseline is observed. |
| alopecia, nausea, and | |
| vomiting) | |
| Grade 2 non-hematologic | Study treatment is delayed for a maximum of 14 days |
| toxicity | Previous doses of study treatment are administered if |
| | improvement to Grade ≤1 or baseline is observed. |
| Grade 1 non-hematologic | No dose reduction or delay. |
| toxicity | |
| Hepatitis B reactivation (as | HBV-DNA levels are retested within 2 weeks if HBV-DNA |
| noted by new detectable | levels are between the WHO-recommended range of 29 and 100 |
| HBV-DNA levels) | IU/mL. If still positive, all study treatment is held and patient is |
| | treated with an appropriate nucleoside analogue. Patient is |
| | referred to a gastroenterologist or hepatologist. |
| | If HBV-DNA levels are at the WHO-recommended cutoff of >100 |
| | IU/mL all study treatment is held and patient is treated with |
| | an appropriate nucleoside analogue. Patient is referred to a |
| | gastroenterologist or hepatologist. |
| | If rising HBV-DNA viral load (exceeding 100 IU/mL) while on |
| | an appropriate anti-viral therapy is observed, all study treatment |
| | is discontinued. |

ANC = absolute neutrophil count; G-CSF = granulocyte colony stimulating factor; HBV = hepatitis B virus; IRR = infusion-related reaction; IV = intravenous; LMWH = low molecular-weight heparin;
R = rituximab; ULN = upper limit of normal; WHO = World Health Organization.

### F. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study treatment to the study completion/discontinuation visit. All such medications are recorded.

Patients are permitted to use the following therapies during the study: oral contraceptives, hormone-replacement therapy, and/or other maintenance therapies.

In general, investigators manage a patient's care with supportive therapies as clinically indicated, per local standard practice. Necessary supportive measures for optimal medical care are given throughout the study according to institutional standards, including the use of growth factors (e.g., erythropoietin), if clinically indicated. Anti-emetic therapy is instituted for any patient if clinically indicated. Herbal therapies not intended for the treatment of cancer are used during the study at the discretion of the investigator.

### G. Premedications

Premedication Before Polatuzumab Vedotin

Polatuzumab vedotin infusions immediately follow rituximab infusions. Consequently, as premedication is required before all rituximab infusions (see below), additional premedication for polatuzumab vedotin is not required.

However, if there is a delay in dosing of polatuzumab vedotin, then polatuzumab vedotin is administered following premedication. Premedication consists of 500-1000 mg of oral acetaminophen or paracetamol and 50-100 mg diphenhydramine (as per institutional standard practice) administered ≥30 minutes before starting administration of polatuzumab vedotin. Administration of corticosteroids is permitted at the discretion of the treating physician.

Premedication Before Rituximab

All rituximab infusions are administered to patients after premedication. The following premedication is required before rituximab therapy:
Acetaminophen/paracetamol (650-1000 mg) orally at ≥30 minutes before the start of all infusions.
Antihistamine, such as diphenhydramine (25-50 mg), ≥30 minutes before the start of each infusion (unless contraindicated).

Premedication for Patients with High Risk of Tumor Lysis Syndrome

Patients with high tumor burden and considered by the investigator to be at risk for tumor lysis also receive tumor lysis prophylaxis prior to the initiation of treatment.

Patients are well hydrated. Starting at 1-2 days prior to the first dose of study treatment, it is desirable to maintain a fluid intake of approximately 3 L/day.

In addition, patients with high tumor burden and considered to be at risk for tumor lysis are treated with 300 mg/day of allopurinol orally or a suitable alternative treatment (e.g., rasburicase), starting 48-72 hours prior to Cycle 1 Day 1 of treatment and hydration. Patients continue to receive repeated prophylaxis if deemed appropriate by the investigator, along with adequate hydration prior to each subsequent cycle of treatment.

Prophylaxis for Infections

Anti-infective prophylaxis for pneumocystis and herpesvirus infections is instituted per institutional practice or investigator preference based on individual patient risk factors. Patients are treated with prophylactic anti-viral medications for hepatitis B reactivation in countries where such medications are the standard of care (Flowers et al., 2013; NCCN 2017).

Prophylaxis and Treatment of Neutropenia

The administration of G-CSF as a primary prophylaxis in each cycle of therapy is administered as per local/institutional guidelines. The dose and form of G-CSF is at the discretion of the investigator. The use of additional G-CSF is allowed for the treatment of neutropenia per investigator discretion.

Monitoring and Treatment for Hepatitis B Reactivation

Patients with occult or prior HBV infection (defined as negative HBsAg and positive hepatitis B core antibody [HBcAb]) are included in this study if HBV DNA is undetectable. These patients have HBV DNA levels obtained on Day 1 of every cycle and for at least 12 months after the last cycle of study treatment by means of real-time PCR, with the use of an assay that has a sensitivity of at least 10 IU/mL

If the HBV-DNA assay becomes positive and is above the World Health Organization's (WHO) cutoff of 100 IU/mL, study treatment is held and the patient is treated (for at least 1 year after the last dose of rituximab) with an appropriate nucleoside analogue and immediately referred to a gastroenterologist or hepatologist for management. Patients resume study treatment once HBV DNA levels decrease to undetectable levels.

If the HBV DNA assay becomes positive and is ≤100 IU/mL, the patient is retested within 2 weeks. If the assay is still positive, study treatment is held, and the patient is treated with an appropriate nucleoside analogue (for at least 1 year after the last dose of rituximab) and immediately referred to a gastroenterologist or hepatologist for management. Patients resume study treatment once the HBV DNA levels decrease to undetectable levels.

If a patient's HBV DNA level exceeds 100 IU/mL while the patient is receiving anti-viral medication, study treatment is permanently discontinued.

Patients in countries where prophylactic anti-viral medications for hepatitis B reactivation are the standard of care are treated prophylactically.

Cautionary Therapy

Caution is exercised in the administration of oxaliplatin in patients with a history or a predisposition for prolongation of QT, those who are taking medicinal products known to prolong QT interval, and those with electrolyte disturbances such as hypokalemia, hypocalcemia, or hypomagnesemia. The QT interval is closely monitored on a regular basis before and after administration of oxaliplatin. ECG recordings are obtained at specified timepoints and as clinically indicated. In case of QT prolongation, oxaliplatin treatment is discontinued.

Patients who are receiving strong CYP3A inhibitors are closely monitored for adverse reactions when given polatuzumab vedotin (Han et al., (2013) J Clin Pharmacol, 53:866-77).

Concomitant medications that are β-gp inhibitors are considered cautionary, as they may potentially lead to adverse reactions that require close monitoring. If a patient is taking any of the medications in the categories of β-gp inhibitors, the investigator assesses and documents the use of medications known or suspected to fall in those categories. Patients receiving strong β-gp inhibitors concomitantly with vc-MMAE ADCs (e.g., polatuzumab vedotin) are closely monitored for adverse reactions.

Prohibited Therapy

Treatment with other concomitant anti-tumor agents not defined in this protocol as study treatment, radiotherapy, or other concurrent investigational agents of any type result in withdrawal of patients from study treatment.

Use of the following concomitant therapies is prohibited as described below:
Investigational therapy (other than protocol-mandated study treatment) is prohibited within 2 weeks prior to initiation of study treatment and during study treatment.
Cytotoxic chemotherapy, other than gemcitabine, oxaliplatin, and intrathecal chemotherapy for CNS prophylaxis.
Immunotherapy or immunosuppressive therapy, other than study treatments.
Radioimmunotherapy.
Hormone therapy, other than contraceptives, stable hormone-replacement therapy, or megestrol acetate.
Biologic agents for the treatment of lymphoma.
   Biological agents that are supportive therapies, such as hematopoietic growth factors, are allowed if clinically indicated.
Any therapy (other than intrathecal CNS prophylaxis) intended for the treatment of lymphoma.
Radiotherapy.
Immunizations.
   Patients who participate in this study may not receive either primary or booster vaccinations with live virus vaccines for at least 28 days before initiation of rituximab, at any time during study treatment, or until B-cell recovery.

Patients who require the use of any of these agents are discontinued from study treatment.

### V. Study Assessments

### A. Patient and Disease Characteristics

Medical history, including clinically significant diseases, surgeries, cancer history (including prior cancer therapies, reason for transplant ineligibility, 2016 WHO classification, current Ann Arbor stage, and procedures), ECOG performance status, and reproductive status are recorded. In addition, all medications (e.g., prescription drugs, over-the-counter drugs, vaccines, herbal or homeopathic remedies, nutritional supplements) used by the patient within 7 days prior to initiation of study treatment are recorded.

A complete physical examination is performed and any abnormality identified at baseline is recorded.

As part of tumor assessment, physical examinations include evaluation of the presence and degree of enlarged lymph nodes, hepatomegaly, and splenomegaly.

During the study, limited, symptom-directed physical examinations are performed and are limited to systems of primary relevance (i.e., cardiovascular, respiratory, those associated with symptoms, and those associated with tumor assessment [lymph nodes, liver, and spleen]). Limited physical exams also monitor for symptoms of neuropathy, including hypoesthesia, hyperesthesia, paresthesia, dysesthesia, discomfort, a burning sensation, weakness, gait disturbance, loss of balance, orthostatic hypotension, syncope, or neuropathic pain. Changes from baseline abnormalities are recorded.

Vital signs, weight, height, and BSA are recorded. Height and BSA are required at screening only, unless there is a >10% change in body weight since the last BSA assessment, in which case BSA is recalculated. ECG recordings are obtained.

Comparison of the experimental treatment and control arm groups is performed.

Demographic variables such as age, sex, race/ethnicity, and baseline characteristics (in particular, stratification variables) are summarized by treatment arm for all randomized patients. Continuous variables are summarized with use of means, standard deviations, medians, ranges, and inter-quartile ranges. Categorical variables are summarized by proportions.

Descriptive statistics are presented for cumulative study medication doses, dose modifications/interruptions, and duration of exposure. ECGs are analyzed descriptively. Changes in vital signs are analyzed using descriptive statistics for continuous variables.

The following assessments are performed:
The International Prognostic Index (IPI): a clinician-assessed tool that is prognostic of OS in patients with NHL (International Non-Hodgkin's Lymphoma Prognostic Factors 1993). The instrument is based on measurements of five clinical factors, including age, serum and lactate dehydrogenase (LDH) level, ECOG performance status, cancer stage and extra-nodal site involvement.
The Eastern Cooperative Oncology Group Performance (ECOG): a clinician-assessed tool that describes a patient's level of functioning in terms of their ability to care for themselves, daily activity, and physical ability (walking, working, etc.) (ECOG ACRIN Cancer Research Group 2018).

The following laboratory tests are performed:
Hematology: white blood cell (WBC) count, red blood cell (RBC) count, hemoglobin, hematocrit, platelet count, differential count (neutrophils, eosinophils, basophils, monocytes, lymphocytes).
Serum or plasma chemistry: sodium, potassium, chloride, bicarbonate (or CO₂), glucose, blood urea nitrogen (BUN) or urea, creatinine, total protein, albumin, phosphorus, calcium, total and (if available) direct bilirubin, alkaline phosphatase, alanine aminotransferase (ALT), aspartate aminotransferase (AST), uric acid, and lactate dehydrogenase (LDH).
Coagulation: international normalized ratio (INR) or prothrombin time (PT), and partial thromboplastin time (PTT) or activated partial thromboplastin time (aPTT).
Viral serology: HIV; hepatitis B surface antibody (HBsAb), HBsAg, and total HBcAb; HBV-DNA by PCR if the patient is HBcAb positive; HCV antibody; HCV RNA by PCR if the patient is HCV antibody positive.

Laboratory data with values outside the normal ranges are. In addition, changes in selected laboratory data and vital signs are summarized.

### B. Statistical Assessments

The analysis populations are defined as follows:
Intent to Treat (ITT) population: all randomized patients, grouped according to their assigned treatment arm, whether or not the assigned study treatment is received.
Safety run-in population: patients who receive any amount of any study drug during safety run-in phase (Stage 1).
Safety population: patients who receive any amount of any study drug during the randomized controlled trial (Stage 2).
PRO-evaluable population: PRO-evaluable population includes all randomized patients who have a baseline and at least 1 post-baseline assessment. All PRO analyses are performed based on the treatment arm assigned at randomization.
Pharmacokinetic-Evaluable Population: The PK population includes all patients who receive at least one dose of study drug and have at least one post-dose concentration result.

For all efficacy analyses, patients are grouped according to the treatment assigned at randomization. For all safety analyses, patients are grouped according to the treatment actually received.

Hypothesis tests are two-sided, unless otherwise indicated. The type I error (u) for this study is 0.05 (two-sided).

### C. Safety

Safety is assessed by adverse events per NCI CTCAE v5.0, FACT/GOG-Ntx12 score, clinical laboratory test results, electrocardiogram (ECG) and vital signs.

Safety assessments include monitoring and recording adverse events, including serious adverse events and adverse events of special interest, performing safety laboratory assessments, measuring vital signs, and conducting other tests that are deemed critical to the safety evaluation of the study. After initiation of study drug, all adverse events are reported until 90 days or the initiation of NALT after the last dose of study drug.

Safety is assessed through summaries of adverse events and study treatment exposures and are presented by treatment arm.

Verbatim descriptions of adverse events are summarized by mapped term, appropriate thesaurus level, and toxicity grade. For each patient, if multiple incidences of the same adverse events occur, the maximum severity reported is used in the summaries.

The following treatment-emergent adverse events are summarized separately: adverse events leading to withdrawal of study drug, adverse events leading to dose reduction or interruption, Grade ≥3 adverse events, adverse events leading to death, serious adverse events, and adverse events of special interest. In addition, exposure-adjusted analyses, as well as analysis of recurrent AEs, are provided as appropriate.

All deaths and causes of death are summarized.

Relevant laboratory values are summarized by time, with NCI CTCAE v5.0 Grade 3 and Grade 4 values identified, where appropriate. Changes in NCI CTCAE grade are tabulated by treatment arm.

Throughout the study, peripheral neuropathy, as measured by FACT/GOG-Ntx12 score and by NCI CTCAE v5.0, is summarized. In addition, the rate of peripheral neuropathy based on AE is calculated, including all patients complete Cycle 2 or report this type of event before.

Patient-reported outcomes are analyzed by examining and summarizing time to deterioration, as well as change from baseline for selected questions of the different PRO instruments.

Descriptive statistics are presented for cumulative study medication doses, dose modifications/interruptions, and duration of exposure. ECGs are analyzed descriptively. Changes in vital signs are analyzed using descriptive statistics for continuous variables.

Adverse events of special interest for this study are as follows:
Peripheral neuropathy ≥Grade 3.
Cases of potential drug-induced liver injury that include an elevated ALT or AST in combination with either an elevated bilirubin or clinical jaundice, as defined by Hy's Law. The following are reported as adverse events:
   Treatment-emergent ALT or AST >3×baseline value in combination with total bilirubin >2×ULN (of which >35% is direct bilirubin).
   Treatment-emergent ALT or AST >3×baseline value in combination with clinical jaundice.
Tumor lysis syndrome of any grade (irrespective of causality).
Progressive multifocal leukoencephalopathy.
Systemic hypersensitivity reactions/anaphylactic and anaphylactoid reactions, as defined by Sampson's criteria.
Second malignancies.

Events that are clearly consistent with the expected pattern of progression of the underlying disease are not recorded as adverse events. Adverse event reports are not derived from PRO data.

The investigator follows each adverse event until the event resolves to baseline grade or better, the event is assessed as stable by the investigator, the patient is lost to follow-up, or the patient withdraws consent.

After the end of the adverse event reporting period (defined as 90 days after the last dose of study drug or the initiation of NALT), all deaths regardless of cause, and serious AEs believed to be related to prior exposure to study drug are reported.

All patients with AEs of peripheral neuropathy are followed up after discontinuation of study treatment for possible deterioration (Coasting phenomenon) and thereafter until resolution or stabilization.

Dose interruptions, dose reductions and dose intensity are used to determine tolerability.

### D. Efficacy

Primary Efficacy Endpoint

The primary efficacy objective for the randomized part of this study is to evaluate the efficacy of Pola-R-GemOx compared with R-GemOx in patients with relapsed or refractory DLBCL on the basis of the following endpoint:
Overall survival (OS), defined as the time from randomization to the death from any cause during the study.

The primary efficacy analysis is completed on the ITT population, with patients grouped according to their treatment assigned at randomization. Data for patients without death is censored at the date at which the patient was last known to be alive. Otherwise, data is censored at the date of randomization+1 day.

Kaplan-Meier methodology is used to estimate the median OS for each treatment arm, and Kaplan-Meier curves are produced. OS is compared between treatment arms by stratified logrank test. The hazard ratio (HR) for death is estimated using a stratified Cox proportional hazards model. The stratification factors are the same as the randomization stratification factors by IxRS, as described above. The 95% confidence interval (CI) for the HR is provided. Brookmeyer-Crowley methodology is used to construct the 95% CI for the median OS for each treatment arm.

The type I error (u) for this study is 0.05 (two-sided).

In case patients become eligible for HSCT and undergo a transplant (in their best interest), sensitivity analyses are performed for OS and PFS to assess the impact this may have. For this, patient data is censored at the time of transplant.

Secondary Efficacy Endpoints

The following sections detail the analyses for the secondary endpoints of the randomized part of the study (Stage 2). Similar analyses are performed for the safety run-phase (Stage 1), but are restricted to descriptive statistics.

PET-CT and CT scans are obtained at screening, as clinically indicated during and after treatment, 28 days after the last dose of study drug and then every two (PET-CT) and six (CT) months during follow up for up to two years.

To control the overall type I error rate at a two-sided 0.05 level of significance, a hierarchical testing procedure is used to adjust for multiple statistical testing of the primary and key secondary efficacy endpoints. Key secondary endpoints is tested in the following order:
PFS.
Complete response rate (CRR) at end of treatment (based on response including PET-CT data).
Objective response rate (ORR) at end of treatment (based on response including PET-CT data).

A given hypothesis is only rejected once all previous hypotheses have been rejected at a 2-sided 0.05 level of significance.

No multiplicity adjustment is performed for the testing of other endpoints and is interpreted with caution.

Response is assessed on the basis of physical examinations, and PET-CT scans using Lugano 2014 Response Criteria at the end of treatment. Tumor assessments occur at Screening, mid-treatment (From Cycle 4, Day 15 to Cycle 5, day 1) and at the end of treatment. The initial and end-of-treatment assessments include PET. CT scans are completed every 6 months for 2 years in the long-term follow up or until the end of the study. All primary imaging data used for tumor assessments is collected.

PET-CT scans include skull-base to mid-thigh. Full-body PET-CT scans are performed when clinically appropriate. Image enhancement systems (such as GE Healthcare's Q.Clear or similar) are not used. CT scans with IV contrast include chest, abdomen, and pelvic scans; CT scans of the neck are included if clinically indicated. CT scans for response assessment are limited to areas of prior involvement only if required by local regulatory authorities. At the investigator's discretion, CT scans are repeated at any time if progressive disease is suspected. A full tumor assessment including radiographic assessment is performed any time disease progression or relapse is suspected. PET-CT is mandatory at the screening and end-of-treatment assessments.

Bone marrow biopsies are performed in patients with negative bone signal on PET-CT.

In patients for whom contrast is contraindicated, (e.g., patients with contrast allergy or impaired renal clearance), CT or combined PET-CT scans without contrast are permitted so long as they permit consistent and precise measurement of target lesions during the study treatment period.

Patients with a response of PR or CR for whom subsequent therapy is indicated (e.g., CAR-T therapy or autologous stem cell transplantation) continue on study and remain evaluable.

Complete Response Rate

The complete response rate (CRR) is defined as the proportion of patients who have a complete metabolic response (based on response including PET-CT data) according to Lugano 2014 response criteria at the end of treatment. Patients not meeting these criteria, including patients without any post-baseline tumor assessment, are considered non-responders.

CRR is analyzed using the ITT population.

An estimate of CRR is calculated for each treatment arm, and its 95% confidence interval (CI) is calculated using the Clopper-Pearson method. The difference in CRR between treatment arms is calculated, and its 95% CI is calculated using the normal approximation to the binomial distribution. CRR is compared between treatment arms using the stratified Cochran-Mantel-Haenszel test. The stratification factors are the same as those described for the analysis of the primary endpoint OS.

The same analysis is repeated for the CRR using the response not including PET data, and therefore considering patients who have complete response instead of complete metabolic response.

Objective Response Rate

An objective response is defined as either a complete or a partial metabolic response (based on response including PET-CT data) according to Lugano 2014 response criteria at end of treatment. Patients not meeting these criteria, including patients without any post-baseline tumor assessment, are considered non-responders.

ORR is defined as the proportion of patients who have an objective response.

ORR is analyzed using the ITT population.

An estimate of ORR is calculated for each treatment arm, and its 95% CI is calculated using the Clopper-Pearson method. The difference in ORR between treatment arms is calculated, and its 95% CI is calculated using the normal approximation to the binomial distribution. ORR is compared between treatment arms using the stratified Cochran-Mantel-Haenszel test. The stratification factors are the same as those described for the analysis of the primary endpoint OS.

The same analysis is repeated for the ORR using the response not including PET data, objective response (based on response not including PET data) being then defined as either a complete or partial response (based on response not including PET data) at end of treatment.

Best Overall Response

Best overall response (BOR) is defined as the best response while on study (based on response including PET-CT or CT data) according to Lugano 2014 response criteria.

BOR is analyzed using the ITT population.

An estimate of BOR rates is calculated for each treatment arm, and its 95% CI is calculated using the Clopper-Pearson method.

Progression-Free Survival

Progression-free survival (PFS) is defined as the time from randomization to the first occurrence of disease progression, or death due to any cause, whichever occurs first. Patients who die without a reported disease progression are considered as an event on the date of death. Patients who have neither progressed nor died at the time of analysis (clinical-cut off) and patients who are lost to follow-up are censored on the date of the last evaluable tumor assessment. Patients who do not undergo a post-baseline tumor assessment are censored at the time of randomization+1 day. Kaplan-Meier estimates and the associated 95% CIs of the median, 25th and 75th percentile are presented. The Kaplan-Meier curve provides a visual description of the differences across treatment arms. Estimates of the treatment effect are expressed as hazard ratios, using a stratified Cox proportional-hazards analysis including 95% confidence limits.

Duration of Response

Duration of response (DOR) is assessed in patients who have an objective response using Lugano 2014 response criteria. DOR is defined as the time interval from the date of the first occurrence of a complete or partial response (whichever status is recorded first) until the first date that progressive disease or death is documented, whichever occurs first. Patients who have not progressed and who have not died at the time of analysis are censored at the time of last tumor assessment date. If no tumor assessments are performed after the date of the first occurrence of a complete or partial response, DOR is censored at the date of the first occurrence of a complete or partial response plus 1 day. DOR is based on a non-randomized subset of patients (specifically, patients who achieve an objective response), therefore, comparisons between treatment arms is made for descriptive purposes. The methodologies detailed for the PFS analysis are used for the DOR analysis, except that the analysis is not stratified.

Event-Free Survival

Event free survival (EFS_{eff}) is defined as the time from randomization to the earliest occurrence of the below cases:
Disease progression or relapse.
Death due to any cause.
Initiation of any NALT.

Patients with no EFS_{eff} events are censored at the time of the last evaluable tumor assessment. Patients who do not undergo a post-baseline tumor assessment are censored at the time of randomization. The methodologies detailed for the PFS analysis are used for the EFS_{eff} analysis.

### E. Patient-Reported Outcomes

Patient-reported outcome (PRO) instruments are completed to assess the treatment benefit and more fully characterize the safety profile of polatuzumab vedotin. In addition, PRO instruments enable the capture of each patient's direct experience with polatuzumab vedotin.

The PRO-evaluable population is used for descriptive analyses of visit summaries and change from baseline, responder analyses, and mixed-effects model repeated measures (MMRM) modeling, unless specified otherwise. The ITT population is used for completion analyses and time-to-deterioration analyses.

PRO data is collected through use of the following instruments: FACT/GOG-Ntx12, EQ-5D-5L, EORTC QLQ-C30 and FACT/Lym.

FACT/GOG-Ntx12

The FACT/GOG-Ntx12 is a 12-item patient-reported outcome instrument that was designed to measure chemotherapy-induced peripheral neuropathy (Kopec et al., (2006) J Supportive Oncol, 4:W1-W8). FACT/GOG-Ntx scores are reported throughout the trial, including during Stage 1 (Safety Run-In) and Stage 2 (RCT). For each of the FACT/GOG-Ntx-12 questionnaire scales, descriptive statistics at each visit and changes from baseline are reported by treatment arm. For missing items within the questionnaire, prorated scores are calculated according to developer guidance (Calhoun et al., (2003) Int J Gynecol Cancer, 13:741-748). PRO completion rates are summarized at each time point by treatment arm. For neurotoxicity subscale, descriptive statistics at each visit and changes from baseline are reported on the safety run-in population.

EO-5D-5L

The EuroQol 5-Dimension Questionnaire, [5-level version (EQ-5D-5L]), is a validated self-report health status questionnaire that is used to calculate a health status utility score suing 5 dimensions (EuroQol (1990) Health Policy, 16:199-208; Brooks (1996) Health Policy, 37:53-72; Herdman et al., (2011) 20:1727-1736; Janssen et al., (2013) Qual Life Res, 22:1717-1727). All five dimensions can be combined in a five-digit number that describes the patient's health state. This descriptive number is converted to a single summary index utility score by using published weights. In this study, the UK crosswalk value set (as published by the EuroQol Research Foundation at http://www(dot)euroqol(dot)org/about-eq-5d/valuation-of-eq-5d; Devlin et al., (2017) Health Economics, 1-16) is used. Additionally, in the second part of the questionnaire, the current health status is measured by the visual analog scale (VAS) with values ranging from 0 to 100.

For each of the EQ-5D-5L assessments over time, the number and percentage of patients in each of the five categories for each question is evaluated. A summary of the EQ-5D-5L index utility score at each visit and the associated change from baseline is provided by treatment arm. A similar analysis is performed for EuroQoL visual analog scale (EQ-VAS).

The index utility score and VAS are analyzed by mixed linear models. In addition, the proportion of patients who report changes that exceed the clinically meaningful threshold on the EQ-5D-5L index and the EQ-VAS scores are reported. A clinically meaningful improvement threshold is defined as a change of +0.07 points in the index utility score and +7 points in the VAS score.

EORTC OLO-C30

The EORTC QLQ-C30 is a validated, reliable self-report measure (Aaronson et al., (1993) J Natl Cancer Inst, 85:365-376; Fitzsimmons et al., (1999) 35:939-941). It consists of 30 questions that assess five aspects of patient functioning (physical, emotional, role, cognitive, and social), three symptom scales (fatigue, nausea and vomiting, pain), global health/quality of life, and six single items (dyspnea, insomnia, appetite loss, constipation, diarrhea, and financial difficulties) with a recall period of the previous week.

For EORTC QLQ-C30 questionnaire, summary statistics at each visit and change from baseline of linear-transformed scores are reported for all the items and the subscales.

Time to deterioration is defined as the time from randomization to the first documentation of a 10-point decrease in EORTC QLQ-C30 physical functioning scale from baseline. For fatigue, time to deterioration is defined as the time from randomization to the first documentation of a 10-point increase from baseline. Patients who do not have an observed deterioration at the time of clinical data cut-off are censored at the last non-missing assessment date if post-baseline assessment, or on the date of randomization+1 day if no post-baseline assessment. The hazard ratio for time to deterioration is estimated using a stratified Cox proportional hazards model. The 95% CI for the hazard ratio is provided. Kaplan-Meier methodology is used to estimate the median time to deterioration for each treatment arm, and Kaplan-Meier curves are produced.

The EORTC QLQ-C30 data are scored according to the EORTC scoring manual (Fayers et al., (2001) European Organisation for Research and Treatment of Cancer, Brussels). Missing data is assessed and reported by time point. In the event of incomplete data, for all questionnaire subscales, if more than 50% of the constituent items are completed, a pro-rated score is computed consistent with the scoring manuals and published validation reports. For subscales with less than 50% of the items completed, the subscale is considered as missing. Completion rates are summarized by number and proportion of patients among those expected to complete the EORTC QLQ-C30 at each time point.

FACT-Lym

The FACT-Lym is a validated health-related quality of life (HRQoL) instrument used specifically in patients with lymphoma. It is composed of FACT-general questionnaire (FACT-G), plus the Lymphoma-Specific Subscale (FACT-Lym LYMS; range 0-60) (Cella et al., (1993) J Clin ONcol, 11:570-579; Cella et al., (2005) Blood, 106:750). Three summary scales, the FACT-Lym trial outcome index, the FACT-G, and the FACT-Lym total score are calculated. Higher scores are reflective of better HRQoL.

For each of the FACT-Lym questionnaire scales, descriptive statistics at each visit and changes from baseline are reported by treatment arm.

Clinically meaningful minimally important differences (i.e., the smallest amount of change considered important to patients) at the individual subscale and FACT-Lym TOT level are pre-specified and are used to define the proportion of patients reporting meaningful changes on the FACT-Lym LYMS (≥3 points), FACT-Lym TOI (≥6 points), and FACT-Lym TOT (≥7 points) scales as a result of treatment (Carter et al., (2008) Blood, 112:2376).

Time to deterioration is defined as the time from randomization to the first documentation of a >3-point decrease from baseline (Carter et al., (2008) Blood, 112: 2376; Hlubocky et al., (2013) Lymphoma, ID147176). Patients who do not have an observed deterioration at the time of clinical data cut-off are censored at the last non-missing assessment date if post-baseline assessment, or on the date of randomization+1 day if no post-baseline assessment. The hazard ratio for time to deterioration is estimated using a stratified Cox proportional hazards model. The 95% CI for the hazard ratio is provided. Kaplan-Meier methodology is used to estimate the median time to deterioration for each treatment arm, and Kaplan-Meier curves are produced. Supplemental item-level analyses are conducted with the individual B-symptom items of the FACT-Lym LYMS using a raw 1-point worsening. For missing items within the questionnaire, prorated scores are calculated according to developer guidance (Webster et al., (2003) Health Qual Life Outcomes, 1:79). PRO completion rates are summarized at each time point by treatment arm.

### F. Pharmacokinetic Analyses

During Stage 1 and Stage 2, PK analyses of polatuzumab vedotin are performed on blood samples. Serum polatuzumab vedotin total antibody (which includes all drug-to-antibody ratio [DAR] species, including DAR 0 and DAR ≥1), plasma polatuzumab vedotin conjugate (evaluated as acMMAE), and plasma unconjugated MMAE concentrations are quantified with the use of validated methods. Samples are also analyzed for other potential catabolites.

Individual and mean serum and plasma concentrations of polatuzumab vedotin, gemcitabine, and oxaliplatin versus time data are tabulated and plotted. Summary statistics of concentration data are computed for each sampling for each analyte. PK parameters, maximum concentrations (Cₘₐₓ), and trough concentration (Cₜro_{ugh}), are estimated (as appropriate for the data collected). Estimates for these parameters are tabulated and summarized (mean and SD). PK parameters are determined using the appropriate technique based on available data. Population PK analysis method is applied for PK parameter estimation. Potential drug interactions are assessed by comparison of PK in the current study with historical data. Potential correlations between PK variability and demographic and pathophysiological covariates are explored by population PK analysis. Potential correlations between PK variability and pharmacodynamic, efficacy, and safety outcome are explored by exploratory graphical analysis and PK-pharmacodynamic modeling.

### G. Immunogenicity Analyses

During Stage 1 and Stage 2, a validated antibody-bridging ELISA is used to screen for and confirm the presence of anti-polatuzumab vedotin antibodies in patient serum samples, as well as to characterize and determine the titer of confirmed ADA-positive samples.

The immunogenicity analysis population consists of all patients who receive at least one dose of polatuzumab vedotin with at least one evaluable post-baseline ADA sample. Patients are grouped according to treatment received or, if no treatment is received prior to study discontinuation, according to treatment assigned.

The numbers and proportions of ADA-positive and ADA-negative patients at baseline (baseline prevalence) and after baseline (post-baseline incidence) are summarized by treatment group. When determining post-baseline incidence, patients are considered to be ADA positive if they are ADA negative or have missing data at baseline but are tested positive for ADAs following study drug exposure (treatment-induced ADA response); or if they are ADA positive at baseline and the titer of one or more post-baseline samples is at least 0.60 titer unit greater than the titer of the baseline sample (treatment-enhanced ADA response). Patients are considered to be ADA negative if they are ADA negative or have missing data at baseline and all post baseline samples are negative, or if they are ADA positive at baseline but do not have any post-baseline samples with a titer that is at least 0.60 titer unit greater than the titer of the baseline sample (treatment unaffected).

The relationship between ADA status and safety, efficacy, PK, and biomarker endpoints is analyzed and reported using standard language and/or terminology.

### H. Biomarker Analyses

During Stage 2, analysis of biomarker testing on tumor tissue and plasma samples is performed.

Non-invasive, simple plasma collection enables the assessment of circulating tumor DNA (ctDNA), which is believed to come from apoptotic remains of tumor cells. The ctDNA is quantified and assessed for its genomic mutations. Beyond associating this information at baseline with clinical efficacy for prognostic and treatment effect evaluation, this information also allows for monitoring of residual disease at the molecular level and for tracking clonal evolution.

Analyses of biomarkers related to tumor biology and the mechanisms of action of polatuzumab vedotin and rituximab are conducted.

Analyses assess prognostic and/or predictive value of candidate biomarkers. The association between candidate biomarkers and OS, PFS and PET-CT CR rate and other measures of efficacy and safety, with treatment and independent of treatment, are explored to assess potential predictive and prognostic value, respectively. The effects of baseline prognostic characteristics, including DLBCL subtypes (i.e., COO) and mutation profiles on efficacy are evaluated using univariate and/or multivariate statistical methods.

The most established DLBCL biomarker subsets to date are molecularly-defined profiles consistent with developmental stage or cell of origin (COO) of the B-cell-originating tumor, i.e., the activated B-cell-like (ABC) profile, the germinal center B-cell-like (GCB) or unclassified profile, the BCL2/MYC double-expressor profile (characterized by elevated expression of both MYC and BCL2), and the BCL2/MYC double-hit profile (translocation in both BCL2 and MYC). In the recent revision of the WHO classification, a new molecular subgroup was defined, the high-grade B-cell lymphoma with MYC and BCL2 and/or BCL6 rearrangements (HGBL DH/TH) (Swerdlow et al., (2016) Blood, 127:2375-2390). More recently, molecular subtypes have been identified that transcend the aforementioned categories, with distinct genotypic, epigenetic and clinical characteristics. Examples include the four genetic subtypes described by Schmitz et al., (2018) N Engl J Med, 378:1396-1407, and the five DLBCL subsets as described by Chapuy et al., (2018) Nat Med, 24:679-690.

Biomarkers for this study include, but are not limited to, any of the biomarkers discussed above, the target of polatuzumab vedotin (CD79b), tumor mutation profiling by next-generation sequencing (NGS), and established prognostic biomarkers in DLBCL (cell-of-origin, BCL2/MYC double-expression and BCL2/MYC double-translocation). A summary of exemplary exploratory biomarkers is provided in Table 3.

TABLE 3
Biomarkers for Retrospective Exploratory Research (Stage 2).

| Sample | Timing (All | |
|---|---|---|
| Type | Mandatory) | Proposed Biomarkers |
| Tumor | Fresh or archival | RNA-based gene expression |
| tissue | | profiling, including but not limited |
| | | to cell-of-origin gene signature |
| | | analysis. |
| | | IHC and proteomic profiling, |
| | | including BCL2 and MYC. |
| | | Translocation profiles, including |
| | | BCL2 and MYC. |
| | | Mutation profiling by NGS, |
| | | including but not limited to CD79b. |
| Plasma | C1D1 (prior to dosing), | ctDNA level and clonal mutation |
| | C2D1 (prior to dosing), | profile. |
| | C5D1 (prior to dosing), | ctDNA as a peripheral measure of |
| | and end of treatment (or | disease biology, prognosis, subsets |
| | discontinuation, | and treatment response. |
| | whichever comes first) | |

ctDNA = circulating tumor DNA; IHC = immunohistochemistry; NGS = next-generation sequencing.

### I. Subgroup Analyses

To assess the consistency of treatment-benefit study results in subgroups defined by demographic and relevant baseline characteristics, OS and PFS in these subgroups are evaluated.

Consistency of treatment benefit is assessed using stratified Cox Proportional hazards models, and hazard ratios with 95% CI are estimated. Forest plots are used to summarize the results.

### J. Interim Safety Analyses

Several interim analyses of safety data are conducted, including examination of FACT/GOG-Ntx12. During the Safety Run-In (Stage 1), safety data is analyzed at least three times. During the RCT stage, safety data is analyzed after the first 10 and 20 patients are randomized in each treatment arm; the frequency of subsequent interim analyses during the RCT stage depends upon the number of ≥Grade 3 peripheral neuropathy events at the second interim analysis. These interim safety analyses assess all recruited patients until they complete study treatment, as deemed necessary.

1. A method for treating diffuse large B-cell lymphoma (DLBCL) in a human in need thereof comprising administering to the human an effective amount of:
(a) an immunoconjugate comprising the formula 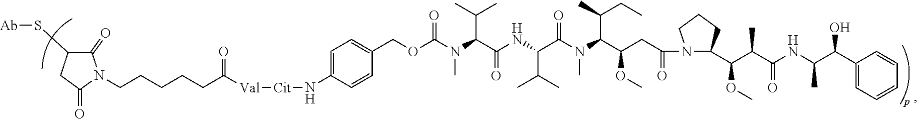
wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) gemcitabine, and
(d) oxaliplatin.

2. The method of claim 1, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.

3. The method of claim 1 or claim 2, wherein the anti-CD79b antibody comprises (i) a heavy chain comprising the amino acid sequence of SEQ ID NO: 36 and (ii) a light chain comprising the amino acid sequence of SEQ ID NO: 35.

4. The method of any one of claims 1-3, wherein p is between 2 and 5.

5. The method of any one of claims 1-4, wherein p is between 3 and 4.

6. The method of any one of claims 1-5, wherein the immunoconjugate is polatuzumab vedotin-piiq.

7. The method of any one of claims 1-2 or 4-5, wherein the anti-CD79b antibody comprises a heavy chain comprising the amino acid sequence of SEQ ID NO: 37 and a light chain comprising the amino acid sequence of SEQ ID NO: 35.

8. The method of any one of claims 1-2 or 4-5, wherein the anti-CD79b antibody comprises a heavy chain comprising the amino acid sequence of SEQ ID NO: 36 and a light chain comprising the amino acid sequence of SEQ ID NO: 38.

9. The method of any one of claims 1-2, 4 or 7, wherein the immunoconjugate is iladatuzumab vedotin.

10. The method of any one of claims 1-9, wherein the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m².

11. The method of anyone of claims 1-10, wherein the immunoconjugate, the rituximab, the gemcitabine, and the oxaliplatin are administered for one or more 21-day cycles.

12. The method of claim 11, wherein the immunoconjugate is administered at a dose of about 1.8 mg/kg each cycle, the rituximab is administered at a dose of about 375 mg/m² each cycle, the gemcitabine is administered at a dose of about 1000 mg/m² each cycle, and the oxaliplatin is administered at a dose of about 100 mg/m² each cycle.

13. The method of claim 11 or claim 12, wherein the immunoconjugate and the rituximab are administered intravenously on Day 1 of each 21-day cycle, and wherein the gemcitabine and the oxaliplatin are administered intravenously on Day 2 of each 21-day cycle.

14. The method of any one of claims 1-13, wherein the rituximab is administered before the immunoconjugate.

15. The method of any one of claims 1-14, wherein the gemcitabine is administered before the oxaliplatin.

16. The method of any one of claims 11-15, wherein the immunoconjugate, the rituximab, the gemcitabine, and the oxaliplatin are administered for up to eight 21-day cycles.

17. The method of any one of claims 11-16, wherein the immunoconjugate, the rituximab, the gemcitabine, and the oxaliplatin are administered for eight 21-day cycles.

18. A method for treating diffuse large B-cell lymphoma (DLBCL) in a human in need thereof comprising administering to the human an effective amount of:
(a) an immunoconjugate comprising the formula 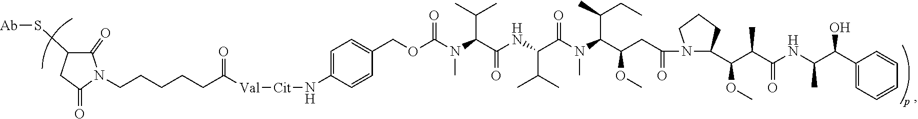
wherein Ab is an anti-CD79b antibody comprising (i) a heavy chain comprising the amino acid sequence of SEQ ID NO: 36 and (ii) a light chain comprising the amino acid sequence of SEQ ID NO: 35,
and wherein p is between 2 and 5,
(b) rituximab,
(c) gemcitabine, and
(d) oxaliplatin;
wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²; and
wherein the immunoconjugate and the rituximab are administered intravenously on Day 1 of each 21-day cycle, and wherein the gemcitabine and the oxaliplatin are administered intravenously on Day 2 of each 21-day cycle.

19. The method of claim 18, wherein the immunoconjugate, the rituximab, the gemcitabine, and the oxaliplatin are administered for at least two, at least three, at least four, at least five, at least six, or at least seven 21-day cycles.

20. The method of claim 18, wherein the immunoconjugate, the rituximab, the gemcitabine, and the oxaliplatin are administered for eight 21-day cycles.

21. The method of any one of claims 18-20, wherein the rituximab is administered before the immunoconjugate.

22. The method of any one of claims 18-21, wherein the gemcitabine is administered before the oxaliplatin.

23. The method of any one of claims 18-22, wherein the immunoconjugate is polatuzumab vedotin-piiq.

24. The method of any one of claims 1-23, wherein the human has received at least one prior therapy for DLBCL.

25. The method of any one of claims 1-24, wherein the human has received at least one prior systemic therapy for DLBCL.

26. The method of any one of claims 1-25, wherein the DLBCL is histologically-confirmed DLBCL, not otherwise specified (NOS), or the human has a history of transformation of indolent disease to DLBCL.

27. The method of any one of claims 1-26, wherein the DLBCL is relapsed or refractory DLBCL.

28. The method of any one of claims 1-27, wherein the human has an Eastern Cooperative Oncology Group (ECOG) performance status of 0, 1, or 2.

29. The method of any one of claims 1-28, wherein the human does not have a planned autologous or allogeneic stem cell transplantation (SCT).

30. The method of any one of claims 1-29, wherein the human is not a candidate for hematopoietic stem cell transplantation (HSCT) prior to administration of the immunoconjugate, the rituximab, the gemcitabine, and the oxaliplatin.

31. The method of any one of claims 1-30, wherein the human is not a candidate for autologous hematopoietic stem cell transplantation (HSCT) prior to administration of the immunoconjugate, the rituximab, the gemcitabine, and the oxaliplatin.

32. The method of any one of claims 1-31, wherein the human has received at least two prior therapies for DLBCL.

33. The method of any one of claims 1-32, wherein the human has not had prior therapy with a combination of gemcitabine and a platinum-based agent.

34. The method of any one of claims 1-33, wherein the human has not received a prior therapy with polatuzumab vedotin-piiq for DLBCL.

35. The method of any one of claims 1-34, wherein the human does not have peripheral neuropathy of greater than Grade 1 according to National Cancer Institute Common Terminology Criteria for Adverse Events, Version 5.0.

36. The method of any one of claims 1-35, wherein the human does not have primary or secondary central nervous system lymphoma.

37. The method of any one of claims 1-36, wherein the human is an adult.

38. The method of claim 37, wherein the human adult has relapsed or refractory diffuse large B-cell lymphoma, not otherwise specified.

39. The method of any one of claims 1-38, wherein administration of the immunoconjugate, the rituximab, the gemcitabine, and the oxaliplatin does not result in peripheral neuropathy of Grade 3 or greater that does not resolve to Grade 1 or lower within 14 days.

40. The method of any one of claims 1-38, wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

41. A method for treating diffuse large B-cell lymphoma (DLBCL) in a human in need thereof comprising administering to the human an effective amount of:
(a) an immunoconjugate comprising the formula 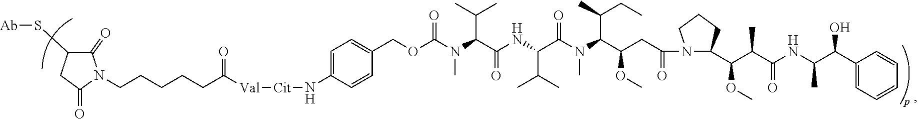
wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) gemcitabine, and
(d) oxaliplatin;
wherein administration of the immunoconjugate, the rituximab, the gemcitabine, and the oxaliplatin does not result in peripheral neuropathy of Grade 3 or greater that does not resolve to Grade 1 or lower within 14 days.

42. A method for treating diffuse large B-cell lymphoma (DLBCL) in a human in need thereof comprising administering to the human an effective amount of:
(a) an immunoconjugate comprising the formula 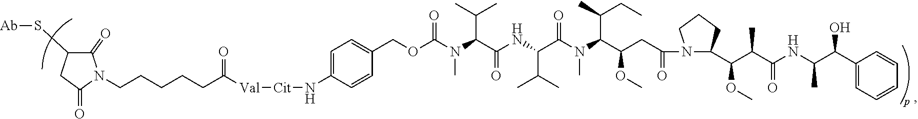
wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) gemcitabine, and
(d) oxaliplatin;
wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

43. A method for treating diffuse large B-cell lymphoma (DLBCL) in a human in need thereof comprising administering to the human an effective amount of:
(a) an immunoconjugate comprising the formula 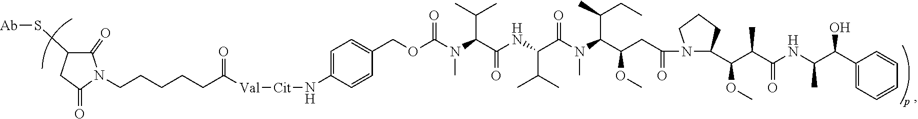
wherein Ab is an anti-CD79b antibody comprising (i) a heavy chain comprising the amino acid sequence of SEQ ID NO: 36 and (ii) a light chain comprising the amino acid sequence of SEQ ID NO: 35,
and wherein p is between 2 and 5,
(b) rituximab,
(c) gemcitabine, and
(d) oxaliplatin;
wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²;
wherein the immunoconjugate and the rituximab are administered intravenously on Day 1 of each 21-day cycle, and wherein the gemcitabine and the oxaliplatin are administered intravenously on Day 2 of each 21-day cycle; and
wherein administration of the immunoconjugate, the rituximab, the gemcitabine, and the oxaliplatin does not result in peripheral neuropathy of Grade 3 or greater that does not resolve to Grade 1 or lower within 14 days.

44. A method for treating diffuse large B-cell lymphoma (DLBCL) in a human in need thereof comprising administering to the human an effective amount of:
(a) an immunoconjugate comprising the formula 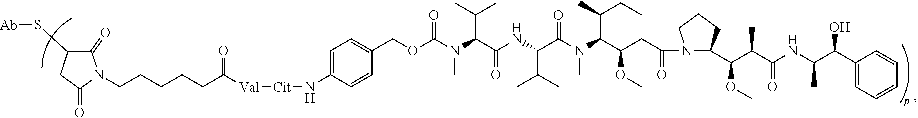
wherein Ab is an anti-CD79b antibody comprising (i) a heavy chain comprising the amino acid sequence of SEQ ID NO: 36 and (ii) a light chain comprising the amino acid sequence of SEQ ID NO: 35,
and wherein p is between 2 and 5,
(b) rituximab,
(c) gemcitabine, and
(d) oxaliplatin;
wherein the immunoconjugate, the rituximab, the gemcitabine, and the oxaliplatin are administered for at least one 21-day cycle, wherein during each cycle the immunoconjugate is administered at a dose of about 1.8 mg/kg, the rituximab is administered at a dose of about 375 mg/m², the gemcitabine is administered at a dose of about 1000 mg/m², and the oxaliplatin is administered at a dose of about 100 mg/m²;
wherein the immunoconjugate and the rituximab are administered intravenously on Day 1 of each 21-day cycle, and wherein the gemcitabine and the oxaliplatin are administered intravenously on Day 2 of each 21-day cycle; and
wherein administration of the immunoconjugate, the rituximab, the gemcitabine, and the oxaliplatin to a plurality of humans results in 33% or fewer of the humans in the plurality experiencing peripheral neuropathy of Grade 3 or greater that does not resolve to Grade 1 or lower within 14 days.

45. The method of any one of claims 41-44, wherein the immunoconjugate, the rituximab, the gemcitabine, and the oxaliplatin are administered for at least two, at least three, at least four, at least five, at least six, or at least seven 21-day cycles.

46. The method of any one of claims 41-44, wherein the immunoconjugate, the rituximab, the gemcitabine, and the oxaliplatin are administered for eight 21-day cycles.

47. The method of any one of claims 41-46, wherein the rituximab is administered before the immunoconjugate.

48. The method of any one of claims 41-47, wherein the gemcitabine is administered before the oxaliplatin.

49. The method of any one of claims 41-48, wherein the immunoconjugate is polatuzumab vedotin-piiq.

50. The method of any one of claims 41-42 or 45-48, wherein the anti-CD79b antibody comprises a heavy chain comprising the amino acid sequence of SEQ ID NO: 37 and a light chain comprising the amino acid sequence of SEQ ID NO: 35.

51. The method of any one of claims 41-42 or 45-48, wherein the anti-CD79b antibody comprises a heavy chain comprising the amino acid sequence of SEQ ID NO: 36 and a light chain comprising the amino acid sequence of SEQ ID NO: 38.

52. The method of any one of claims 41-42, 45-48, or 50, wherein the immunoconjugate is iladatuzumab vedotin.

53. The method of any one of claims 39-52, wherein the immunoconjugate, the rituximab, the gemcitabine, and the oxaliplatin have been administered for at least four 21-day cycles.

54. A kit comprising an immunoconjugate comprising the formula 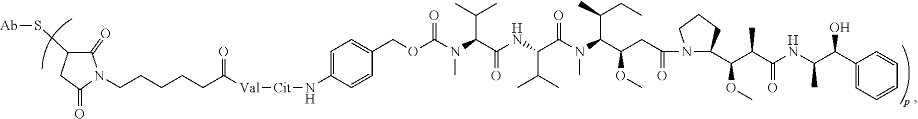
wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with rituximab, gemcitabine, and oxaliplatin for treating a human in need thereof having diffuse large B-cell lymphoma (DLBCL) according to a method of any one of claims 1-53.

55. A kit comprising polatuzumab vedotin-piiq for use in combination with rituximab, gemcitabine, and oxaliplatin for treating a human in need thereof having diffuse large B-cell lymphoma (DLBCL) according to the method of any one of claims 1-53.

56. An immunoconjugate comprising the formula 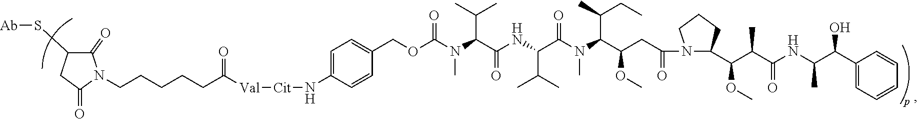
wherein Ab is an anti-CD79b antibody comprising (i) an HVR-H1 comprising the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8
for use in a method of treating diffuse large B-cell lymphoma (DLBCL) in combination with rituximab, gemcitabine, and oxaliplatin according to any one of claims 1-53.

57. A method for treating relapsed or refractory diffuse large B-cell lymphoma (R/R DLBCL) in a human in need thereof, comprising administering to the human polatuzumab vedotin-piiq at a dose of 1.8 mg/kg, rituximab at a dose of 375 mg/m², gemcitabine at a dose of 1000 mg/m², and oxaliplatin at a dose of 100 mg/m².

58. A method for treating relapsed or refractory diffuse large B-cell lymphoma (R/R DLBCL) in a human in need thereof, comprising administering to the human polatuzumab vedotin-piiq at a dose of 1.8 mg/kg, rituximab at a dose of 375 mg/m², gemcitabine at a dose of 1000 mg/m², and oxaliplatin at a dose of 100 mg/m², wherein the polatuzumab vedotin-piiq, the rituximab, the gemcitabine, and the oxaliplatin are administered for at least one 21-day cycle, wherein the polatuzumab vedotin-piiq and the rituximab are administered intravenously on Day 1 of each 21-day cycle, and wherein the gemcitabine and the oxaliplatin are administered intravenously on Day 2 of each 21-day cycle.

59. A method for treating relapsed or refractory diffuse large B-cell lymphoma (R/R DLBCL) in a human in need thereof, comprising administering to the human iladatuzumab vedotin at a dose of 4.8 mg/kg, rituximab at a dose of 375 mg/m², gemcitabine at a dose of 1000 mg/m², and oxaliplatin at a dose of 100 mg/m².

60. A method for treating relapsed or refractory diffuse large B-cell lymphoma (R/R DLBCL) in a human in need thereof, comprising administering to the human iladatuzumab vedotin at a dose of 3.6 mg/kg, rituximab at a dose of 375 mg/m², gemcitabine at a dose of 1000 mg/m², and oxaliplatin at a dose of 100 mg/m².

61. A method for treating relapsed or refractory diffuse large B-cell lymphoma (R/R DLBCL) in a human in need thereof, comprising administering to the human iladatuzumab vedotin at a dose of 4.8 mg/kg, rituximab at a dose of 375 mg/m², gemcitabine at a dose of 1000 mg/m², and oxaliplatin at a dose of 100 mg/m², wherein the iladatuzumab vedotin, the rituximab, the gemcitabine, and the oxaliplatin are administered for at least one 21-day cycle, wherein the iladatuzumab vedotin and the rituximab are administered intravenously on Day 1 of each 21-day cycle, and wherein the gemcitabine and the oxaliplatin are administered intravenously on Day 2 of each 21-day cycle.

62. A method for treating relapsed or refractory diffuse large B-cell lymphoma (R/R DLBCL) in a human in need thereof, comprising administering to the human iladatuzumab vedotin at a dose of 3.6 mg/kg, rituximab at a dose of 375 mg/m², gemcitabine at a dose of 1000 mg/m², and oxaliplatin at a dose of 100 mg/m², wherein the iladatuzumab vedotin, the rituximab, the gemcitabine, and the oxaliplatin are administered for at least one 21-day cycle, wherein the iladatuzumab vedotin and the rituximab are administered intravenously on Day 1 of each 21-day cycle, and wherein the gemcitabine and the oxaliplatin are administered intravenously on Day 2 of each 21-day cycle.
